SEQUENCE LISTING (378 sequences)

SEQ ID NO: 1 - protein, 15 aa, Mus musculus
KNSTLCDLCIGPLKC

SEQ ID NO: 2 - protein, 21 aa, Mus musculus
LMMDYLGNATAVFLLPDDGKM

SEQ ID NO: 3 - protein, 18 aa, Mus musculus
KYTGNASALFILPDQGRM

SEQ ID NO: 4 - protein, 21 aa, Mus musculus
KDYRPSAGNNSLYQDTVVFKC

SEQ ID NO: 5 - protein, 15 aa, Mus musculus
KLTEFTHNSTMDYKC

SEQ ID NO: 6 - protein, 18 aa, Mus musculus
KNLINDYVSNQTQGMIKE

SEQ ID NO: 7 - protein, 18 aa, Mus musculus
KYTGNASALLILPDQGRM

SEQ ID NO: 8 - protein, 18 aa, Mus musculus
KVVLHPNHSVVDIGLIKL

SEQ ID NO: 9 - protein, 15 aa, Mus musculus
KNLFLNHSETASAKD

SEQ ID NO: 10 - protein, 18 aa, Mus musculus
KHSIEHFNNNTDHSHLFT

SEQ ID NO: 11 - protein, 23 aa, Artificial Sequence
  Description of Artificial Sequence Synthetic peptide
RVVGVPYQGNATALFILPSEGKM

SEQ ID NO: 12 - protein, 27 aa, Artificial Sequence
  Description of Artificial Sequence Synthetic peptide
KGVTSVSQIFHSPDLAIRDTFVNASRT

SEQ ID NO: 13 - protein, 23 aa, Artificial Sequence
  Description of Artificial Sequence Synthetic peptide
KHGVIISSTVDTYENGSSVEYRC

SEQ ID NO: 14 - protein, 24 aa, Artificial Sequence
  Description of Artificial Sequence Synthetic peptide
KGPSTPLPEDPNWNVTEFHTTPKM

SEQ ID NO: 15 - protein, 17 aa, Artificial Sequence
  Description of Artificial Sequence Synthetic peptide
RQNQCFYNSSYLNVQRE

SEQ ID NO: 16 - protein, 20 aa, Artificial Sequence
  Description of Artificial Sequence Synthetic peptide
RAVNITSENLIDDVVSLIRG

SEQ ID NO: 17 - protein, 22 aa, Artificial Sequence
  Description of Artificial Sequence Synthetic peptide
KTDNSLLDQALQNDTVFLNMRG

SEQ ID NO: 18 - protein, 21 aa, Artificial Sequence
  Description of Artificial Sequence Synthetic peptide
KSLTFNETYQDISELVYGAKL

SEQ ID NO: 19 - protein, 22 aa, Artificial Sequence
  Description of Artificial Sequence Synthetic peptide
RVYIHPFHLVIHESTCEQLAKA

SEQ ID NO: 20 - protein, 15 aa, Artificial Sequence
  Description of Artificial Sequence Synthetic peptide
KTKPREEQFNSTFRV

SEQ ID NO: 21 - protein, 13 aa, Artificial Sequence
  Description of Artificial Sequence Synthetic peptide
KPREEQFNSTFRV

SEQ ID NO: 22 - protein, 13 aa, Artificial Sequence
  Description of Artificial Sequence Synthetic peptide
KYKNNSDISSTRG

SEQ ID NO: 23 - protein, 11 aa, Artificial Sequence
  Description of Artificial Sequence Synthetic peptide
KTPLTANITKS

SEQ ID NO: 24 - protein, 24 aa, Artificial Sequence
  Description of Artificial Sequence Synthetic peptide
RADGTVNQIEGEATPVNLTEPAKL

SEQ ID NO: 25 - protein, 21 aa, Artificial Sequence
  Description of Artificial Sequence Synthetic peptide
RVLSNNSDANLELINTWVAKN

SEQ ID NO: 26 - protein, 34 aa, Artificial Sequence
  Description of Artificial Sequence Synthetic peptide
RAGENDFSIMYSTRKRSAQLWLGPAAFINHDCKP

SEQ ID NO: 27 - protein, 8 aa, Artificial Sequence
  Description of Artificial Sequence Synthetic peptide
RFSIVMNE

SEQ ID NO: 28 - protein, 13 aa, Artificial Sequence
  Description of Artificial Sequence Synthetic peptide
LFLNHSENATAKD

SEQ ID NO: 29 - protein, 26 aa, Artificial Sequence
  Description of Artificial Sequence Synthetic peptide
KMVSHHNLTTGATLINEQWLLTTAKN

SEQ ID NO: 30 - protein, 20 aa, Artificial Sequence
  Description of Artificial Sequence Synthetic peptide
MNNLSENYENQGRLQVYMNK

SEQ ID NO: 31 - protein, 23 aa, Artificial Sequence
  Description of Artificial Sequence Synthetic peptide
KVGQLQLSHNLSLVILVPQNLKH

SEQ ID NO: 32 - protein, 21 aa, Artificial Sequence
  Description of Artificial Sequence Synthetic peptide
RVLSNNSDANLELINTWVAKN

SEQ ID NO: 33 - protein, 26 aa, Artificial Sequence
  Description of Artificial Sequence Synthetic peptide
KMVSHHNLTIGATLINEQWLLTTAKN

SEQ ID NO: 34 - protein, 27 aa, Artificial Sequence
  Description of Artificial Sequence Synthetic peptide
KNLFLNHSENATAKDIAPTLTLYVGKK

SEQ ID NO: 35 - protein, 13 aa, Artificial Sequence
  Description of Artificial Sequence Synthetic peptide
LFLNHSENATAKD

SEQ ID NO: 36 - protein, 16 aa, Artificial Sequence
  Description of Artificial Sequence Synthetic peptide
KGAFISNFSMTVDGKT

SEQ ID NO: 37 - protein, 24 aa, Artificial Sequence
  Description of Artificial Sequence Synthetic peptide
KAALAAFNAQNNGSNFQLEEISRA

SEQ ID NO: 38 - protein, 26 aa, Artificial Sequence
  Description of Artificial Sequence Synthetic peptide
KILRQQQHLFGSNVTDCSGNFCLFRS

SEQ ID NO: 39 - protein, 23 aa, Artificial Sequence
  Description of Artificial Sequence Synthetic peptide
RQQQHLFGSNVTDCSGNFCLFRS

SEQ ID NO: 40 - protein, 16 aa, Artificial Sequence
  Description of Artificial Sequence Synthetic peptide
GSNVTDCSGNFCLFRS

SEQ ID NO: 41 - protein, 23 aa, Artificial Sequence
  Description of Artificial Sequence Synthetic peptide
RQQQHLFGSNVTDCSGNFCLFRS

SEQ ID NO: 42 - protein, 22 aa, Artificial Sequence
  Description of Artificial Sequence Synthetic peptide
KMLNTSSLLEQLNEQFNWVSRL

SEQ ID NO: 43 - protein, 16 aa, Artificial Sequence
  Description of Artificial Sequence Synthetic peptide
RLANLTQGEDQYYLRV

SEQ ID NO: 44 - protein, 29 aa, Artificial Sequence
  Description of Artificial Sequence Synthetic peptide
KAKENDENCGPTTTVFVGNISEKASDMLI

SEQ ID NO: 45 - protein, 19 aa, Artificial Sequence
  Description of Artificial Sequence Synthetic peptide
RFSDGLESNSSTQFEVKKY

SEQ ID NO: 46 - protein, 33 aa, Artificial Sequence
  Description of Artificial Sequence Synthetic peptide
KADTHDEILEGLNFNLTEIPEAQIHEGFQELLR

SEQ ID NO: 47 - protein, 34 aa, Artificial Sequence
  Description of Artificial Sequence Synthetic peptide
KADTHDEILEGLNFNLTEIPEAQIHEGFQELLRT

SEQ ID NO: 48 - protein, 32 aa, Artificial Sequence
  Description of Artificial Sequence Synthetic peptide
RQLAHQSNSTNIFFSPVSIATAFAMLSLGTKA

SEQ ID NO: 49 - protein, 27 aa, Artificial Sequence
  Description of Artificial Sequence Synthetic peptide
KTIDRLAGKPTHVNVSVVMAEVDGTCY

SEQ ID NO: 50 - protein, 29 aa, Artificial Sequence
  Description of Artificial Sequence Synthetic peptide
RLSLHRPALEDLLLGSEANLTCTLTGLRD

SEQ ID NO: 51 - protein, 45 aa, Artificial Sequence
  Description of Artificial Sequence Synthetic peptide
MPPTPVTPVTPTTPALPAIPISPPPVSAVNKSGPSTVSEPAKSSS

SEQ ID NO: 52 - protein, 21 aa, Artificial Sequence
  Description of Artificial Sequence Synthetic peptide
RVLSNNSDANLELINTWVAKN

SEQ ID NO: 53 - protein, 33 aa, Artificial Sequence
  Description of Artificial Sequence Synthetic peptide
KAHGTSSYTPDKSSFNLATVVAETLGLGVQEES

SEQ ID NO: 54 - protein, 32 aa, Artificial Sequence
  Description of Artificial Sequence Synthetic peptide
RGHLSEEPSENINTPTRLTPQLQMKPMSNRER

SEQ ID NO: 55 - protein, 22 aa, Artificial Sequence
  Description of Artificial Sequence Synthetic peptide
RHTFMGVVSLGSPSGEVSHPRK

SEQ ID NO: 56 - protein, 16 aa, Artificial Sequence
  Description of Artificial Sequence Synthetic peptide
GSNVTDCSGNFCLFRS

SEQ ID NO: 57 - protein, 22 aa, Artificial Sequence
  Description of Artificial Sequence Synthetic peptide
KMLNTSSLLEQLNEQFNWVSRL

SEQ ID NO: 58 - protein, 37 aa, Artificial Sequence
  Description of Artificial Sequence Synthetic peptide
GGGSGGGGAPSGGSGHSGGPSSCGGAPSTSRSRPSRI

SEQ ID NO: 59 - protein, 34 aa, Artificial Sequence; identical to SEQ ID NO: 47
  Description of Artificial Sequence Synthetic peptide

SEQ ID NO: 60 - protein, 33 aa, Artificial Sequence
  Description of Artificial Sequence Synthetic peptide
KYLGNATAIFFLPDEGKLQHLENELTHDIITKF

SEQ ID NO: 61 - protein, 33 aa, Artificial Sequence; identical to SEQ ID NO: 46
  Description of Artificial Sequence Synthetic peptide

SEQ ID NO: 62 - protein, 12 aa, Artificial Sequence
  Description of Artificial Sequence Synthetic peptide
KADLSGITGARN

SEQ ID NO: 63 - protein, 13 aa, Artificial Sequence
  Description of Artificial Sequence Synthetic peptide
KAIGYLNTGYQRQ

SEQ ID NO: 64 - protein, 14 aa, Homo sapiens
KLHVTLYNCSFGRS

SEQ ID NO: 65 - protein, 16 aa, Homo sapiens
RSINVTGQGFSLIQRF

SEQ ID NO: 66 - protein, 25 aa, Homo sapiens
RTEAGAFEYVPDPTFENFTGGVKKQ

SEQ ID NO: 67 - protein, 16 aa, Homo sapiens
RAGPNGTLFVADAYKG

SEQ ID NO: 68 - protein, 29 aa, Homo sapiens
KLLLSSETPIEGKNMSFVNDLTVTQDGRK

SEQ ID NO: 69 - protein, 21 aa, Homo sapiens
RYHYNGSLMDGTLFDSSYSRN

SEQ ID NO: 70 - protein, 21 aa, Homo sapiens
RYHYNGTFLDGTLFDSSHNRM

SEQ ID NO: 71 - protein, 21 aa, Homo sapiens
RYHYNGTLLDGTLFDSSYSRN

SEQ ID NO: 72 - protein, 18 aa, Homo sapiens
RTALFPDLLAQGNASLRL

SEQ ID NO: 73 - protein, 17 aa, Homo sapiens
RLLANSSMLGEGQVLRS

SEQ ID NO: 74 - protein, 24 aa, Homo sapiens
KQVALQTFGNQTTIIPAGGAGYKV

SEQ ID NO: 75 - protein, 23 aa, Homo sapiens
RFTFTSHTPGEHQICLHSNSTKF

SEQ ID NO: 76 - protein, 14 aa, Homo sapiens
KIFIFNQTGIEAKK

SEQ ID NO: 77 - protein, 14 aa, Homo sapiens
KAVLVNNITTGERL

SEQ ID NO: 78 - protein, 19 aa, Homo sapiens
RLQQDVLQFQKNQTNLERK

SEQ ID NO: 79 - protein, 23 aa, Homo sapiens
KVVMDIPYELWNETSAEVADLKK

SEQ ID NO: 80 - protein, 18 aa, Homo sapiens
KLNITNIWVLDYFGGPKI

SEQ ID NO: 81 - protein, 23 aa, Homo sapiens
KYHYNASLLDGTLLDSTWNLGKT

SEQ ID NO: 82 - protein, 34 aa, Homo sapiens
RKTTWLQAASLLFVDNPVGTGFSYVNGSGAYAKD

SEQ ID NO: 83 - protein, 32 aa, Homo sapiens
RCFATTYYLSEGGGLIFRNVTGEPNCRPPTRG

SEQ ID NO: 84 - protein, 21 aa, Homo sapiens
RYHYNGTLLDGTSFDTSYSKG

SEQ ID NO: 85 - protein, 22 aa, Homo sapiens
RGILNATISVAEINHPVTTYKT

SEQ ID NO: 86 - protein, 24 aa, Homo sapiens
RGLVMNYPHITNLTNLGQSTEYRH

SEQ ID NO: 87 - protein, 22 aa, Homo sapiens
RLLNTTDVYLLPSLNPDGFERA

SEQ ID NO: 88 - protein, 15 aa, Homo sapiens
RALGFENATQALGRA

SEQ ID NO: 89 - protein, 22 aa, Homo sapiens
RVFPYISVMVNNGSLSYDHSKD

SEQ ID NO: 90 - protein, 33 aa, Homo sapiens
RYHYNCSLLDGTQLFTSHDYGAPQEATLGANKV

SEQ ID NO: 91 - protein, 19 aa, Homo sapiens
RYSLNVTYNYPVHYFDGRK

SEQ ID NO: 92 - protein, 22 aa, Homo sapiens
RDIENLKDASSFLAEWQNITKG

SEQ ID NO: 93 - protein, 24 aa, Homo sapiens
RLLIAGTNSSDLQQILSLLESNKD

SEQ ID NO: 94 - protein, 15 aa, Homo sapiens
KSLVTQYLNATGNRW

SEQ ID NO: 95 - protein, 24 aa, Homo sapiens
KILAPAYFILGGNQSGEGCVITRD

SEQ ID NO: 96 - protein, 15 aa, Homo sapiens
RTVLENSTSYEEAKN

SEQ ID NO: 97 - protein, 22 aa, Homo sapiens
RFKLEWLGNCSGLNDETYGYKE

SEQ ID NO: 98 - protein, 23 aa, Homo sapiens
RVLGFKPKPPKNESLETYPVMKY

SEQ ID NO: 99 - protein, 22 aa, Homo sapiens
KYLQPLLAVQFTNLTMDTEIRI

SEQ ID NO: 100 - protein, 25 aa, Homo sapiens
KLHVGYLQPLVAVQVSFAPNNTGKE

SEQ ID NO: 101 - protein, 32 aa, Homo sapiens
KLHVGYLQPLVAVQVSFAPNNTGKEVTVECKI

SEQ ID NO: 102 - protein, 20 aa, Homo sapiens
KILLTCSLNDSATEVTGHRW

SEQ ID NO: 103 - protein, 18 aa, Homo sapiens
KITDSEDKALMNGSESRF

SEQ ID NO: 104 - protein, 22 aa, Homo sapiens
RLLDAENKVVANGTGTQGQLKV

SEQ ID NO: 105 - protein, 15 aa, Homo sapiens
KVVANGTGTQGQLKV

SEQ ID NO: 106 - protein, 23 aa, Homo sapiens
KIIISPEENVTLTCTAENQLERT

SEQ ID NO: 107 - protein, 21 aa, Homo sapiens
KLGDCISEDSYPDGNITWYRN

SEQ ID NO: 108 - protein, 17 aa, Homo sapiens
RLNLSENYTLSISNARI

SEQ ID NO: 109 - protein, 28 aa, Homo sapiens
RTVNSLNVSAISIPEHDEADEISDENRE

SEQ ID NO: 110 - protein, 30 aa, Homo sapiens
RTVNSLNVSAISIPEHDEADEISDENREKV

SEQ ID NO: 111 - protein, 12 aa, Homo sapiens
KGSLSYLNVTRK

SEQ ID NO: 112 - protein, 15 aa, Homo sapiens
KYYKGSLSYLNVTRK

SEQ ID NO: 113 - protein, 16 aa, Homo sapiens
KYSVANDTGFVDIPKQ

SEQ ID NO: 114 - protein, 20 aa, Homo sapiens
RQQMENYPKNNHTASILDRM

SEQ ID NO: 115 - protein, 22 aa, Homo sapiens
KMLNTSSLLEQLNEQFNWVSRL

SEQ ID NO: 116 - protein, 30 aa, Homo sapiens
KGHHVSQEPWNSSITLTSQAGAVFQSFAKF

SEQ ID NO: 117 - protein, 31 aa, Homo sapiens
KDTGELNVTSILDREETPFFLLTGYALDARG

SEQ ID NO: 118 - protein, 25 aa, Homo sapiens
KHNNDTQHIWESDSNEFSVIADPRG

SEQ ID NO: 119 - protein, 22 aa, Homo sapiens
KYLNFVKGVVDSDDLPLNVSRE

SEQ ID NO: 120 - protein, 24 aa, Homo sapiens
KFLYNFTQIPHLAGTEQNFQLAKQ

SEQ ID NO: 121 - protein, 20 aa, Homo sapiens
RGVAYINADSSIEGNYTLRV

SEQ ID NO: 122 - protein, 24 aa, Homo sapiens
KTYSVSFDSLFSAVKNFTEIASKF

SEQ ID NO: 123 - protein, 18 aa, Homo sapiens
RVDCTPLMYSLVHNLTKE

SEQ ID NO: 124 - protein, 19 aa, Homo sapiens
KVPYNVGPGFTGNFSTQKV

SEQ ID NO: 125 - protein, 18 aa, Homo sapiens
KTPMTNSSIQFLDNAFRK

SEQ ID NO: 126 - protein, 13 aa, Homo sapiens
KYYNYTLSINGKA

SEQ ID NO: 127 - protein, 17 aa, Homo sapiens
RDLGPTLANSTHHNVRL

SEQ ID NO: 128 - protein, 28 aa, Homo sapiens
RMELSMGPIQANHTGTGLLLTLQPEQKF

SEQ ID NO: 129 - protein, 29 aa, Homo sapiens
RRMELSMGPIQANHTGTGLLLTLQPEQKF

SEQ ID NO: 130 - protein, 26 aa, Homo sapiens
RTYTYADTPDDFQLHNFSLPEEDTKL

SEQ ID NO: 131 - protein, 24 aa, Homo sapiens
RDIVGNLTELESEDIQIEALLMRA

SEQ ID NO: 132 - protein, 25 aa, Homo sapiens
KLDSFGPINPTLNTTYSFLTTFFKE

SEQ ID NO: 133 - protein, 24 aa, Homo sapiens
RTAADTTGLQPILNQFTPANISRQ

SEQ ID NO: 134 - protein, 31 aa, Homo sapiens
RLTGSDVNLLAENLLSPEDMVLFHNLTQPRG

SEQ ID NO: 135 - protein, 11 aa, Homo sapiens
KLLNNLTSIKI

SEQ ID NO: 136 - protein, 12 aa, Homo sapiens
RGHTLTLNFTRN

SEQ ID NO: 137 - protein, 22 aa, Homo sapiens
KSGPKNMTFDLPSDATVVLNRS

SEQ ID NO: 138 - protein, 19 aa, Homo sapiens
KIAVQFGPGFSWIANFTKA

SEQ ID NO: 139 - protein, 10 aa, Homo sapiens
KWQMNFTVRY

SEQ ID NO: 140 - protein, 17 aa, Homo sapiens
RTQNFTLLVQGSPELKT

SEQ ID NO: 141 - protein, 17 aa, Homo sapiens
RGVFITNETGQPLIGKV

SEQ ID NO: 142 - protein, 20 aa, Homo sapiens
KCNMINGTDGDSFHPLITKD

SEQ ID NO: 143 - protein, 19 aa, Homo sapiens
RTMVFPVMYLNESVHIDKE

SEQ ID NO: 144 - protein, 24 aa, Homo sapiens
RTMVFPVMYLNESVHIDKETASRL

SEQ ID NO: 145 - protein, 26 aa, Homo sapiens
RLEHQFAVGEDSGRNLSAPVTLNLRD

SEQ ID NO: 146 - protein, 32 aa, Homo sapiens
RATLITFLCDRDAGVGFPEYQEEDNSTYNFRW

SEQ ID NO: 147 - protein, 29 aa, Homo sapiens
RHGNLYDLKPLGLNDTIVSAGEYTYYFRV

SEQ ID NO: 148 - protein, 23 aa, Homo sapiens
KIKTNITLVCKPGDLESAPVLRT

SEQ ID NO: 149 - protein, 21 aa, Homo sapiens
RSLLEFNTTVSCDQQGTNHRV

SEQ ID NO: 150 - protein, 21 aa, Homo sapiens
KTNITLVCKPGDLESAPVLRT

SEQ ID NO: 151 - protein, 14 aa, Homo sapiens
KLVLPAKNTTNLKT

SEQ ID NO: 152 - protein, 24 aa, Homo sapiens
RSHGVQTMVVLNNLEPNTTYEIRV

SEQ ID NO: 153 - protein, 13 aa, Homo sapiens
RSCINESAIDSRG

SEQ ID NO: 154 - protein, 20 aa, Homo sapiens
KVMELEKEIANATAKPEDRN

SEQ ID NO: 155 - protein, 22 aa, Homo sapiens
RTSLELWMHTDPVSQKNESVRN

SEQ ID NO: 156 - protein, 17 aa, Homo sapiens
RAEPPLNASASDQGEKV

SEQ ID NO: 157 - protein, 39 aa, Homo sapiens
KLGNTISSLFGGGTTPDAKENGTDTVQEEEESPAEGSKD

SEQ ID NO: 158 - protein, 18 aa, Homo sapiens
RLSALDNLLNHSSMFLKG

SEQ ID NO: 159 - protein, 29 aa, Homo sapiens
RQTVHFQISSQLQFSPEEVLGMVLNYSRS

SEQ ID NO: 160 - protein, 13 aa, Homo sapiens
RVFGSQNLTTVKL

SEQ ID NO: 161 - protein, 11 aa, Homo sapiens
KVINETWAWKN

SEQ ID NO: 162 - protein, 20 aa, Homo sapiens
KVINETWAWKNATLAEQAKL

SEQ ID NO: 163 - protein, 25 aa, Homo sapiens
RLPQVSPADSGEYVCRVENGSGPKE

SEQ ID NO: 164 - protein, 21 aa, Homo sapiens
KFLNDSIVDPVDSEWFGFYRS

SEQ ID NO: 165 - protein, 23 aa, Homo sapiens
RMHIFQNGSLVIHDVAPEDSGRY

SEQ ID NO: 166 - protein, 27 aa, Homo sapiens
KDVVTAAGDMLKDNATEEEILVYLEKT

SEQ ID NO: 167 - protein, 19 aa, Homo sapiens
RNLEKNSTKQEILAALEKG

SEQ ID NO: 168 - protein, 23 aa, Homo sapiens
KGQTALGFLYASGLGVNSSQAKA

SEQ ID NO: 169 - protein, 26 aa, Homo sapiens
RDAGVLNDTLVIFTSDNGIPFPSGRT

SEQ ID NO: 170 - protein, 34 aa, Homo sapiens
RNALLLLADDGGFESGAYNNSAIATPHLDALARR

SEQ ID NO: 171 - protein, 27 aa, Homo sapiens
RYPQDYQFYIQNFTALPLNTVVPPQRQ

SEQ ID NO: 172 - protein, 40 aa, Homo sapiens
KAGYFNFTSATITYLAQEDGPVVIGSTSAPGQGGILAQRE

SEQ ID NO: 173 - protein, 19 aa, Homo sapiens
RIAPASNVSHTVVLRPLKA

SEQ ID NO: 174 - protein, 16 aa, Homo sapiens
RLAVTNTTMTGTVLKM

SEQ ID NO: 175 - protein, 19 aa, Homo sapiens
KFNLTETSEAEIHQSFQHL

SEQ ID NO: 176 - protein, 17 aa, Homo sapiens
FLSLGAHNTTLTEILKG

SEQ ID NO: 177 - protein, 25 aa, Homo sapiens
LSISTALAFLSLGAHNTTLTEILKG

SEQ ID NO: 178 - protein, 15 aa, Homo sapiens
RQLAHQSNSTNIFFS

SEQ ID NO: 179 - protein, 24 aa, Homo sapiens
KAALAAFNAQNNGSNFQLEEISRA

SEQ ID NO: 180 - protein, 17 aa, Homo sapiens
FNLTETSEAEIHQSFQH

SEQ ID NO: 181 - protein, 23 aa, Homo sapiens
RGFGVAIVGNYTAALPTEAALRT

SEQ ID NO: 182 - protein, 18 aa, Homo sapiens
YVLDYLNETQQLTPEIKS

SEQ ID NO: 183 - protein, 19 aa, Homo sapiens
NLVPVPITNATLDQITGKW

SEQ ID NO: 184 - protein, 18 aa, Homo sapiens
KYLGNATAIFFLPDEGKL

SEQ ID NO: 185 - protein, 15 aa, Homo sapiens
RQLAHQSNSTNIFFS

SEQ ID NO: 186 - protein, 18 aa, Homo sapiens
KYTGNASALFILPDQDKM

SEQ ID NO: 187 - protein, 15 aa, Homo sapiens
NHMGNVTFTIPANRE

SEQ ID NO: 188 - protein, 18 aa, Homo sapiens
RFSDGLESNSSTQFEVKK

SEQ ID NO: 189 - protein, 18 aa, Homo sapiens
KVVLHPNYSQVDIGLIKL

SEQ ID NO: 190 - protein, 15 aa, Homo sapiens
KNLFLNHSENATAKD

SEQ ID NO: 191 - protein, 21 aa, Homo sapiens
RVLSNNSDANLELINTWVAKN

SEQ ID NO: 192 - protein, 14 aa, Homo sapiens
HFFAPQNLTNMNKN

SEQ ID NO: 193 - protein, 16 aa, Homo sapiens
KGAFISNFSMTVDGKT

SEQ ID NO: 194 - protein, 14 aa, Homo sapiens
NQLVDALTTWQNKT

SEQ ID NO: 195 - protein, 14 aa, Homo sapiens
KFLNDTMAVYEAKL

SEQ ID NO: 196 - protein, 15 aa, Homo sapiens
RIYSGILNLSDITKD

SEQ ID NO: 197 - protein, 14 aa, Homo sapiens
KLNAENNATFYFKI

SEQ ID NO: 198 - protein, 25 aa, Artificial Sequence
  Description of Artificial Sequence Synthetic peptide
GVTSVSQIFHSPDLAIRDTFVDASR

SEQ ID NO: 199 - protein, 21 aa, Artificial Sequence
  Description of Artificial Sequence Synthetic peptide
VYIHPFHLVIHDESTCEQLAK

SEQ ID NO: 200 - protein, 22 aa, Artificial Sequence
  Description of Artificial Sequence Synthetic peptide
VTQDLTLIEESLTSEFIHDIDR

SEQ ID NO: 201 - protein, 25 aa, Artificial Sequence
  Description of Artificial Sequence Synthetic peptide
SRVYLQGLIDYYLFGDSSTVLEDSK

SEQ ID NO: 202 - protein, 18 aa, Artificial Sequence
  Description of Artificial Sequence Synthetic peptide
AVDITSENLIDDVVSLIR

SEQ ID NO: 203 - protein, 18 aa, Artificial Sequence
  Description of Artificial Sequence Synthetic peptide
YQFNTNVVFSNDGTLVDR

SEQ ID NO: 204 - protein, 21 aa, Artificial Sequence
  Description of Artificial Sequence Synthetic peptide
HGVIISSTVDTYEDGSSVEYR

SEQ ID NO: 205 - protein, 15 aa, Artificial Sequence
  Description of Artificial Sequence Synthetic peptide
QNQCFYDSSYLNVQR

SEQ ID NO: 206 - protein, 21 aa, Artificial Sequence
  Description of Artificial Sequence Synthetic peptide
VVGVPYQGDATALFILPSEGK

SEQ ID NO: 207 - protein, 22 aa, Artificial Sequence
  Description of Artificial Sequence Synthetic peptide
GPSTPLPEDPNWDVTEFHTTPK

SEQ ID NO: 208 - protein, 19 aa, Artificial Sequence
  Description of Artificial Sequence Synthetic peptide
SLTFDETYQDISELVYGAK

SEQ ID NO: 209 - protein, 20 aa, Artificial Sequence
  Description of Artificial Sequence Synthetic peptide
TDNSLLDQALQDDTVFLNMR

SEQ ID NO: 210 - protein, 13 aa, Homo sapiens
IYSGILNLSDITK

SEQ ID NO: 211 - protein, 21 aa, Homo sapiens
KFLSSSPHLPPSSYFNASGRA

SEQ ID NO: 212 - protein, 19 aa, Gallus sp.
KWTNDLGSNMTIGAVNSRG

SEQ ID NO: 213 - protein, 19 aa, Saccharomyces sp.
SMTNETSDRPLVHFTPNKG

SEQ ID NO: 214 - protein, 14 aa, Saccharomyces sp.
KNPVLAANSTQFRD

SEQ ID NO: 215 - protein, 20 aa, Saccharomyces sp.
YFVGSFNGTHFEAFDNQSRV

SEQ ID NO: 216 - protein, 17 aa, Saccharomyces sp.
KAEPILNISNAGPWSRF

SEQ ID NO: 217 - protein, 11 aa, Saccharomyces sp.
RFATNTTLTKA

SEQ ID NO: 218 - protein, 14 aa, Saccharomyces sp.
LVYAVNTTQTISKS

SEQ ID NO: 219 - protein, 30 aa, Saccharomyces sp.
FMTTGNALGSVNMTTGVDNLFYIDKFQVRE

SEQ ID NO: 220 - protein, 32 aa, Homo sapiens; identical to SEQ ID NO: 48

SEQ ID NO: 221 - protein, 34 aa, Homo sapiens; identical to SEQ ID NO: 47

SEQ ID NO: 222 - protein, 18 aa, Homo sapiens
KYLGNATAIFFLPDEGKL

SEQ ID NO: 223 - protein, 13 aa, Gallus sp.
GWVIPMGLIHNRT

SEQ ID NO: 224 - protein, 9 aa, Bos sp.
SRNLTKDRC

SEQ ID NO: 225 - protein, 34 aa, Gallus sp.
KYNLTSVLMAMGITDVFSSSANLSGISSAESLKI

SEQ ID NO: 226 - protein, 15 aa, Bos sp.
LCPDCPLLAPLDDSR

SEQ ID NO: 227 - protein, 9 aa, Homo sapiens
AKPALEDLR

SEQ ID NO: 228 - protein, 11 aa, Homo sapiens
  MOD_RES (7): De-amidated Asn
LEALKENGGAR

SEQ ID NO: 229 - protein, 11 aa, Homo sapiens
QGLLPVLESFK

SEQ ID NO: 230 - protein, 11 aa, Homo sapiens
DLATVYVDVLK

SEQ ID NO: 231 - protein, 10 aa, Homo sapiens
VQPYLDDFQK

SEQ ID NO: 232 - protein, 11 aa, Homo sapiens
THLAPYSDELR

SEQ ID NO: 233 - protein, 12 aa, Homo sapiens
VSFLSALEEYTK

SEQ ID NO: 234 - protein, 13 aa, Homo sapiens
DYVSQFEGSALGK

SEQ ID NO: 235 - protein, 13 aa, Homo sapiens
VKDLATVYVDVLK

SEQ ID NO: 236 - protein, 13 aa, Homo sapiens
VEPLRAELQEGAR

SEQ ID NO: 237 - protein, 13 aa, Homo sapiens
THLAPYSDELRQR

SEQ ID NO: 238 - protein, 14 aa, Homo sapiens
LLDNWDSVTSTFSK

SEQ ID NO: 239 - protein, 15 aa, Homo sapiens
DLATVYVDVLKDSGR

SEQ ID NO: 240 - protein, 17 aa, Homo sapiens
DSGRDYVSQFEGSALGK

SEQ ID NO: 241 - protein, 16 aa, Homo sapiens
LLDNWDSVTSTFSKLR

SEQ ID NO: 242 - protein, 16 aa, Homo sapiens
EQLGPVTQEFWDNLEK

SEQ ID NO: 243 - protein, 18 aa, Homo sapiens
DYVSQFEGSALGKQLNLK

SEQ ID NO: 244 - protein, 18 aa, Homo sapiens
AHVDALRTHLAPYSDELR

SEQ ID NO: 245 - protein, 18 aa, Homo sapiens
LREQLGPVTQEFWDNLEK

SEQ ID NO: 246 - protein, 9 aa, Homo sapiens
GYTQQLAFR

SEQ ID NO: 247 - protein, 8 aa, Homo sapiens
FYYIYNEK

SEQ ID NO: 248 - protein, 10 aa, Homo sapiens
DFDFVPPVVR

SEQ ID NO: 249 - protein, 9 aa, Homo sapiens
  MOD_RES (7): Oxidized Met
YYTYLIMNK

SEQ ID NO: 250 - protein, 12 aa, Homo sapiens
TIYTPGSTVLYR

SEQ ID NO: 251 - protein, 14 aa, Homo sapiens
IPIEDGSGEVVLSR

SEQ ID NO: 252 - protein, 13 aa, Homo sapiens
IFTVNHKLLPVGR

SEQ ID NO: 253 - protein, 14 aa, Homo sapiens
LVAYYTLIGASGQR

SEQ ID NO: 254 - protein, 14 aa, Homo sapiens
  MOD_RES (10): Oxidized Met
AGDFLEANYMNLQR

SEQ ID NO: 255 - protein, 16 aa, Homo sapiens
SNLDEDIIAEENIVSR

SEQ ID NO: 256 - protein, 17 aa, Homo sapiens
VEGTAFVIFGIQDGEQR

SEQ ID NO: 257 - protein, 16 aa, Homo sapiens
TELRPGETLNVNFLLR

SEQ ID NO: 258 - protein, 17 aa, Homo sapiens
LSINTHPSQKPLSITVR

SEQ ID NO: 259 - protein, 18 aa, Homo sapiens
AYYENSPQQVFSTEFEVK

SEQ ID NO: 260 - protein, 20 aa, Homo sapiens
  MOD_RES (11): Oxidized Met
ILLQGTPVAQMTEDAVDAER

SEQ ID NO: 261 - protein, 21 aa, Homo sapiens
  MOD_RES (10): Oxidized Met
  One of the Asn residues in this sequence is de-amidated
TVLTPATNHMGNVTFTIPANR

SEQ ID NO: 262 - protein, 21 aa, Homo sapiens
  MOD_RES (10): Oxidized Met
  Two of the Asn residues in this sequence are de-amidated
TVLTPATNHMGNVTFTIPANR

SEQ ID NO: 263 - protein, 22 aa, Homo sapiens
EPGQDLVVLPLSITTDFIPSFR

SEQ ID NO: 264 - protein, 7 aa, Homo sapiens
EFHLHLR

SEQ ID NO: 265 - protein, 13 aa, Homo sapiens
GSFEFPVGDAVSK

SEQ ID NO: 266 - protein, 10 aa, Homo sapiens
SFFPENWLWR

SEQ ID NO: 267 - protein, 14 aa, Homo sapiens
TTNIQGINLLFSSR

SEQ ID NO: 268 - protein, 14 aa, Homo sapiens
YIYGKPVQGVAYVR

SEQ ID NO: 269 - protein, 15 aa, Homo sapiens
HLVPGAPFLLQALVR

SEQ ID NO: 270 - protein, 16 aa, Homo sapiens
  MOD_RES (8): De-amidated Asn
FSDGLESNSSTQFEVK

SEQ ID NO: 271 - protein, 17 aa, Homo sapiens
GHLFLQTDQPIYNPGQR

SEQ ID NO: 272 - protein, 18 aa, Homo sapiens
RGHLFLQTDQPIYNPGQR

SEQ ID NO: 273 - protein, 19 aa, Homo sapiens
ALEILQEEDLIDEDDIPVR

SEQ ID NO: 274 - protein, 26 aa, Homo sapiens
VTASDPLDTLGSEGALSPGGVASLLR

SEQ ID NO: 275 - protein, 10 aa, Homo sapiens
QGIPFFGQVR

SEQ ID NO: 276 - protein, 10 aa, Homo sapiens
  MOD_RES (8): Oxidized Met
LSFYYLIMAK

SEQ ID NO: 277 - protein, 13 aa, Homo sapiens
SKAIGYLNTGYQR

SEQ ID NO: 278 - protein, 13 aa, Homo sapiens
NQGNTWLTAFVLK

SEQ ID NO: 279 - protein, 13 aa, Homo sapiens
  One of the Asn residues in this sequence is de-amidated
NQGNTWLTAFVLK

SEQ ID NO: 280 - protein, 15 aa, Homo sapiens
ALLAYAFALAGNQDK

SEQ ID NO: 281 - protein, 14 aa, Homo sapiens
TEVSSNHVLIYLDK

SEQ ID NO: 282 - protein, 14 aa, Homo sapiens
TEHPFTVEEFVLPK

SEQ ID NO: 283 - protein, 16 aa, Homo sapiens
ALLAYAFALAGNQDKR

SEQ ID NO: 284 - protein, 18 aa, Homo sapiens
LLIYAVLPTGDVIGDSAK

SEQ ID NO: 285 - protein, 18 aa, Homo sapiens
  MOD_RES (17): Oxidized Met
LLLQQVSLPELPGEYSMK

SEQ ID NO: 286 - protein, 19 aa, Homo sapiens
LHTEAQIQEEGTVVELTGR

SEQ ID NO: 287 - protein, 19 aa, Homo sapiens
  MOD_RES (3): De-amidated Asn
VSNQTLSLFFTVLQDVPVR

SEQ ID NO: 288 - protein, 9 aa, Homo sapiens
HINIDQFVR

SEQ ID NO: 289 - protein, 10 aa, Homo sapiens
SPAFTDLHLR

SEQ ID NO: 290 - protein, 11 aa, Homo sapiens
TEVIPPLIENR

SEQ ID NO: 291 - protein, 11 aa, Homo sapiens
  Two of the Asn residues in this sequence are de-amidated
NRNNALDFVTK

SEQ ID NO: 292 - protein, 11 aa, Homo sapiens
  MOD_RES (1): De-amidated Asn
  MOD_RES (3)..(4): De-amidated Asn
NRNNALDFVTK

SEQ ID NO: 293 - protein, 15 aa, Homo sapiens
TSSFALNLPTLPEVK

SEQ ID NO: 294 - protein, 15 aa, Homo sapiens
  One of the Asn residues in this sequence is de-amidated
FNSSYLQGTNQITGR

SEQ ID NO: 295 - protein, 16 aa, Homo sapiens
ATFQTPDFIVPLTDLR

SEQ ID NO: 296 - protein, 18 aa, Homo sapiens
FSVPAGIVIPSFQALTAR

SEQ ID NO: 297 - protein, 17 aa, Homo sapiens
  MOD_RES (9): De-amidated Asn
FEVDSPVYNATWSASLK

SEQ ID NO: 298 - protein, 21 aa, Homo sapiens
IGVELTGRTLADLTLLDSPIK

SEQ ID NO: 299 - protein, 15 aa, Homo sapiens
  MOD_RES (12): De-amidated Asn
VCQDCPLLAPLNDTR

SEQ ID NO: 300 - protein, 16 aa, Homo sapiens
KVCQDCPLLAPLNDTR

SEQ ID NO: 301 - protein, 16 aa, Homo sapiens
  MOD_RES (13): De-amidated Asn
KVCQDCPLLAPLNDTR

SEQ ID NO: 302 - protein, 22 aa, Homo sapiens
  One of the Asn residues in this sequence is de-amidated
AALAAFNAQNNGSNFQLEEISR

SEQ ID NO: 303 - protein, 22 aa, Homo sapiens
  Two of the Asn residues in this sequence are de-amidated
AALAAFNAQNNGSNFQLEEISR

SEQ ID NO: 304 - protein, 9 aa, Homo sapiens
QVVAGLNFR

SEQ ID NO: 305 - protein, 11 aa, Homo sapiens
TVGSDTFYSFK

SEQ ID NO: 306 - protein, 12 aa, Homo sapiens
  One of the Asn residues in this sequence is de-amidated
LNAENNATFYFK

SEQ ID NO: 307 - protein, 15 aa, Homo sapiens
  One of the Asn residues in this sequence is de-amidated
YNSQNQSNNQFVLYR

SEQ ID NO: 308 - protein, 15 aa, Homo sapiens
  Three of the Asn residues in this sequence are de-amidated
YNSQNQSNNQFVLYR

SEQ ID NO: 309 - protein, 8 aa, Homo sapiens
RPHFFFPK

SEQ ID NO: 310 - protein, 12 aa, Homo sapiens
ASSIIDELFQDR

SEQ ID NO: 311 - protein, 14 aa, Homo sapiens
  MOD_RES (3): De-amidated Asn
LANLTQGEDQYYLR

SEQ ID NO: 312 - protein, 17 aa, Homo sapiens
LFDSDPITVTVPVEVSR

SEQ ID NO: 313 - protein, 9 aa, Homo sapiens
  MOD_RES (6): De-amidated Asn
QKYTVNQCR

SEQ ID NO: 314 - protein, 12 aa, Homo sapiens
ALYLQYTDETFR

SEQ ID NO: 315 - protein, 20 aa, Homo sapiens
  MOD_RES (2): De-amidated Asn
ENLTAPGSDSAVFFEQGTTR

SEQ ID NO: 316 - protein, 7 aa, Homo sapiens
LWWLDLK

SEQ ID NO: 317 - protein, 8 aa, Homo sapiens
RLWWLDLK

SEQ ID NO: 318 - protein, 11 aa, Homo sapiens
NFPSPVDAAFR

SEQ ID NO: 319 - protein, 11 aa, Homo sapiens
YYCFQGNQFLR

SEQ ID NO: 320 - protein, 24 aa, Homo sapiens
EVGTPHGIILDSVDAAFICPGSSR

SEQ ID NO: 321 - protein, 14 aa, Homo sapiens
  MOD_RES (13): De-amidated Asn
AGLQAFFQVQECNK

SEQ ID NO: 322 - protein, 16 aa, Homo sapiens
  MOD_RES (10): De-amidated Asn
EHEGAIYPDNTTDFQR

SEQ ID NO: 323 - protein, 20 aa, Homo sapiens
  MOD_RES (2): De-amidated Asn
ENLTAPGSDSAVFFEQGTTR

SEQ ID NO: 324 - protein, 25 aa, Homo sapiens
  One of the Asn residues in this sequence is de-amidated
ELHHLQEQNVSNAFLDKGEFYIGSK

SEQ ID NO: 325 - protein, 12 aa, Homo sapiens
HYDGSYSTFGER

SEQ ID NO: 326 - protein, 19 aa, Homo sapiens
VSNQTLSLFFTVLQDVPVR

SEQ ID NO: 327 - protein, 19 aa, Homo sapiens
  MOD_RES (3): De-amidated Asn
VSNQTLSLFFTVLQDVPVR

SEQ ID NO: 328 - protein, 22 aa, Homo sapiens
  MOD_RES (9): De-amidated Asn
GCVLLSYLNETVTVSASLESVR

SEQ ID NO: 329 - protein, 27 aa, Homo sapiens
  MOD_RES (3): De-amidated Asn
VSNQTLSLFFTVLQDVPVRDLKPAIVK

SEQ ID NO: 330 - protein, 35 aa, Homo sapiens
  MOD_RES (28): Oxidized Met
  Two of the Asn residues in this sequence are de-amidated
GNEANYYSNATTDEHGLVQFSINTTNVMGTSLTVR

SEQ ID NO: 331 - protein, 12 aa, Homo sapiens
  MOD_RES (9): De-amidated Asn
ITYSIVQTNCSK

SEQ ID NO: 332 - protein, 12 aa, Homo sapiens
  One of the Asn residues in this sequence is de-amidated
LNAENNATFYFK

SEQ ID NO: 333 - protein, 12 aa, Homo sapiens
  Two of the Asn residues in this sequence are de-amidated
LNAENNATFYFK

SEQ ID NO: 334 - protein, 15 aa, Homo sapiens
  One of the Asn residues in this sequence is de-amidated
YNSQNQSNNQFVLYR

SEQ ID NO: 335 - protein, 15 aa, Homo sapiens
  Two of the Asn residues in this sequence are de-amidated
YNSQNQSNNQFVLYR

SEQ ID NO: 336 - protein, 16 aa, Homo sapiens
  One of the Asn residues in this sequence is de-amidated
KYNSQNQSNNQFVLYR

SEQ ID NO: 337 - protein, 23 aa, Homo sapiens
HGIQYFNNNTQHSSLFMLNEVKR

SEQ ID NO: 338 - protein, 23 aa, Homo sapiens
HGIQYFNNNTQHSSLFMLNEVKR

SEQ ID NO: 339 - protein, 23 aa, Homo sapiens
  MOD_RES (17): Oxidized Met
  One of the Asn residues in this sequence is de-amidated
HGIQYFNNNTQHSSLFMLNEVKR

SEQ ID NO: 340 - protein, 23 aa, Homo sapiens
  MOD_RES (17): Oxidized Met
  Two of the Asn residues in this sequence are de-amidated
HGIQYFNNNTQHSSLFMLNEVKR

SEQ ID NO: 341 - protein, 15 aa, Homo sapiens
  MOD_RES (12): De-amidated Asn
VCQDCPLLAPLNDTR

SEQ ID NO: 342 - protein, 16 aa, Homo sapiens
KVCQDCPLLAPLNDTR

SEQ ID NO: 343 - protein, 16 aa, Homo sapiens
  MOD_RES (13): De-amidated Asn
KVCQDCPLLAPLNDTR

SEQ ID NO: 344 - protein, 20 aa, Homo sapiens
  MOD_RES (4): Oxidized Met
HTFMGVVSLGSPSGEVSHPR

SEQ ID NO: 345 - protein, 22 aa, Homo sapiens
AALAAFNAQNNGSNFQLEEISR

SEQ ID NO: 346 - protein, 22 aa, Homo sapiens
  One of the Asn residues in this sequence is de-amidated
AALAAFNAQNNGSNFQLEEISR

SEQ ID NO: 347 - protein, 22 aa, Homo sapiens
  One of the Asn residues in this sequence is de-amidated
AALAAFNAQNNGSNFQLEEISR

SEQ ID NO: 348 - protein, 22 aa, Homo sapiens
  Two of the Asn residues in this sequence are de-amidated
AALAAFNAQNNGSNFQLEEISR

SEQ ID NO: 349 - protein, 18 aa, Homo sapiens
AVNITSENLIDDVVSLIR

SEQ ID NO: 350 - protein, 18 aa, Homo sapiens
  One of the Asn residues in this sequence is de-amidated
AVNITSENLIDDVVSLIR

SEQ ID NO: 351 - protein, 8 aa, Homo sapiens
  MOD_RES (2): De-amidated Asn
HNSTGCLR

SEQ ID NO: 352 - protein, 11 aa, Homo sapiens
  MOD_RES (5): De-amidated Asn
EIRHNSTGCLR

SEQ ID NO: 353 - protein, 14 aa, Homo sapiens
  MOD_RES (3): De-amidated Asn
LANLTQGEDQYYLR

SEQ ID NO: 354 - protein, 14 aa, Homo sapiens
  MOD_RES (3): De-amidated Asn
LANLTQGEDQYYLR

SEQ ID NO: 355 - protein, 20 aa, Homo sapiens
  One of the Asn residues in this sequence is de-amidated
MLNTSSLLEQLNEQFNWVSR

SEQ ID NO: 356 - protein, 20 aa, Homo sapiens
  MOD_RES (1): Oxidized Met
  One of the Asn residues in this sequence is de-amidated
MLNTSSLLEQLNEQFNWVSR

SEQ ID NO: 357 - protein, 20 aa, Homo sapiens
QLEEFLNQSSPFYFWMNGDR

SEQ ID NO: 358 - protein, 20 aa, Homo sapiens
  One of the Asn residues in this sequence is de-amidated
QLEEFLNQSSPFYFWMNGDR

SEQ ID NO: 359 - protein, 20 aa, Homo sapiens
  MOD_RES (7): De-amidated Asn
  MOD_RES (16): Oxidized Met
  MOD_RES (17): De-amidated Asn
QLEEFLNQSSPFYFWMNGDR

SEQ ID NO: 360 - protein, 11 aa, Homo sapiens
  MOD_RES (3): De-amidated Asn
GLNVTLSSTGR

SEQ ID NO: 361 - protein, 16 aa, Homo sapiens
  MOD_RES (8): De-amidated Asn
FSDGLESNSSTQFEVK

SEQ ID NO: 362 - protein, 17 aa, Homo sapiens
  MOD_RES (8): De-amidated Asn
FSDGLESNSSTQFEVKK

SEQ ID NO: 363 - protein, 10 aa, Homo sapiens
ADLSGITGAR

SEQ ID NO: 364 - protein, 20 aa, Homo sapiens
  MOD_RES (2): De-amidated Asn
FNLTETSEAEIHQSFQHLLR

SEQ ID NO: 365 - protein, 23 aa, Homo sapiens
  MOD_RES (5): De-amidated Asn
GLKFNLTETSEAEIHQSFQHLLR

SEQ ID NO: 366 - protein, 9 aa, Homo sapiens
WFYIASAFR

SEQ ID NO: 367 - protein, 15 aa, Homo sapiens
QNQCFYNSSYLNVQR

SEQ ID NO: 368 - protein, 15 aa, Homo sapiens
  One of the Asn residues in this sequence is de-amidated
QNQCFYNSSYLNVQR

SEQ ID NO: 369 - protein, 15 aa, Homo sapiens
  One of the Asn residues in this sequence is de-amidated
QNQCFYNSSYLNVQR

SEQ ID NO: 370 - protein, 15 aa, Homo sapiens
  One of the Asn residues in this sequence is de-amidated
QNQCFYNSSYLNVQR

SEQ ID NO: 371 - protein, 15 aa, Homo sapiens
  One of the Asn residues in this sequence is de-amidated
WFYIASAFRNEEYNK

SEQ ID NO: 372 - protein, 22 aa, Homo sapiens
  Two of the Asn residues in this sequence are de-amidated
QNQCFYNSSYLNVQRENGTVSR

SEQ ID NO: 373 - protein, 16 aa, Homo sapiens
  MOD_RES (6): Oxidized Met
  MOD_RES (8): De-amidated Asn
HEEGHMLNCTCFGQGR

SEQ ID NO: 374 - protein, 17 aa, Homo sapiens
  MOD_RES (7): Oxidized Met
  MOD_RES (9): De-amidated Asn
RHEEGHMLNCTCFGQGR

SEQ ID NO: 375 - protein, 18 aa, Homo sapiens
  One of the Asn residues in this sequence is de-amidated
DQCIVDDITYNVNDTFHK

SEQ ID NO: 376 - protein, 20 aa, Homo sapiens
  One of the Asn residues in this sequence is de-amidated
LDAPTNLQFVNETDSTVLVR

SEQ ID NO: 377 - protein, 19 aa, Homo sapiens
  One of the Asn residues in this sequence is de-amidated
DQCIVDDITYNVNDTFHKR

SEQ ID NO: 378 - protein, 32 aa, Homo sapiens
  One of the Asn residues in this sequence is de-amidated
ESKPLTAQQTTKLDAPTNLQFVNETDSTVLVR

(19) United States
(12) Patent Application Publication
(10) Pub. No.: US 2007/0269895 A1
(43) Pub. Date: Nov. 22, 2007

(54) Methods for quantitative proteome analysis of glycoproteins

(75) Inventors: Rudolf H. Aebersold, Zurich (SZ); Hui Zhang, Baltimore, MD (US); Leroy E. Hood, Seattle, WA (US); Bingyun Sun, Seattle, WA (US)

Correspondence Address: MCDERMOTT, WILL & EMERY, 4370 LA JOLLA VILLAGE DRIVE, SUITE 700, SAN DIEGO, CA, 92122, US

(73) Assignee: The Institute for Systems Biology, Seattle, WA (US)

(21) Appl. No.: 11/586,485

(22) Filed: Oct. 24, 2006

Related U.S. Application Data
(63) Continuation-in-part of application No. 10/454,375, filed on Jun. 3, 2003, now Pat. No. 7,183,118.
(60) Provisional application No. 60/385,707, filed on Jun. 3, 2002.
(60) Provisional application No. 60/469,361, filed on May 9, 2003.

(51) Int. Cl. G01N 33/00; G01N 33/53; G01N 37/00

(52) U.S. Cl. 436/56; 435/262; 435/7.1; 436/86

(57) ABSTRACT

The invention provides a method for identifying and quantifying polyglycopeptides in a sample. The method can include the steps of immobilizing glycopolypeptides to a solid support; cleaving the immobilized glycopolypeptides, thereby releasing non-glycosylated peptides and retaining immobilized glycopeptides; releasing the glycopeptides from the solid support; and analyzing the released glycopeptides. The method can further include the step of identifying one or more glycopeptides, for example, using mass spectrometry.

This application is a continuation-in-part of U.S. application Ser. No. 10/454,375, filed Jun. 3, 2003, which claims the benefit of priority of U.S. Provisional application Ser. No. 60/385,707, filed Jun. 3, 2002, and U.S. Provisional application Ser. No. 60/469,361, filed May 9, 2003, each of which the entire contents are incorporated herein by reference.

This invention was made with government support under grant numbers 1U54DA021519, 1U54CA119347 and 1P50GM076547 awarded by National Institutes of Health. The government has certain rights in the invention.

### BACKGROUND OF THE INVENTION

The present invention relates generally to the field of proteomics and more specifically to quantitative analysis of glycoproteins.

Complete genomic sequences and large partial (EST) sequence databases potentially identify every gene in a species. However, the sequences alone do not explain the mechanism of biological and clinical processes because they do not explain how the genes and their products cooperate to carry out a specific process or function. Furthermore, the gene sequence does not predict the amount or the activity of the protein products nor does it answer the questions of whether, how, and at what position(s) a protein may be modified.

Quantitative protein profiling has been recognized as an important approach for profiling the physiological state or pathological state of cells or organisms. Specific expectations of quantitative protein profiles include the possibility to detect diagnostic and prognostic disease markers, to discover proteins as therapeutic targets or to learn about basic biological mechanisms.

Not only do the amounts and type of proteins expressed vary in different pathological states, post-translational modifications of proteins also vary depending on the physiological or pathological state of cells or organisms. Thus, it is important to be able to profile the amount and types of expressed proteins as well as protein modifications.

Glycosylation has long been recognized as the most common post-translational modification affecting the functions of proteins, such as protein stability, enzymatic activity and protein-protein interactions. Differential glycosylation is a major source of protein microheterogeneity. Glycoproteins play key roles in cell communications, signaling and cell adhesion. Changes in carbohydrates in cell surface and body fluid are demonstrated in cancer and other disease states and highlights their importance. However, studies on protein glycosylation have been complicated by the diverse structure of protein glycans and the lack of effective tools to identify the glycosylation site(s) on proteins and of glycan structures. Oligosaccharides can be linked to serine or threonine residues (O-glycosylation) or to asparagine residues (N-glycosylation), and glycoproteins can have different oligosaccharides attached to any given possible site(s).

Among the many post-translation modifications of proteins, glycosylation is a modification that is common to proteins that are exposed to an extracellular environment. For example, proteins expressed on the surface of a cell are exposed to the external environment such as blood or surrounding tissue. Similarly, proteins that are secreted from a cell, for example, into the bloodstream, are commonly glycosylated.

Among the diverse types of proteins expressed by cells, proteins that are integral to or associated with lipid membranes perform a wide range of essential cellular functions. Pores, channels, pumps and transporters facilitate the exchange of membrane impermeable molecules between cellular compartments and between the cell and its extracellular environment. Transmembrane receptors sense changes in the cellular environment and, typically via associated proteins, initiate specific intracellular responses. Cell adhesion proteins mediate cell-specific interactions with other cells and the extracellular matrix. Lipid membranes also provide a hydrophobic environment for biochemical reactions that is dramatically different from that of the cytoplasm and other hydrophilic cellular compartments.

Membrane proteins, in particular those spanning the plasma membrane, are also of considerable diagnostic and therapeutic importance, which is further reinforced due to their easy accessibility. Antisera to proteins that are selectively expressed on the surface of a specific cell type have been used extensively for the classification of cells and for their preparative isolation by fluorescent activated cell sorting or related methods. Membrane proteins, as exemplified by Her2/neu, the abundance of which is modulated in the course of certain diseases such as breast cancer, are commonly used as diagnostic indicators and, less frequently, as therapeutic targets. A humanized monoclonal antibody (Herceptin, Genentech, Palo Alto, Calif.) that specifically recognizes Her2/neu receptors is the basis for a successful therapy of breast cancer, and antibodies to other cell surface proteins are also undergoing clinical trials as anticancer agents. Moreover, the majority of current effective therapeutic agents for diseases such as hypertension and heart disease are receptor antagonists that target and selectively modify the activity of specific membrane proteins. It is therefore apparent that a general technique capable of systematically identifying membrane proteins and of accurately detecting quantitative changes in the membrane protein profiles of different cell populations or tissues would be of considerable importance for biology and for applied biomedical research.

In addition to membrane bound proteins, proteins secreted by cells or shed from the cell surface, including hormones, lymphokines, interferons, transferrin, antibodies, proteases, protease inhibitors, and other factors, perform critical functions with respect to the physiological activity of an organism. Examples of physiologically important secreted proteins include the interferons, lymphokines, protein and peptide hormones. Aberrant availability of such proteins can have grave clinical consequences. It is therefore apparent that the ability to precisely quantitatively profile secreted proteins would be of great importance for the discovery of the mechanisms regulating a wide variety of physiological processes in health and disease and for diagnostic or prognostic purposes. Such secreted proteins are present in body fluids such as blood serum and plasma, cerebrospinal fluid, urine, lung lavage, breast milk, pancreatic juice, and saliva. For example, the presence of increased levels of prostate-specific antigen has been used as a diagnostic marker for prostate cancer. Furthermore, the use of agonists or antagonists or the replacement of soluble secreted proteins is an important mode of therapy for a wide range of diseases.

Quantitative proteomics requires the analysis of complex protein samples. In the case of clinical diagnosis, the ability to obtain appropriate specimens for clinical analysis is important for ease and accuracy of diagnosis. As discussed above, a number of biologically important molecules are secreted and are therefore present in body fluids such as blood and serum, cerebrospinal fluid, saliva, and the like. In addition to the presence of important biological molecules, body fluids also provide an attractive specimen source because body fluids are generally readily accessible and available in reasonable quantities for clinical analysis. It is therefore apparent that a general method for the quantitative analysis of the proteins contained in body fluids in health and disease would be of great diagnostic and clinical importance.

A key problem with the proteomic analysis of serum and many other body fluids is the peculiar protein composition of these specimens. The protein composition is dominated by a few proteins that are extraordinarily abundant, with albumin alone representing 50% of the total plasma proteins. Due to the abundance of these major proteins as well as the presence of multiple modified forms of these abundant proteins, the large number of protein species of lower abundance are obscured or inaccessible by traditional proteomics analysis methods such as two-dimensional electrophoresis (2DE).

The classes of proteins described above, membrane proteins, secreted proteins, and proteins in body fluids have in common that they have a high propensity for being glycosylated, that is, modified post translationally with a carbohydrate structure of varying complexity at one or several amino acid residues. Thus, the analysis of glycoproteins allows characterization of important biological molecules.

Thus, there exists a need for methods of high throughput and quantitative analysis of glycoproteins and glycoprotein profiling. The present invention satisfies this need and provides related advantages as well.

### SUMMARY OF INVENTION

The invention provides a method for identifying and quantifying polyglycopeptides in a sample. The method can include the steps of immobilizing glycopolypeptides to a solid support; cleaving the immobilized glycopolypeptides, thereby releasing non-glycosylated peptides and retaining immobilized glycopeptides; releasing the glycopeptides from the solid support; and analyzing the released glycopeptides. The method can further include the step of identifying one or more glycopeptides, for example, using mass spectrometry.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic diagram of an exemplary method of identifying and quantifying glycopolypeptides/glycoproteins and for determining quantitative changes in the glycosylation state of proteins.

FIG. 2 shows oxidation of a carbohydrate to an aldehyde followed by covalent coupling to hydrazide beads.

FIG. 3 shows representative chemical reagents that have been tested and proved to be able to label amino groups of glycopeptides. The structures of labeled peptide are listed in the right column.

FIG. 4 shows total protein staining or glycoprotein staining of crude serum before (−) and after immobilization (+) of glycoproteins to hydrazide resin. Proteins were separated by SDS-PAGE and stained with silver (left) or Gel Code Blue glycoprotein staining reagent (right).

FIG. 5 shows an outline and comparison of the results of glycopeptide analysis of serum proteins observed with three methods: cysteine capture with extensive separation, glycopeptide capture and single liquid chromatography-mass spectrometry/mass spectrometry (LC-MS/MS), and cysteine capture and single LC-MS/MS.

FIG. 6 shows identification of glycosylated proteins secreted from macrophages. Glycoproteins were identified from secreted proteins of untreated or LPS-treated RAW macrophage cells.

FIG. 7 shows comparison of protein/peptide identification from the microsomal fraction of the prostate cancer cell line LNCaP using an ICAT™ reagent or selective isolation of N-glycosylated peptides.

FIG. 8 shows subcellular location of glycoproteins identified from a crude microsomal fraction of LNCaP prostate epithelial cells.

FIG. 9 shows the chemistry and schematic diagram of isotopically labeling the N-termini of the immobilized glycopeptides by attaching differentially isotopically labeled forms of the amino acid phenylalanine (Phe) to their N-termini.

FIG. 10 shows isotopic labeling with Phe and identification of glycopeptides (SEQ ID NOS:1-10) using MS/MS. The glycopeptides were isolated from 1 μl of mouse ascites fluid.

FIG. 11 shows collision-induced dissociation (CID) spectrum of one of the peptides (SEQ ID NO:7) identified in FIG. 10 (circled).

FIG. 12 shows reconstructed ion chromatograms for the peptide measured in FIG. 11. The ratio of the calculated peak area for the heavy and light form of the isotope tagged peptides was used to determine the relative peptide abundance in the original mixtures.

FIG. 13 shows the quantification for a single peptide pair. A single scan of the mass spectrometer at spot 28 from a MALDI plate in MS mode identified eight paired signals with a mass difference of four units (indicated with *).

FIG. 14 shows analysis of a precursor ion by MS/MS. Sequence database searching of the resulting spectrum identified the peptide sequence as IYSGILN#LSDITK from human plasma kallikrein, a serum protease. N# indicates the modified asparagine in the peptide sequence.

FIG. 15 shows the patterns of aligned sequences. For each position in the aligned sequence, the height of each letter is proportional to its frequency, and the most common one is on top. There was high preference of N at position 21 (removed to show the detail of other positions). The preference of N was followed by S or T at position 23 (removed to show residues in other positions).

FIG. 16 shows proteins identified from extracellular matrix of normal and prostate cancer tissues.

FIG. 17 shows the total peptides present in a single LC-MS/MS run (black dots) and the identified peptides (red dots) by CID acquired during the LC-MS/MS run followed by a search using SEQUEST.

FIG. 18 shows a schematic diagram of the strategy used to profile glycopeptides present in serum and identify biomarkers.

FIG. 19 shows the signal intensity of peptides during the elution of an LC-MS/MS run. N1 and N2 were from normal mouse serum, and T1 and T2 were glycopeptides from mouse serum with skin cancer.

FIG. 20 shows the intensity of deconvoluted peptides during different elution time from serum of normal mice and mice with skin cancer. The left panel shows peptides in normal mouse. The right panel shows peptides in cancer mouse.

FIG. 21 shows normalized peptide abundance between cancer and normal mouse. The relative peptide intensity of cancer mouse to normal mouse.

FIG. 22 shows clustering analysis of normal mice and mice with cancer. Automatic, whole feature clustering of mouse serum distinguishes cancer from healthy. All the cancer mice clustered together (indicated as 11A, 12A, 13A in experiment one, upper panel; and M11, M12, M13 in experiment two, lower panel).

FIG. 23 shows clustering analysis of samples from individuals before and after overnight fasting. Automatic clustering of serum from three individuals before and after overnight fasting consistently separates individuals (experiment one, upper panel; experiment two, lower panel). Serum samples from the same person cluster together.

FIG. 24 shows a schematic diagram of a glycosylation occupancy study of serum from congenital disorders of glycosylation (CDG) patients.

FIG. 25 shows a schematic diagram of a study on total level of glycosylation using serum from obese and normal mice.

FIG. 26 shows sequences of heavy isotope labeled synthetic peptide standards (SEQ ID NOS:11-19) identified by mass-spectrometry. V* is the heavy valine and F# is the heavy phenylalanine.

FIG. 27 shows peptides (SEQ ID NOS:20-29) identified from a series of enzymatic cleavages to release O-linked glycopeptides from hydrazide resin after N-linked glycopeptides were released.

FIG. 28 shows identified N-linked glycopeptides (SEQ ID NOS:30-48), with the consensus NXT/S motif highlighted.

FIG. 29 shows peptides (SEQ ID NOS:49-63) identified with O-linked oligosaccharides. These were generated by the removal of the O-linked oligosaccharide chains in the electrospray source. The site of carbohydrate attachment is characterized by a loss of water at Ser or Thr to which the O-linked oligosaccharides were linked. The serine or threonine residues with the 18 Dalton water loss are circled.

FIGS. 30A-30C show a schematic illustration of glyco-protein capture (FIGS. 30A and 30B) and glyco-peptide capture (FIG. 30C). FIG. 30A shows captured glycoprotein on a polymeric support. FIG. 30B shows captured glycopeptide after on-support of the glycoprotein proteolysis and wash steps. FIG. 30C shows the strategy of glyco-peptide capture. Proteins are denatured and all the glycosylation sites are exposed. Then, the denatured proteins are digested into peptides through proteolysis. Glycopeptides are coupled to a polymeric support through hydrazide chemistry, and the non-glycosylated peptides are washed away. Finally, the captured peptides are liberated and subjected to mass spectrometry (MS) analysis.

FIGS. 31A and 31B show the results from the MALDI-TOF analysis of the glyco-peptide capture strategy applied to tryptic peptides of chicken avidin. FIG. 31A shows deglycosylated N-glycopeptides captured from chicken avidin. FIG. 31B shows non-captured tryptic peptides from avidin after sequential glycopeptide capture and deglycosylation by PNGase F. The inset shows the expanded m/z region from 1770 to 1955 of FIG. 31B.

FIG. 32 shows a peptide3D image of N-glycopeptides detected by LTQ nanoLC-MS/MS from microsomal fraction of cisplatin-resistant ovarian cancer cell line, IGROV-I/CP. A gradient of 10-35% solvent B (100% acetonitrile) over 30 minutes was applied to a 75 μm×10 cm fused silica capillary column packed with 100 Å pore-size Magic C18AQ™ material. Eluting peptides were analyzed by nanoLC-MS and data dependent acquisition, selecting 3 precursor ions for MS/MS with a dynamic exclusion setting of 1. The peptides that were identified by the SEQUEST™ database searching with a peptide probability score above 0.9 are displayed. The color indicates different probability values.

FIG. 33 shows molecular function of glycoproteins identified from a crude microsomal fraction of cisplatin-resistant ovarian cancer cell line, IGROV-1. The results are obtained from GoMiner, and a total of 302 proteins from two biological replicates and 4 LTQ nanoLCMS-MS runs are presented. Some proteins are represented in more than one category.

FIG. 34 shows comparison of proteins identified using the glycopeptide capture approach (red) and the ICAT approach (green) applied to the microsomal fraction of the IGROV-1/CP cell line, and from the glycoprotein capture approach to the microsomal fraction of the LNCaP cell line (yellow).

FIG. 35 shows LC-MALDI analysis of glycoprotein versus glycopeptide capture of fetuin.

FIG. 36 shows glycopeptides identified by capture methods disclosed herein.

### DETAILED DESCRIPTION OF THE INVENTION

The invention provides methods for quantitative profiling of glycoproteins and glycopeptides on a proteome-wide scale. The methods of the invention allow the identification and quantification of glycoproteins in a complex sample and determination of the sites of glycosylation. The methods of the invention can be used to determine changes in the abundance of glycoproteins and changes in the state of glycosylation at individual glycosylation sites on those glycoproteins that occur in response to perturbations of biological systems and organisms in health and disease.

The methods of the invention can be used to purify glycosylated proteins or peptides and identify and quantify the glycosylation sites. Because the methods of the invention are directed to isolating glypolypeptides, the methods also reduce the complexity of analysis since many proteins and fragments of glycoproteins do not contain carbohydrate. This can simplify the analysis of complex biological samples such as serum (see below). The methods of the invention are advantageous for the determination of protein glycosylation in glycome studies and can be used to isolate and identify glycoproteins from cell membrane or body fluids to determine specific glycoprotein changes related to certain disease states or cancer. The methods of the invention can be used for detecting quantitative changes in protein samples containing glycoproteins and to detect their extent of glycosylation. The methods of the invention are applicable for the identification and/or characterization of diagnostic biomarkers, immunotherapy, or other diagnositic or therapeutic applications. The methods of the invention can also be used to evaluate the effectiveness of drugs during drug development, optimal dosing, toxicology, drug targeting, and related therapeutic applications.

In one embodiment, the cis-diol groups of carbohydrates in glycoproteins can be oxidized by periodate oxidation to give a di-aldehyde, which is reactive to a hydrazide gel with an agarose support to form covalent hydrazone bonds. The immobilized glycoproteins are subjected to protease digestion followed by extensive washing to remove the non-glycosylated peptides. The immobilized glycopeptides are released from beads by chemicals or glycosidases. The isolated peptides are analyzed by mass spectrometry (MS), and the glycopeptide sequence and corresponding proteins are identified by MS/MS combined with a database search. The glycopeptides can also be isotopically labeled, for example, at the amino or carboxyl termini to allow the quantities of glycopeptides from different biological samples to be compared.

The methods of the invention are based on selectively isolating glycosylated peptides, or peptides that were glycosylated in the original protein sample, from a complex sample. The sample consists of peptide fragments of proteins generated, for example, by enzymatic digestion or chemical cleavage. A stable isotope tag is introduced into the isolated peptide fragments to facilitate mass spectrometric analysis and accurate quantification of the peptide fragments.

The invention provides a method for identifying and quantifying glycopolypeptides in a sample. The method can include the steps of derivatizing glycopolypeptides in a polypeptide sample, for example, by oxidation; immobilizing the derivatized glycopolypeptides to a solid support; cleaving the immobilized glycopolypeptides, thereby releasing non-glycosylated peptide fragments and retaining immobilized glycopeptide fragments; optionally labeling the immobilized glycopeptide fragments with an isotope tag; releasing the glycopeptide fragments from the solid support, thereby generating released glycopeptide fragments; analyzing the released glycopeptide fragments or their de-glycosylated counterparts using mass spectrometry; and quantifying the amount of the identified glycopeptide fragment. The released glycopolypeptides can be released with the carbohydrate still attached (the glycosylated form) or with the carbohydrate removed (the de-glycosylated form).

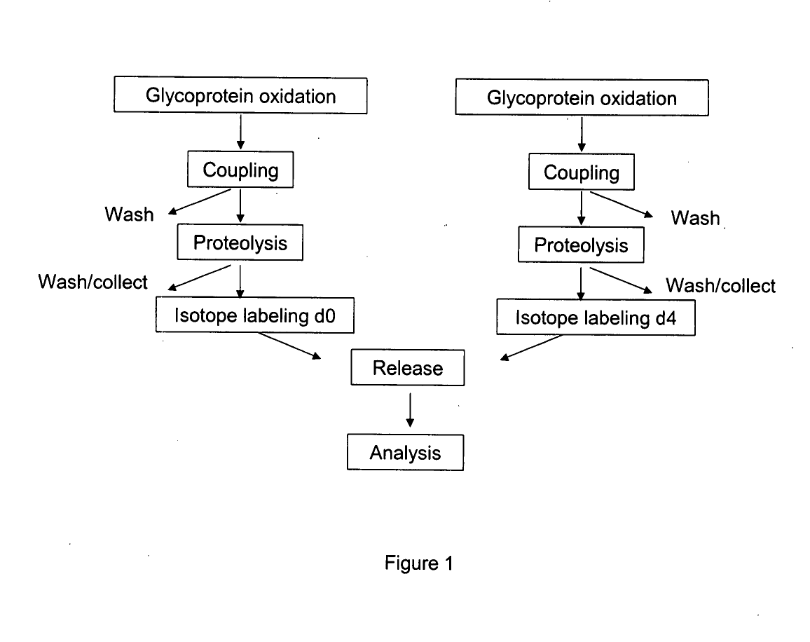

An embodiment of the present invention is depicted in FIG. 1. A sample containing glycopolypeptides is chemically modified so that carbohydrates of the glycopolypeptides in the sample can be selectively bound to a solid support. For example, the glycopolypeptides can be bound covalently to a solid support by chemically modifying the carbohydrate so that the carbohydrate can covalently bind to a reactive group on a solid support. In the embodiment depicted in FIG. 1, the carbohydrates of the sample glycopolypeptides are oxidized. The carbohydrate can be oxidized, for example, to aldehydes. The oxidized moiety, such as an aldehyde moiety, of the glycopolypeptides can react with a solid support containing hydrazide or amine moieties, allowing covalent attachment of glycosylated polypeptides to a solid support via hydrazine chemistry. The sample glycopolypeptides are immobilized through the chemically modified carbohydrate, for example, the aldehyde, allowing the removal of non-glycosylated sample proteins by washing of the solid support. If desired, the immobilized glycopolypeptides can be denatured and/or reduced. The immobilized glycopolypeptides are cleaved into fragments using either protease or chemical cleavage. Cleavage results in the release of peptide fragments that do not contain carbohydrate and are therefore not immobilized. These released non-glycosylated peptide fragments optionally can be further characterized, if desired.

Following cleavage, glycosylated peptide fragments (glycopeptide fragments) remain bound to the solid support. To facilitate quantitative mass spectrometry (MS) analysis, immobilized glycopeptide fragments can be isotopically labeled. If it is desired to characterize most or all of the immobilized glycopeptide fragments, the isotope tagging reagent contains an amino or carboxyl reactive group so that the N-terminus or C-terminus of the glycopeptide fragments can be labeled (see FIGS. 1, 3 and 9). The immobilized glycopeptide fragments can be cleaved from the solid support chemically or enzymatically, for example, using glycosidases such as N-glycanase (N-glycosidase) or O-glycanase (O-glycosidase). The released glycopeptide fragments or their deglycosylated forms can be analyzed, for example, using MS.

As used herein, the term “polypeptide” refers to a peptide or polypeptide of two or more amino acids. A polypeptide can also be modified by naturally occurring modifications such as post-translational modifications, including phosphorylation, fatty acylation, prenylation, sulfation, hydroxylation, acetylation, addition of carbohydrate, addition of prosthetic groups or cofactors, formation of disulfide bonds, proteolysis, assembly into macromolecular complexes, and the like. A “peptide fragment” is a peptide of two or more amino acids, generally derived from a larger polypeptide.

As used herein, a “glycopolypeptide” or “glycoprotein” refers to a polypeptide that contains a covalently bound carbohydrate group. The carbohydrate can be a monosaccharide, oligosaccharide or polysaccharide. Proteoglycans are included within the meaning of “glycopolypeptide.” A glycopolypeptide can additionally contain other post-translational modifications. A “glycopeptide” refers to a peptide that contains covalently bound carbohydrate. A “glycopeptide fragment” refers to a peptide fragment resulting from enzymatic or chemical cleavage of a larger polypeptide in which the peptide fragment retains covalently bound carbohydrate. It is understood that a glycopeptide fragment or peptide fragment refers to the peptides that result from a particular cleavage reaction, regardless of whether the resulting peptide was present before or after the cleavage reaction. Thus, a peptide that does not contain a cleavage site will be present after the cleavage reaction and is considered to be a peptide fragment resulting from that particular cleavage reaction. For example, if bound glycopeptides are cleaved, the resulting cleavage products retaining bound carbohydrate are considered to be glycopeptide fragments. The glycosylated fragments can remain bound to the solid support, and such bound glycopeptide fragments are considered to include those fragments that were not cleaved due to the absence of a cleavage site.

As disclosed herein, a glycopolypeptide or glycopeptide can be processed such that the carbohydrate is removed from the parent glycopolypeptide. It is understood that such an originally glycosylated polypeptide is still referred to herein as a glycopolypeptide or glycopeptide even if the carbohydrate is removed enzymatically and/or chemically. Thus, a glycopolypeptide or glycopeptide can refer to a glycosylated or de-glycosylated form of a polypeptide. A glycopolypeptide or glycopeptide from which the carbohydrate is removed is referred to as the de-glycosylated form of a polypeptide whereas a glycopolypeptide or glycopeptide which retains its carbohydrate is referred to as the glycosylated form of a polypeptide.

As used herein, the term “sample” is intended to mean any biological fluid, cell, tissue, organ or portion thereof, that includes one or more different molecules such as nucleic acids, polypeptides, or small molecules. A sample can be a tissue section obtained by biopsy, or cells that are placed in or adapted to tissue culture. A sample can also be a biological fluid specimen such as blood, serum or plasma, cerebrospinal fluid, urine, saliva, seminal plasma, pancreatic juice, breast milk, lung lavage, and the like. A sample can additionally be a cell extract from any species, including prokaryotic and eukaryotic cells as well as viruses. A tissue or biological fluid specimen can be further fractionated, if desired, to a fraction containing particular cell types.

As used herein, a “polypeptide sample” refers to a sample containing two or more different polypeptides. A polypeptide sample can include tens, hundreds, or even thousands or more different polypeptides. A polypeptide sample can also include non-protein molecules so long as the sample contains polypeptides. A polypeptide sample can be a whole cell or tissue extract or can be a biological fluid. Furthermore, a polypeptide sample can be fractionated using well known methods, as disclosed herein, into partially or substantially purified protein fractions.

The use of biological fluids such as a body fluid as a sample source is particularly useful in methods of the invention. Biological fluid specimens are generally readily accessible and available in relatively large quantities for clinical analysis. Biological fluids can be used to analyze diagnostic and prognostic markers for various diseases. In addition to ready accessibility, body fluid specimens do not require any prior knowledge of the specific organ or the specific site in an organ that might be affected by disease. Because body fluids, in particular blood, are in contact with numerous body organs, body fluids “pick up” molecular signatures indicating pathology due to secretion or cell lysis associated with a pathological condition. Body fluids also pick up molecular signatures that are suitable for evaluating drug dosage, drug targets and/or toxic effects, as disclosed herein.

Quantitative proteomics, defined as the comparison of relative protein changes in different proteomes, has been recognized as an important component of the emerging science of functional genomics. The technology is expected to facilitate the detection and identification of diagnostic or prognostic disease markers, the discovery of proteins as therapeutic targets and to provide new functional insights into biological processes. Two methods have been used preferentially to generate quantitative profiles of complex protein mixtures. The first and most commonly used is a combination of two-dimensional gel electrophoresis (2DE) and mass spectrometry (MS). The second is a more recently developed technique based on stable isotope tagging of proteins and automated peptide tandem mass spectrometry (Oda et al., Proc. Natl. Acad. Sci. USA 96:6591-6596 (1999); Veenstra et al., J. Am. Soc. Mass. Spectrom. 11:78-82 (2000); Gygi et al., Nat. Biotechnol. 17:994-999 (1999)). To date, neither method has succeeded in determining the complete proteome of any species. This is mainly due to the “top down” mode of operation of either method in which the most abundant proteins are preferentially or exclusively analyzed.

Given the complexities of global proteome analysis, several studies have adopted a “divide and conquer” strategy to handle the “top down” problem by comprehensively analyzing specific subsets of the proteome that are selectively isolated. Such studies include the analysis of functional multiprotein complexes such as the ribosome (Link et al., Nat. Biotechnol. 17:676-682 (1999)), spliceosome (Rappsilber et al., Genome Res. 12:1231-1245 (2002); Zhou et al., Nature 419:182-185 (2002)), and nuclear pore complex (Rout et al., J. Cell Biol. 148:635-651 (2000)), or organelles, such as mitochondria (Fountoulakis et al., Electrophoresis 23:311-328 (2002)), peroxisomes (Yi et al., Electrophoresis 23:3205-3216 (2002)), microsomes (Han et al., Nat. Biotechnol. 19:946-951 (2001)) and nuclei (Bergquist et al., J. Neurosci. Methods 109:3-11 (2001)). Alternatively, proteins that contain common distinguishing structural features, such as phosphate ester groups ((Ficarro et al., Nat. Biotechnol. 20:301-305 (2002); Oda et al., Nat. Biotechnol. 19:379-382 (2001); Zhou et al., Nat. Biotechnol. 19:375-378 (2001)), cysteine residues (Gygi et al. supra (1999); Spahr et al., Electrophoresis 21:1635-1650 (2000)) or have the ability to specifically bind to certain compounds (Haystead et al., Eur. J. Biochem. 214:459-467 (1993); Adam et al., Nat. Biotechnol. 20:805-809 (2002)) have been selectively enriched prior to MS analysis. These strategies have in common that they focus on the in-depth analysis of sub-proteomes of rich biological context, thus minimizing the repeated analyses of abundantly expressed proteins.

The methods of the invention utilize the selective isolation of glycopolypeptides coupled with chemical modification to facilitate MS analysis. Proteins are glycosylated by complex enzymatic mechanisms, typically at the side chains of serine or threonine residues (O-linked) or the side chains of asparagine residues (N-linked). N-linked glycosylation sites generally fall into a sequence motif that can be described as N-X-S/T, where X can be any amino acid except proline. Glycosylation plays an important function in many biological processes (reviewed in Helenius and Aebi, Science 291:2364-2369 (2001); Rudd et al., Science 291:2370-2375 (2001)).

Protein glycosylation has long been recognized as a very common post-translational modification. As discussed above, carbohydrates are linked to serine or threonine residues (O-linked glycosylation) or to asparagine residues (N-linked glycosylation) (Varki et al. Essentials of Glycobiologv Cold Spring Harbor Laboratory (1999)). Protein glycosylation, and in particular N-linked glycosylation, is prevalent in proteins destined for extracellular environments (Roth, Chem. Rev. 102:285-303 (2002)). These include proteins on the extracellular side of the plasma membrane, secreted proteins, and proteins contained in body fluids, for example, blood serum, cerebrospinal fluid, urine, breast milk, saliva, lung lavage fluid, pancreatic juice, and the like. These also happen to be the proteins in the human body that are most easily accessible for diagnostic and therapeutic purposes.

Due to the ready accessibility of body fluids exposed to the extracellular surface of cells and the presence of secreted proteins in these fluids, many clinical biomarkers and therapeutic targets are glycoproteins. These include Her2/neu in breast cancer, human chorionic gonadotropin and a-fetoprotein in germ cell tumors, prostate-specific antigen in prostate cancer, and CA125 in ovarian cancer. The Her2/neu receptor is also the target for a successful immunotherapy of breast cancer using the humanized monoclonal antibody Herceptin (Shepard et al., J. Clin. Immunol. 11:117-127 (1991)). In addition, changes in the extent of glycosylation and the carbohydrate structure of proteins on the cell surface and in body fluids have been shown to correlate with cancer and other disease states, highlighting the clinical importance of this modification as an indicator or effector of pathologic mechanisms (Durand and Seta, Clin. Chem. 46:795-805 (2000); Freeze, Glycobiology I 1:129R-143R (2001); Spiro, Glycobiology 12:43R-56R (2002)). Therefore, a method for the systematic and quantitative analysis of glycoproteins would be of significance for the detection of new potential diagnostic markers and therapeutic targets.

Disclosed herein is a method for quantitative glycoprotein profiling. In one embodiment, the method is based on the conjugation of glycoproteins to a solid support using hydrazide chemistry, stable isotope labeling of glycopeptides, and the specific release of formerly N-linked glycosylated peptides via Peptide-N-Glycosidase F (PNGase F). The recovered peptides are then identified and quantified by tandem mass spectrometry (MS/MS). The method was applied to the analysis of cell surface and serum proteins, as disclosed herein.

To selectively isolate glycopolypeptides, the methods utilize chemistry and/or binding interactions that are specific for carbohydrate moieties. Selective binding of glycopolypeptides refers to the preferential binding of glycopolypeptides over non-glycosylated peptides, as demonstrated in Example II. The methods of the invention can utilize covalent coupling of glycopolypeptides, which is particularly useful for increasing the selective isolation of glycopolypeptides by allowing stringent washing to remove non-specifically bound, non-glycosylated polypeptides.

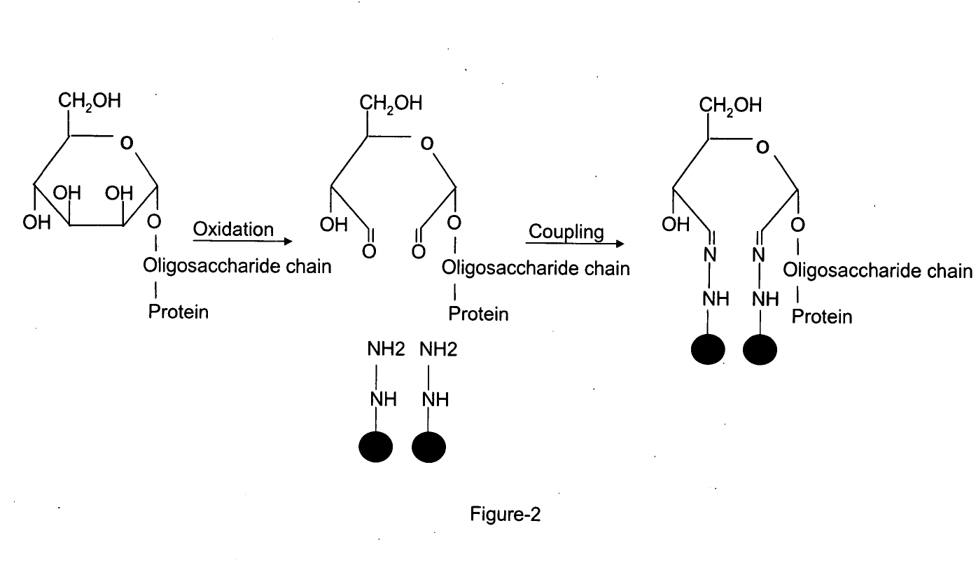

The carbohydrate moieties of a glycopolypeptide are chemically or enzymatically modified to generate a reactive group that can be selectively bound to a solid support having a corresponding reactive group. In the embodiment depicted in FIG. 2, the carbohydrates of glycopolypeptides are oxidized to aldehydes. The oxidation can be performed, for example, with sodium periodate. The hydroxyl groups of a carbohydrate can also be derivatized by epoxides or oxiranes, alkyl halogen, carbonyldiimidazoles, N,N′-disuccinimidyl carbonates, N-hydroxycuccinimidyl chloroformates, and the like. The hydroxyl groups of a carbohydrate can also be oxidized by enzymes to create reactive groups such as aldehyde groups. For example, galactose oxidase oxidizes terminal galactose or N-acetyl-D-galactose residues to form C-6 aldehyde groups. These derivatized groups can be conjugated to amine- or hydrazide- containing moieties.

The oxidation of hydroxyl groups to aldehyde using sodium periodate is specific for the carbohydrate of a glycopeptide. Sodium periodate can oxidize hydroxyl groups on adjacent carbon atoms, forming an aldehyde for coupling with amine- or hydrazide-containing molecules. Sodium periodate also reacts with hydroxylamine derivatives, compounds containing a primary amine and a secondary hydroxyl group on adjacent carbon atoms. This reaction is used to create reactive aldehydes on N-terminal serine residues of peptides. A serine residue is rare at the N-terminus of a protein. The oxidation to an aldehyde using sodium periodate is therefore specific for the carbohydrate groups of a glycopolypeptide.

Once the carbohydrate of a glycopolypeptide is modified, for example, by oxidition to aldehydes, the modified carbohydrates can bind to a solid support containing hydrazide or amine moieties, such as the hydrazide resin depicted in FIG. 2. Although illustrated with oxidation chemistry and coupling to hydrazide, it is understood that any suitable chemical modifications and/or binding interactions that allows specific binding of the carbohydrate moieties of a glycopolypeptide can be used in methods of the invention. The binding interactions of the glycopolypeptides with the solid support are generally covalent, although non-covalent interactions can also be used so long as the glycopolypeptides or glycopeptide fragments remain bound during the digestion, washing and other steps of the methods.

The methods of the invention can also be used to select and characterize subgroups of carbohydrates. Chemical modifications or enzymatic modifications using, for example, glycosidases can be used to isolate subgroups of carbohydrates. For example, the concentration of sodium periodate can be modulated so that oxidation occurs on sialic acid groups of glycoproteins. In particular, a concentration of about 1 mM of sodium periodate at 0° C. can be used to essentially exclusively modify sialic acid groups.

Glycopolypeptides containing specific monosaccharides can be targeted using a selective sugar oxidase to generate aldehyde functions, such as the galactose oxidase described above or other sugar oxidases. Furthermore, glycopolypeptides containing a subgroup of carbohydrates can be selected after the glycopolypeptides are bound to a solid support. For example, glycopeptides bound to a solid support can be selectively released using different glycosidases having specificity for particular monosaccharide structures.

The glycopolypeptides are isolated by binding to a solid support. The solid support can be, for example, a bead, resin, membrane or disk, or any solid support material suitable for methods of the invention. An advantage of using a solid support to bind the glycopolypeptides is that it allows extensive washing to remove non-glycosylated polypeptides. Thus, in the case of complex samples containing a multitude of polypeptides, the analysis can be simplified by isolating glycopolypeptides and removing the non-glycosylated polypeptides, thus reducing the number of polypeptides to be analyzed.

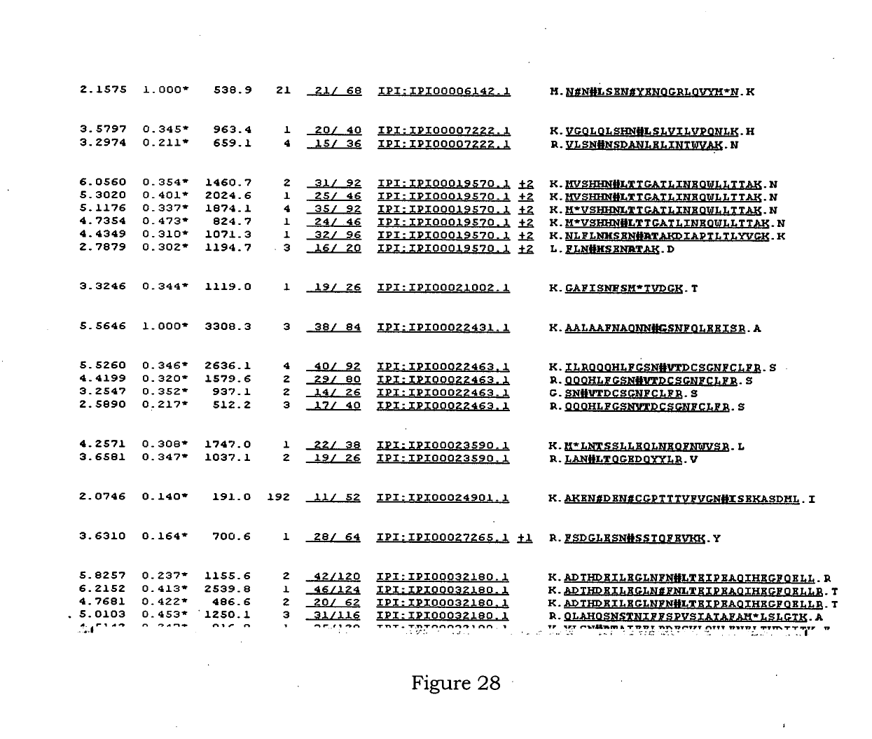

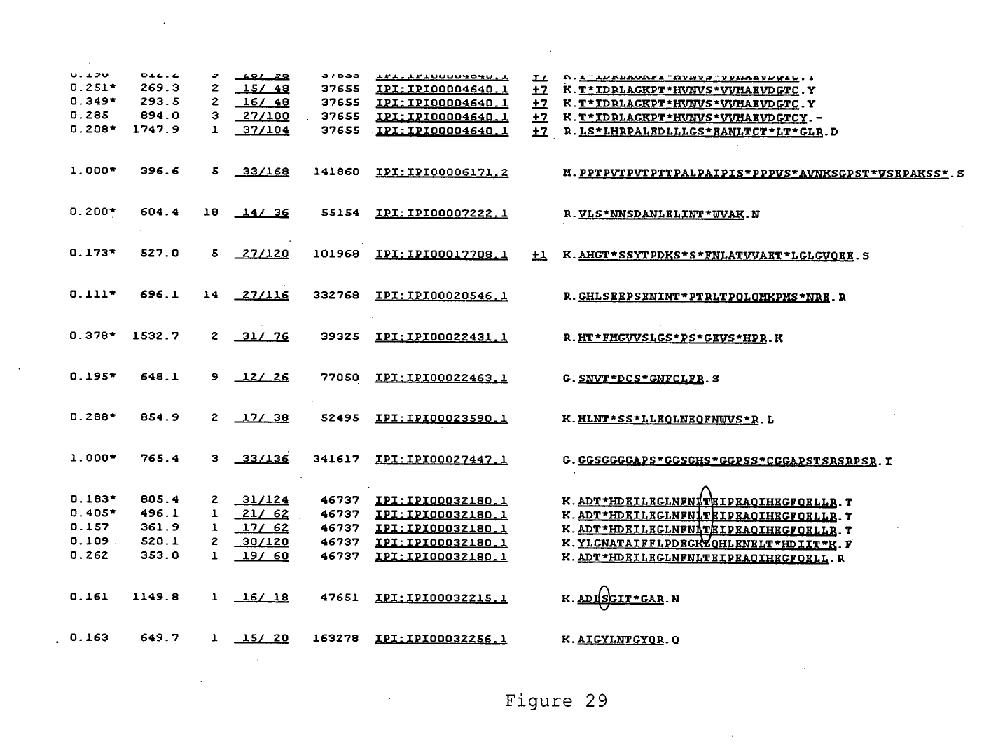

The glycopolypeptides can also be conjugated to an affinity tag through an amine group, such as biotin hydrazide. The affinity tagged glycopeptides can then be immobilized to the solid support, for example, an avidin or streptavidin solid support, and the non-glycosylated peptides are removed. The glycopeptides immobilized on the solid support can be cleaved by a protease, and the non-glycosylated peptide fragments can be removed by washing. The tagged glycopeptides can be released from the solid support by enzymatic or chemical cleavage. Alternatively, the tagged glycopeptides can be released from the solid support with the oligosaccharide and affinity tag attached (see Example XV and FIGS. 28 and 29).

Another advantage of binding the glycopolypeptides to the solid support is that it allows further manipulation of the sample molecules without the need for additional purification steps that can result in loss of sample molecules. For example, the methods of the invention can involve the steps of cleaving the bound glycopolypeptides as well as adding an isotope tag, or other desired modifications of the bound glycopolypeptides. Because the glycopolypeptides are bound, these steps can be carried out on solid phase while allowing excess reagents to be removed as well as extensive washing prior to subsequent manipulations.

The bound glycopolypeptides can be cleaved into peptide fragments to facilitate MS analysis. Thus, a polypeptide molecule can be enzymatically cleaved with one or more proteases into peptide fragments. Exemplary proteases useful for cleaving polypeptides include trypsin, chymotrypsin, pepsin, papain, Staphylococcus aureus (V8) protease, Submaxillaris protease, bromelain, thermolysin, and the like. In certain applications, proteases having cleavage specificities that cleave at fewer sites, such as sequence-specific proteases having specificity for a sequence rather than a single amino acid, can also be used, if desired. Polypeptides can also be cleaved chemically, for example, using CNBr, acid or other chemical reagents. A particularly useful cleavage reagent is the protease trypsin. One skilled in the art can readily determine appropriate conditions for cleavage to achieve a desired efficiency of peptide cleavage.

Cleavage of the bound glycopolypeptides is particularly useful for MS analysis in that one or a few peptides are generally sufficient to identify a parent polypeptide. However, it is understood that cleavage of the bound glycopolypeptides is not required, in particular where the bound glycopolypeptide is relatively small and contains a single glycosylation site. Furthermore, the cleavage reaction can be carried out after binding of glycopolypeptides to the solid support, allowing characterization of non-glycosylated peptide fragments derived from the bound glycopolypeptide. Alternatively, the cleavage reaction can be carried out prior to addition of the glycopeptides to the solid support. One skilled in the art can readily determine the desirability of cleaving the sample polypeptides and an appropriate point to perform the cleavage reaction, as needed for a particular application of the methods of the invention.

If desired, the bound glycopolypeptides can be denatured and optionally reduced. Denaturing and/or reducing the bound glycopolypeptides can be useful prior to cleavage of the glycopolypeptides, in particular protease cleavage, because this allows access to protease cleavage sites that can be masked in the native form of the glycopolypeptides. The bound glycopeptides can be denatured with detergents and/or chaotropic agents. Reducing agents such as β-mercaptoethanol, dithiothreitol, tris-carboxyethylphosphine (TCEP), and the like, can also be used, if desired. As discussed above, the binding of the glycopolypeptides to a solid support allows the denaturation step to be carried out followed by extensive washing to remove denaturants that could inhibit the enzymatic or chemical cleavage reactions. The use of denaturants and/or reducing agents can also be used to dissociate protein complexes in which non-glycosylated proteins form complexes with bound glycopolypeptides. Thus, the use of these agents can be used to increase the specificity for glycopolypeptides by washing away non-glycosylated polypeptides from the solid support.

Treatment of the bound glycopolypeptides with a cleavage reagent results in the generation of peptide fragments. Because the carbohydrate moiety is bound to the solid support, those peptide fragments that contain the glycosylated residue remain bound to the solid support. Following cleavage of the bound glycopolypeptides, glycopeptide fragments remain bound to the solid support via binding of the carbohydrate moiety. Peptide fragments that are not glycosylated are released from the solid support. If desired, the released non-glycosylated peptides can be analyzed, as described in more detail below.

The methods of the invention can be used to identify and/or quantify the amount of a glycopolypeptide present in a sample. A particularly useful method for identifying and quantifying a glycopolypeptide is mass spectrometry (MS). The methods of the invention can be used to identify a glycopolypeptide qualitatively, for example, using MS analysis. If desired, an isotope tag can be added to the bound glycopeptide fragments, in particular to facilitate quantitative analysis by MS.

As used herein an “isotope tag” refers to a chemical moiety having suitable chemical properties for incorporation of an isotope, allowing the generation of chemically identical reagents of different mass which can be used to differentially tag a polypeptide in two samples. The isotope tag also has an appropriate composition to allow incorporation of a stable isotope at one or more atoms. A particularly useful stable isotope pair is hydrogen and deuterium, which can be readily distinguished using mass spectrometry as light and heavy forms, respectively. Any of a number of isotopic atoms can be incorporated into the isotope tag so long as the heavy and light forms can be distinguished using mass spectrometry, for example, ¹³C, ¹⁵N, ¹⁷O, ¹⁸O or ³⁴S. Exemplary isotope tags include the 4,7,10-trioxa-1,13-tridecanediamine based linker and its related deuterated form, 2,2′,3,3′,11,11′,12,12′-octadeutero-4,7,10-trioxa-1,13-tridecanediamine, described by Gygi et al. (Nature Biotechnol. 17:994-999 (1999). Other exemplary isotope tags have also been described previously (see WO 00/11208, which is incorporated herein by reference).

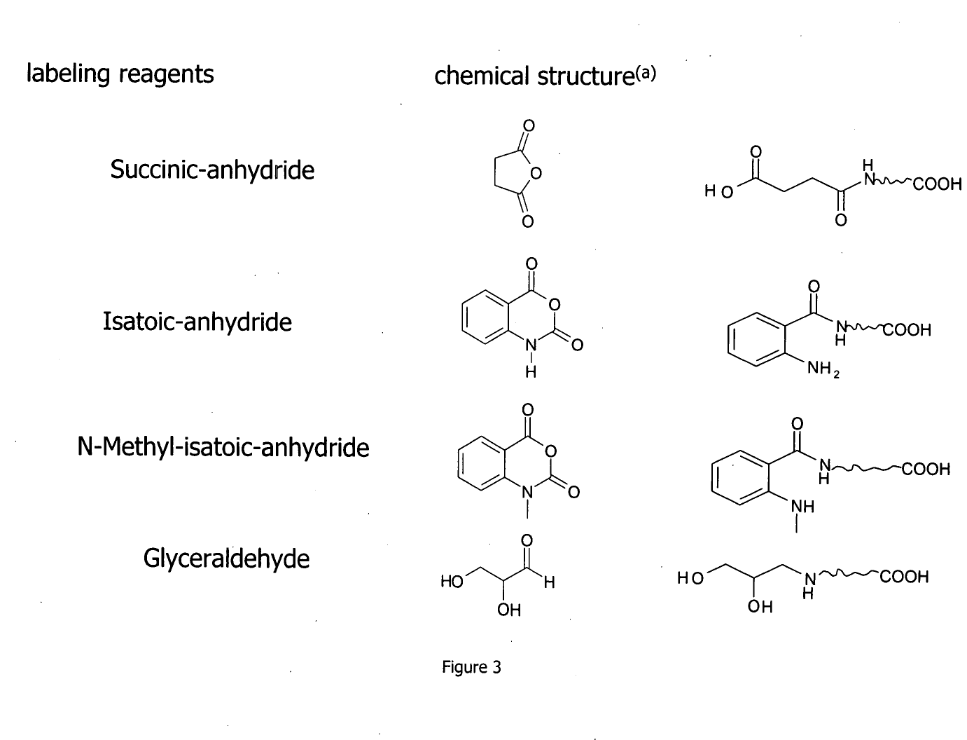

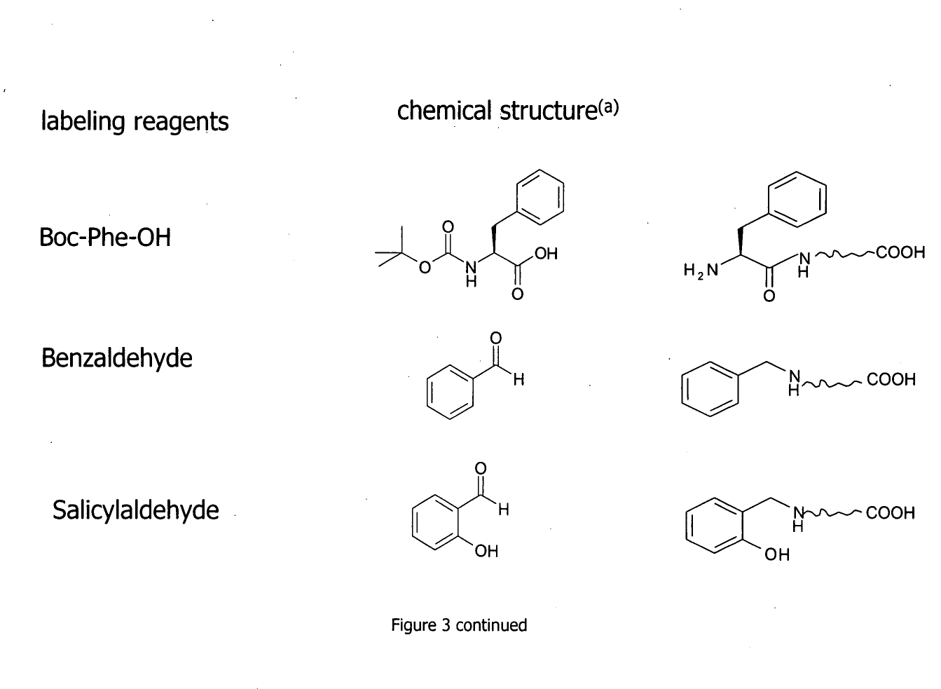

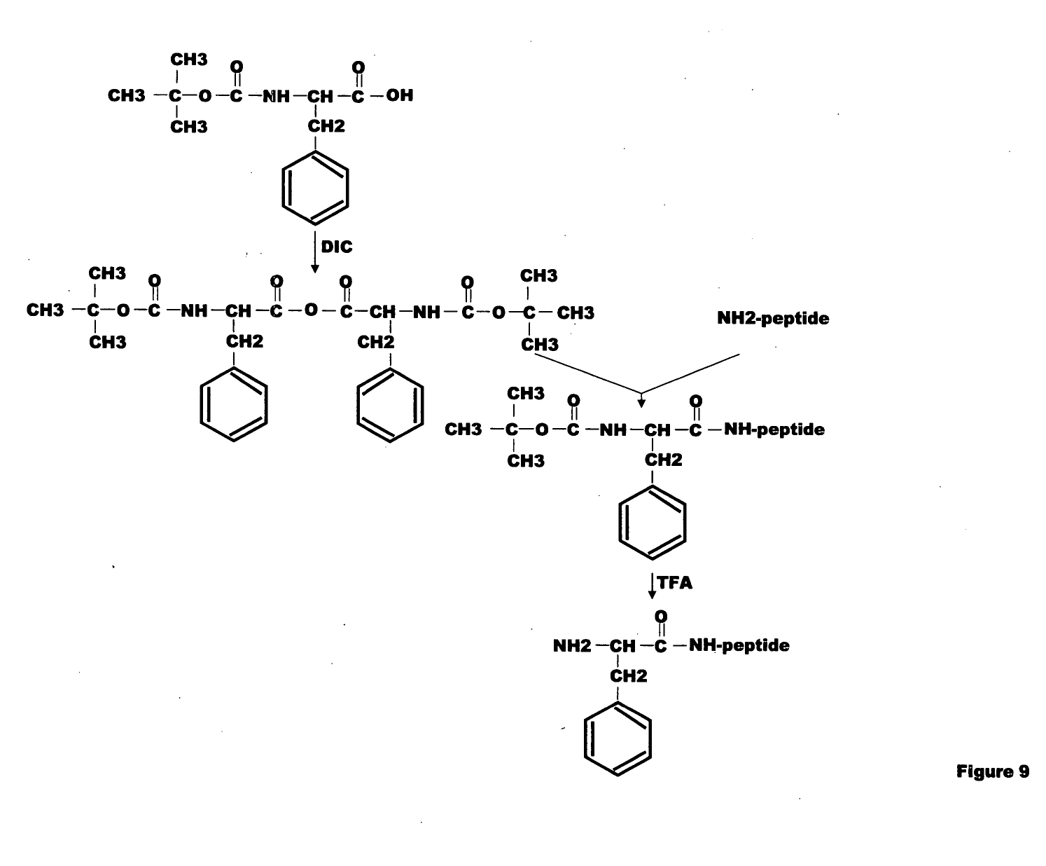

In contrast to these previously described isotope tags related to an ICAT-type reagent, it is not required that an affinity tag be included in the reagent since the glycopolypeptides are already isolated. One skilled in the art can readily determine any of a number of appropriate isotope tags useful in methods of the invention. An isotope tag can be an alkyl, akenyl, alkynyl, alkoxy, aryl, and the like, and can be optionally substituted, for example, with O, S, N, and the like, and can contain an amine, carboxyl, sulfhydryl, and the like (see WO 00/11208). Exemplary isotope tags include succinic anhydride, isatoic-anhydride, N-methyl-isatoic-anhydride, glyceraldehyde, Boc-Phe-OH, benzaldehyde, salicylaldehyde, and the like (FIG. 3). In addition to Phe, as shown in FIGS. 3 and 9, other amino acids similarly can be used as isotope tags. Furthermore, small organic aldehydes, similar to those shown in FIG. 3, can be used as isotope tags. These and other derivatives can be made in the same manner as that disclosed herein using methods well known to those skilled in the art. One skilled in the art will readily recognize that a number of suitable chemical groups can be used as an isotope tag so long as the isotope tag can be differentially isotopically labeled.

The bound glycopeptide fragments are tagged with an isotope tag to facilitate MS analysis. In order to tag the glycopeptide fragments, the isotope tag contains a reactive group that can react with a chemical group on the peptide portion of the glycopeptide fragments. A reactive group is reactive with and therefore can be covalently coupled to a molecule in a sample such as a polypeptide. Reactive groups are well known to those skilled in the art (see, for example, Hermanson, Bioconjugate Techniques, pp. 3-166, Academic Press, San Diego (1996); Glazer et al., Laboratory Techniques in Biochemistry and Molecular Biology: Chemical Modification of Proteins, Chapter 3, pp. 68-120, Elsevier Biomedical Press, New York (1975); Pierce Catalog (1994), Pierce, Rockford Ill.). Any of a variety of reactive groups can be incorporated into an isotope tag for use in methods of the invention so long as the reactive group can be covalently coupled to the immobilized polypeptide.

To analyze a large number or essentially all of the bound glycopolypeptides, it is desirable to use an isotope tag having a reactive group that will react with the majority of the glycopeptide fragments. For example, a reactive group that reacts with an amino group can react with the free amino group at the N-terminus of the bound glycopeptide fragments. If a cleavage reagent is chosen that leaves a free amino group of the cleaved peptides, such an amino group reactive agent can label a large fraction of the peptide fragments. Only those with a blocked N-terminus would not be labeled. Similarly, a cleavage reagent that leaves a free carboxyl group on the cleaved peptides can be modified with a carboxyl reactive group, resulting in the labeling of many if not all of the peptides. Thus, the inclusion of amino or carboxyl reactive groups in an isotope tag is particularly useful for methods of the invention in which most if not all of the bound glycopeptide fragments are desired to be analyzed.

In addition, a polypeptide can be tagged with an isotope tag via a sulfhydryl reactive group, which can react with free sulfhydryls of cysteine or reduced cystines in a polypeptide. An exemplary sulfhydryl reactive group includes an iodoacetamido group (see Gygi et al., supra, 1999). Other examplary sulfhydryl reactive groups include maleimides, alkyl and aryl halides, haloacetyls, α-haloacyls, pyridyl disulfides, aziridines, acrylolyls, arylating agents and thiomethylsulfones.

A reactive group can also react with amines such as the a-amino group of a peptide or the E-amino group of the side chain of Lys, for example, imidoesters, N-hydroxysuccinimidyl esters (NHS), isothiocyanates, isocyanates, acyl azides, sulfonyl chlorides, aldehydes, ketones, glyoxals, epoxides (oxiranes), carbonates, arylating agents, carbodiimides, anhydrides, and the like. A reactive group can also react with carboxyl groups found in Asp or Glu or the C-terminus of a peptide, for example, diazoalkanes, diazoacetyls, carbonyldiimidazole, carbodiimides, and the like. A reactive group that reacts with a hydroxyl group includes, for example, epoxides, oxiranes, carbonyldiimidazoles, N,N′-disuccinimidyl carbonates, N-hydroxycuccinimidyl chloroformates, and the like. A reactive group can also react with amino acids such as histidine, for example, a-haloacids and amides; tyrosine, for example, nitration and iodination; arginine, for example, butanedione, phenylglyoxal, and nitromalondialdehyde; methionine, for example, iodoacetic acid and iodoacetamide; and tryptophan, for example, 2-(2-nitrophenylsulfenyl)-3-methyl-3-bromoindolenine (BNPS-skatole), N-bromosuccinimide, formylation, and sulfenylation (Glazer et al., supra, 1975). In addition, a reactive group can also react with a phosphate group for selective labeling of phosphopeptides (Zhou et al., Nat. Biotechnol., 19:375-378 (2001)) or with other covalently modified peptides, including lipopeptides, or any of the known covalent polypeptide modifications. One skilled in the art can readily determine conditions for modifying sample molecules by using various reagents, incubation conditions and time of incubation to obtain conditions suitable for modification of a molecule with an isotope tag. The use of covalent-chemistry based isolation methods is particularly useful due to the highly specific nature of the binding of the glycopolypeptides.

The reactive groups described above can form a covalent bond with the target sample molecule. However, it is understood that an isotope tag can contain a reactive group that can non-covalently interact with a sample molecule so long as the interaction has high specificity and affinity.

Prior to further analysis, it is generally desirable to release the bound glycopeptide fragments. The glycopeptide fragments can be released by cleaving the fragments from the solid support, either enzymatically or chemically. For example, glycosidases such as N-glycosidases and O-glycosidases can be used to cleave an N-linked or O-linked carbohydrate moiety, respectively, and release the corresponding de-glycosylated peptide(s). If desired, N-glycosidases and O-glycosidases can be added together or sequentially, in either order. The sequential addition of an N-glycosidase and an O-glycosidase allows differential characterization of those released peptides that were N-linked versus those that were O-linked, providing additional information on the nature of the carbohydrate moiety and the modified amino acid residue. Thus, N-linked and O-linked glycosylation sites can be analyzed sequentially and separately on the same sample, increasing the information content of the experiment and simplifying the complexity of the samples being analyzed.

In addition to N-glycosidases and O-glycosidases, other glycosidases can be used to release a bound glycopolypeptide. For example, exoglycosidases can be used. Exoglycosidases are anomeric, residue and linkage specific for terminal monnosaccharides and can be used to release peptides having the corresponding carbohydrate.

In addition to enzymatic cleavage, chemical cleavage can also be used to cleave a carbohydrate moiety to release a bound peptide. For example, O-linked oligosaccharides can be released specifically from a polypeptide via a β-elimination reaction catalyzed by alkali. The reaction can be carried out in about 50 mM NaOH containing about 1 M NaBH₄ at about 55° C. for about 12 hours. The time, temperature and concentration of the reagents can be varied so long as a sufficient β-elimination reaction is carried out for the needs of the experiment.

In one embodiment, N-linked oligosaccharides can be released from glycopolypeptides, for example, by hydrazinolysis. Glycopolypeptides can be dried in a desiccator over P₂O₅ and NaOH. Anhydrous hydrazine is added and heated at about 100° C. for 10 hours, for example, using a dry heat block.

In addition to using enzymatic or chemical cleavage to release a bound glycopeptide, the solid support can be designed so that bound molecules can be released, regardless of the nature of the bound carbohydrate. The reactive group on the solid support, to which the glycopolypeptide binds, can be linked to the solid support with a cleavable linker. For example, the solid support reactive group can be covalently bound to the solid support via a cleavable linker such as a photocleavable linker. Exemplary photocleavable linkers include, for example, linkers containing o-nitrobenzyl, desyl, trans-o-cinnamoyl, m-nitrophenyl, benzylsulfonyl groups (see, for example, Dorman and Prestwich, Trends Biotech. 18:64-77 (2000); Greene and Wuts, Protective Groups in Organic Synthesis, 2nd ed., John Wiley & Sons, New York (1991); U.S. Pat. Nos. 5,143,854; 5,986,076; 5,917,016; 5,489,678; 5,405,783). Similarly, the reactive group can be linked to the solid support via a chemically cleavable linker. Release of glycopeptide fragments with the intact carbohydrate is particularly useful if the carbohydrate moiety is to be characterized using well known methods, including mass spectrometry. The use of glycosidases to release de-glycosylated peptide fragments also provides information on the nature of the carbohydrate moiety.

Thus, the invention provides methods for identifying a glycopolypeptide and, furthermore, identifying its glycosylation site. The methods of the invention are applied, as disclosed herein, and the parent glycopolypeptide is identified. The glycosylation site itself can also be identified and consensus motifs determined (Example VII), as well as the carbohydrate moiety, as disclosed herein. The invention further provides glycopolypeptides, glycopeptides and glycosylation sites identified by the methods of the invention.

Glycopolypeptides from a sample are bound to a solid support via the carbohydrate moiety. The bound glycopolypeptides are generally cleaved, for example, using a protease, to generate glycopeptide fragments. As discussed above, a variety of methods can be used to release the bound glycopeptide fragments, thereby generating released glycopeptide fragments. As used herein, a “released glycopeptide fragment” refers to a peptide which was bound to a solid support via a covalently bound carbohydrate moiety and subsequently released from the solid support, regardless of whether the released peptide retains the carbohydrate. In some cases, the method by which the bound glycopeptide fragments are released results in cleavage and removal of the carbohydrate moiety, for example, using glycosidases or chemical cleavage of the carbohydrate moiety. If the solid support is designed so that the reactive group, for example, hydrazide, is attached to the solid support via a cleavable linker, the released glycopeptide fragment retains the carbohydrate moiety. It is understood that, regardless whether a carbohydrate moiety is retained or removed from the released peptide, such peptides are referred to as released glycopeptide fragments.

After isolating glycopolypeptides from a sample and cleaving the glycopolypeptide into fragments, the glycopeptide fragments released from the solid support and the released glycopeptide fragments are identified and/or quantitified. A particularly useful method for analysis of the released glycopeptide fragments is mass spectrometry. A variety of mass spectrometry systems can be employed in the methods of the invention for identifying and/or quantifying a sample molecule such as a released glycopolypeptide fragment. Mass analyzers with high mass accuracy, high sensitivity and high resolution include, but are not limited to, ion trap, triple quadrupole, and time-of-flight, quadrupole time-of-flight mass spectrometeres and Fourier transform ion cyclotron mass analyzers (FT-ICR-MS). Mass spectrometers are typically equipped with matrix-assisted laser desorption (MALDI) and electrospray ionization (ESI) ion sources, although other methods of peptide ionization can also be used. In ion trap MS, analytes are ionized by ESI or MALDI and then put into an ion trap. Trapped ions can then be separately analyzed by MS upon selective release from the ion trap. Fragments can also be generated in the ion trap and analyzed. Sample molecules such as released glycopeptide fragments can be analyzed, for example, by single stage mass spectrometry with a MALDI-TOF or ESI-TOF system. Methods of mass spectrometry analysis are well known to those skilled in the art (see, for example, Yates, J. Mass Spect. 33:1-19 (1998); Kinter and Sherman, Protein Sequencing and Identification Using Tandem Mass Spectrometry, John Wiley & Sons, New York (2000); Aebersold and Goodlett, Chem. Rev. 101:269-295 (2001)).

For high resolution polypeptide fragment separation, liquid chromatography ESI-MS/MS or automated LC-MS/MS, which utilizes capillary reverse phase chromatography as the separation method, can be used (Yates et al., Methods Mol. Biol. 112:553-569 (1999)). Data dependent collision-induced dissociation (CID) with dynamic exclusion can also be used as the mass spectrometric method (Goodlett, et al., Anal. Chem. 72:1112-1118 (2000)).

Once a peptide is analyzed by MS/MS, the resulting CID spectrum can be compared to databases for the determination of the identity of the isolated glycopeptide. Methods for protein identification using single peptides has been described previously (Aebersold and Goodlett, Chem. Rev. 101:269-295 (2001); Yates, J. Mass Spec. 33:1-19 (1998)). In particular, it is possible that one or a few peptide fragments can be used to identify a parent polypeptide from which the fragments were derived if the peptides provide a unique signature for the parent polypeptide. Thus, identification of a single glycopeptide, alone or in combination with knowledge of the site of glycosylation, can be used to identify a parent glycopolypeptide from which the glycopeptide fragments were derived. Further information can be obtained by analyzing the nature of the attached tag and the presence of the consensus sequence motif for carbohydrate attachment. For example, if peptides are modified with an N-terminal tag, each released glycopeptide has the specific N-terminal tag, which can be recognized in the fragment ion series of the CID spectra. Furthermore, the presence of a known sequence motif that is found, for example, in N-linked carbohydrate-containing peptides, that is, the consensus sequence NXS/T, can be used as a constraint in database searching of N-glycosylated peptides.

In addition, the identity of the parent glycopolypeptide can be determined by analysis of various characteristics associated with the peptide, for example, its resolution on various chromatographic media or using various fractionation methods. These empirically determined characteristics can be compared to a database of characteristics that uniquely identify a parent polypeptide, which defines a peptide tag.

The use of a peptide tag and related database is used for identifying a polypeptide from a population of polypeptides by determining characteristics associated with a polypeptide, or a peptide fragment thereof, comparing the determined characteristics to a polypeptide identification index, and identifying one or more polypeptides in the polypeptide identification index having the same characteristics (see WO 02/052259). The methods are based on generating a polypeptide identification index, which is a database of characteristics associated with a polypeptide. The polypeptide identification index can be used for comparison of characteristics determined to be associated with a polypeptide from a sample for identification of the polypeptide. Furthermore, the methods can be applied not only to identify a polypeptide but also to quantitate the amount of specific proteins in the sample.

The methods for identifying a polypeptide are applicable to performing quantitative proteome analysis, or comparisons between polypeptide populations that involve both the identification and quantification of sample polypeptides. Such a quantitative analysis can be conveniently performed in two separate stages, if desired. As a first step, a reference polypeptide index is generated representative of the samples to be tested, for example, from a species, cell type or tissue type under investigation, such as a glycopolypeptide sample, as disclosed herein. The second step is the comparison of characteristics associated with an unknown polypeptide with the reference polypeptide index or indices previously generated.

A reference polypeptide index is a database of polypeptide identification codes representing the polypeptides of a particular sample, such as a cell, subcellular fraction, tissue, organ or organism. A polypeptide identification index can be generated that is representative of any number of polypeptides in a sample, including essentially all of the polypeptides potentially expressed in a sample. In methods of the invention directed to identifying glycopolypeptides, the polypeptide identification index is determined for a desired sample such as a serum sample. Once a polypeptide identification index has been generated, the index can be used repeatedly to identify one or more polypeptides in a sample, for example, a sample from an individual potentially having a disease. Thus, a set of characteristics can be determined for glycopeptides that can be correlated with a parent glycopolypeptide, including the amino acid sequence of the glycopeptide, and stored as an index, which can be referenced in a subsequent experiment on a sample treated in substantially the same manner as when the index was generated.

The incorporation of an isotope tag can be used to facilitate quantification of the sample glycopolypeptides. As disclosed previously, the incorporation of an isotope tag provides a method for quantifying the amount of a particular molecule in a sample (Gygi et al., supra, 1999; WO 00/11208). In using an isotope tag, differential isotopes can be incorporated, which can be used to compare a known amount of a standard labeled molecule having a differentially labeled isotope tag from that of a sample molecule, as described in more detail below (see Example XIII). Thus, a standard peptide having a differential isotope can be added at a known concentration and analyzed in the same MS analysis or similar conditions in a parallel MS analysis. A specific, calibrated standard can be added with known absolute amounts to determine an absolute quantity of the glycopolypeptide in the sample. In addition, the standards can be added so that relative quantitation is performed, as described below.

Alternatively, parallel glycosylated sample molecules can be labeled with a different isotopic label and compared side-by-side (see Gygi et al., supra, 1999). This is particularly useful for qualitative analysis or quantitative analysis relative to a control sample. For example, a glycosylated sample derived from a disease state can be compared to a glycosylated sample from a non-disease state by differentially labeling the two samples, as described previously (Gygi et al., supra, 1999). Such an approach allows detection of differential states of glycosylation, which is facilitated by the use of differential isotope tags for the two samples, and can thus be used to correlate differences in glycosylation as a diagnostic marker for a disease (see Examples VIII, IX, XI and XII).

The methods of the invention provide numerous advantages for the analysis of complex biological and clinical samples. From every glycoprotein present in a complex sample, only a few peptides will be isolated since only a few peptides of a glycoprotein are glycosylated. Therefore, by isolating glycopeptide fragments, the composition of the resulting peptide mixture is significantly simplified for mass spectrometric analysis. For example, every protein on average will produce dozens of tryptic peptides but only one to a few tryptic glycosylated peptides. For example, the number of glycopeptides is significantly lower than the number of tryptic peptides or Cys-containing peptides in the major plasma proteins (see Table 1). Thus, analysis of glycopolypeptides or glycopeptides reduces the complexity of complex biological samples, for example, serum.

TABLE 1
Five major plasma proteins represent more than 80% total protein

| | Number of Peptides | | |
|---|---|---|---|
| Protein | Tryptic | Cys | Glyco |
| Albumin (40 mg/ml) | 82 | 27 | 0 |
| α1-antitrypsin (3 mg/ml) | 39 | 2 | 3 |
| α2-macroglobulin | 125 | 24 | 8 |
| Transferrin | 79 | 37 | 2 |
| γ-Globulin | 15 | 4 | 2 |
| Total | 340 | 94 | 15 |

Another advantage of the methods of the invention is the use for analysis of body fluids as a clinical specimen, in particular serum. Five major plasma proteins represent more than 80% of the total protein in plasma, albumin, α1 antitrypsin, α2 macroglobulin, transferrin, and γ-globulins. Of these, albumin is the most abundant protein in blood serum and other body fluids, constituting about 50% of the total protein in plasma. However, albumin is essentially transparent to the methods of the invention due to the lack of N-glycosylation. For example, no tryptic N-glycosylated peptides from albumin were observed when the methods of the invention were applied and a N-glycosidase was used to release the N-linked glycopeptides. This is all the more significant because more than 50 different albumin species have been detected by 2D gel electrophoresis that collectively obscure a significant part of the gel pattern and the analysis of less abundant serum proteins having clinical significance. Therefore, the methods of the invention that allow analysis of glycosylated proteins compensate for the dominance of albumin in serum and allow the analysis of less abundant, glycosylated proteins present in serum. As disclosed herein, the methods of the invention allowed the identification of many more serum proteins compared to conventional methods (see Example II). The methods of the invention also allow the analysis of less abundant serum proteins. These low abundance serum proteins are potential diagnostic markers. Such markers can be readily determined by comparing disease samples with healthy samples, as disclosed herein (see Examples VIII, IX, XI and XII).

Additionally, the known sequence motif for N-glycosylation (N-X-S/T) serves as a powerful sequence database search contraint for the identification of the isolated peptides. This can be used to facilitate the identification of the polypeptide from which the glycopeptide fragment was derived since a smaller number of possible peptides will contain the glycosylation motif.

The methods of the invention are also advantageous because they allow fast throughput and simplicity. Accordingly, the methods can be readily adapted for high throughput analysis of samples, which can be particularly advantageous for the analysis of clinical samples. Furthermore, the methods of the invention can be automated to facilitate the processing of multiple samples (see Example XVI). As disclosed herein, a robotic workstation has been adapted for automated glycoprotein analysis (Example XVI).

In addition to the analysis of body fluids for the reasons described above, the methods of the invention are also advantageous for the analysis of proteins contained in the plasma membrane. The methods of the invention allow for the selective separation of cell surface proteins and secreted proteins based on the fact that the proteins most likely contaminating such specimens, intracellular proteins, are very unlikely to be glycosylated. Thus, the methods of the invention can be used to more accurately reflect proteins representative of the sample rather than contaminants from cell lysis. Such an analysis can be optionally combined with subcellular fractionation for the analysis of glycopolypeptides (Example IV).

As described above, non-glycosylated peptide fragments are released from the solid support after proteolytic or chemical cleavage (see FIG. 1). If desired, the released peptide fragments can be characterized to provide further information on the nature of the glycopolypeptides isolated from the sample. A particularly useful method is the use of the isotope-coded affinity tag (ICAT™) method (Gygi et al., Nature Biotechnol. 17:994-999 (1999) which is incorporated herein by reference). The ICAT™ type reagent method uses an affinity tag that can be differentially labeled with an isotope that is readily distinguished using mass spectrometry. The ICAT™ type affinity reagent consists of three elements, an affinity tag, a linker and a reactive group.

One element of the ICAT™ type affinity reagent is an affinity tag that allows isolation of peptides coupled to the affinity reagent by binding to a cognate binding partner of the affinity tag. A particularly useful affinity tag is biotin, which binds with high affinity to its cognate binding partner avidin, or related molecules such as streptavidin, and is therefore stable to further biochemical manipulations. Any affinity tag can be used so long as it provides sufficient binding affinity to its cognate binding partner to allow isolation of peptides coupled to the ICAT™ type affinity reagent. An affinity tag can also be used to isolate a tagged peptide with magnetic beads or other magnetic format suitable to isolate a magnetic affinity tag. In the ICAT™ type reagent method, or any other method of affinity tagging a peptide, the use of covalent trapping, for example, using a cross-linking reagent, can be used to bind the tagged peptides to a solid support, if desired.

A second element of the ICAT™ type affinity reagent is a linker that can incorporate a stable isotope. The linker has a sufficient length to allow the reactive group to bind to a specimen polypeptide and the affinity tag to bind to its cognate binding partner. The linker also has an appropriate composition to allow incorporation of a stable isotope at one or more atoms. A particularly useful stable isotope pair is hydrogen and deuterium, which can be readily distinguished using mass spectrometry as light and heavy forms, respectively. Any of a number of isotopic atoms can be incorporated into the linker so long as the heavy and light forms can be distinguished using mass spectrometry. Exemplary linkers include the 4,7,10-trioxa-1,13-tridecanediamine based linker and its related deuterated form, 2,2′,3,3′,11,11′,12,12′-octadeutero-4,7,10-trioxa-1,13-tridecanediamine, described by Gygi et al. (supra, 1999). One skilled in the art can readily determine any of a number of appropriate linkers useful in an ICAT™ type affinity reagent that satisfy the above-described criteria, as described above for the isotope tag.

The third element of the ICAT™ type affinity reagent is a reactive group, which can be covalently coupled to a polypeptide in a specimen. Various reactive groups have been described above with respect to the isotope tag and can similarly be incorporated into an ICAT-type reagent.

The ICAT™ method or other similar methods can be applied to the analysis of the non-glycosylated peptide fragments released from the solid support. Alternatively, the ICAT™ method or other similar methods can be applied prior to cleavage of the bound glycopolypeptides, that is, while the intact glycopolypeptide is still bound to the solid support.

The method generally involves the steps of automated tandem mass spectrometry and sequence database searching for peptide/protein identification; stable isotope tagging for quantification by mass spectrometry based on stable isotope dilution theory; and the use of specific chemical reactions for the selective isolation of specific peptides. For example, the previously described ICAT™ reagent contained a sulfhydryl reactive group, and therefore an ICAT™-type reagent can be used to label cysteine-containing peptide fragments released from the solid support. Other reactive groups, as described above, can also be used.

The analysis of the non-glycosylated peptides, in conjunction with the methods of analyzing glycosylated peptides, provides additional information on the state of polypeptide expression in the sample. By analyzing both the glycopeptide fragments as well as the non-glycosylated peptides, changes in glycoprotein abundance as well as changes in the state of glycosylation at a particular glycosylation site can be readily determined.

If desired, the sample can be fractionated by a number of known fractionation techniques. Fractionation techniques can be applied at any of a number of suitable points in the methods of the invention. For example, a sample can be fractionated prior to oxidation and/or binding of glycopolypeptides to a solid support. Thus, if desired, a substantially purified fraction of glycopolypeptide(s) can be used for immobilization of sample glycopolypeptides. Furthermore, fractionation/purification steps can be applied to non-glycosylated peptides or glycopeptides after release from the solid support. One skilled in the art can readily determine appropriate steps for fractionating sample molecules based on the needs of the particular application of methods of the invention.

Methods for fractionating sample molecules are well known to those skilled in the art. Fractionation methods include but are not limited to subcellular fractionation or chromatographic techniques such as ion exchange, including strong and weak anion and cation exchange resins, hydrophobic and reverse phase, size exclusion, affinity, hydrophobic charge-induction chromatography, dye-binding, and the like (Ausubel et al., Current Protocols in Molecular Biology (Supplement 56), John Wiley & Sons, New York (2001); Scopes, Protein Purification: Principles and Practice, third edition, Springer-Verlag, New York (1993)). Other fractionation methods include, for example, centrifugation, electrophoresis, the use of salts, and the like (see Scopes, supra, 1993). In the case of analyzing membrane glycoproteins, well known solubilization conditions can be applied to extract membrane bound proteins, for example, the use of denaturing and/or non-denaturing detergents (Scopes, supra, 1993).

Affinity chromatography can also be used including, for example, dye-binding resins such as Cibacron blue, substrate analogs, including analogs of cofactors such as ATP, NAD, and the like, ligands, specific antibodies useful for immuno-affinity isolation, either polyclonal or monoclonal, and the like. A subset of glycopolypeptides can be isolated using lectin affinity chromatography, if desired. An exemplary affinity resin includes affinity resins that bind to specific moieties that can be incorporated into a polypeptide such as an avidin resin that binds to a biotin tag on a sample molecule labeled with an ICAT™-type reagent. The resolution and capacity of particular chromatographic media are known in the art and can be determined by those skilled in the art. The usefulness of a particular chromatographic separation for a particular application can similarly be assessed by those skilled in the art.

Those of skill in the art will be able to determine the appropriate chromatography conditions for a particular sample size or composition and will know how to obtain reproducible results for chromatographic separations under defined buffer, column dimension, and flow rate conditions. The fractionation methods can optionally include the use of an internal standard for assessing the reproducibility of a particular chromatographic application or other fractionation method. Appropriate internal standards will vary depending on the chromatographic medium or the fractionation method used. Those skilled in the art will be able to determine an internal standard applicable to a method of fractionation such as chromatography. Furthermore, electrophoresis, including gel electrophoresis or capillary electrophoresis, can also be used to fractionate sample molecules.

The invention also provides a method for identifying and quantifying glycopeptides in a sample. The method includes the steps of immobilizing glycopolypeptides to a solid support; cleaving the immobilized glycopolypeptides, thereby releasing non-glycosylated peptides and retaining immobilized glycopeptides; labeling the immobilized glycopeptides with an isotope tag; releasing the glycopeptides from the solid support; and analyzing the released glycopeptides.

The methods of the invention can be used in a wide range of applications in basic and clinical biology. The methods of the invention can be used for the detection of changes in the profile of proteins expressed in the plasma membrane, changes in the composition of proteins secreted by cells and tissues, changes in the protein composition of body fluids including blood and seminal plasma, cerebrospinal fluid, pancreatic juice, urine, breast milk, lung lavage, and the like. Since many of the proteins in these samples are glycosylated, the methods of the invention allow the convenient analysis of glycoproteins in these samples. Detected changes observed in a disease state can be used as diagnostic or prognostic markers for a wide range of diseases, including congenital disorders of glycosylation (Example XI) or any disorder involving aberrant glycosylation; cancer, such as skin, prostate, breast, colon, lung, and others (Examples VIII and IX); metabolic diseases or processes such as diabetes (Example XII) or changes in physiological state (Example X); inflammatory diseases such as rheumatoid arthritis; mental disorders or neurological processes; infectious disease; immune response to pathogens; and the like. Furthermore, the methods of the invention can be used for the identification of potential targets for a variety of therapies including antibody-dependent cell cytotoxicity directed against cell surface proteins and for detection of proteins accessible to drugs.

Thus, the methods of the invention can be used to identify diagnostic markers for a disease by comparing a sample from a patient having a disease to a sample from a healthy individual or group of individuals. By comparing disease and healthy samples, a diagnostic pattern can be determined with increases or decreases in expression of particular glycopolypeptides correlated with the disease, which can be used for subsequent analysis of samples for diagnostic purposes (see Examples VIII, IX, XI and XII). The methods are based on analysis of glycopolypeptides, and such an analysis is sufficient for diagnostic purposes.

Thus, the invention provides a method for identifying diagnostic glycopolypeptide markers by using a method of the invention and comparing samples from diseased individual(s) to healthy individual(s) and identifying glycopolypeptides having differential expression between the two samples, whereby differences in expression indicates a correlation with the disease and thus can function as a diagnostic marker. The invention also provides the diagnostic markers identified using methods of the invention.

Furthermore, glycopolypeptides exhibiting differential expression are potential therapeutic targets. Because they are differentially expressed, modulating the activity of these glycopolypeptides can potentially be used to ameliorate a sign or symptom associated with the disease. Thus, the invention provides a method for identifying therapeutic glycopolypeptide targets of a disease. Once a glycopolypeptide is found to be differentially expressed, the potential target can be screened for potential therapeutic agents that modulate the activity of the therapeutic glycopolypeptide target. Methods of generating libraries and screening the libraries for potential therapeutic activity are well known to those skilled in the art. Methods for producing pluralities of compounds, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art (see, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czamik, eds., Combinatorial Chemistry: Svnthesis and Application, John Wiley & Sons, New York (1997)). The invention additionally provides glycopolypeptide therapeutic targets identified by methods of the invention.

The methods can be used for a variety of clinical and diagnostic applications. Known therapeutic methods effected through glycopolypeptides can be characterized by methods of the invention. For example, therapies such as Enbrel™ and Herceptin function through glycoproteins. The methods of the invention allow characterization of individual patients with respect to glycoprotein expression, which can be used to determine likely efficacy of therapy involving glycoproteins.

Thus, the methods of the invention can be used in a variety of applications including, but not limited to, the following applications. The methods of the invention can be used, for example, for blood serum profiling for the detection of prognostic and diagnostic protein markers (see Examples VIII, IX, XI and XII). The methods of the invention can also be used for quantitative profiling of cell surface proteins for the detection of diagnostic/prognostic protein markers and the detection of potential targets of therapy (Example IV). For example, the methods of the invention can be used for antibody-dependent cellular cytotoxicity (ADCC) or other types of therapy. The methods of the invention are applicable in clinical and diagnostic medicine, veterinary medicine, agriculture, and the like. For example, the methods of the invention can be used to identify and/or validate drug targets and to evaluate drug efficacy, drug dosing, and/or drug toxicity. In such a case, the blood proteome, that is serum, can be analyzed using the methods disclosed herein to look for changes in serum glycopolypeptide profiles associated with drug administration and correlated with the effects of drug efficacy, dosing and/or toxicity, and/or validation of drug targets. Such a correlation can be readily determined by collecting serum samples from one or more individuals adminstered various drug doses, experiencing drug toxicity, experiencing a desired efficacy, and the like. In addition, a serum profile can be generated in combination with the analysis of drug targets as a way to rapidly and efficiently validate a particular target with the administration of a drug or various drug doses, toxicity, and the like. Thus, serum (blood samples) provide a surrogate marker for the status of an individual and his or her ability to respond to a pharmacological intervention.

The methods of the invention can additionally be used for quantitative protein profiling in various body fluids in addition to blood plasma, including CSF, pancreatic juice, lung lavage fluid, seminal plasma, urine, breast milk, and the like. The methods of the invention can also be used for quantitative protein profiling of proteins secreted by cells or tissues for the detection of new protein and peptide hormones and other factors. Thus, the invention provides a method to generate quantitative profiles of glycoproteins. The invention also provides a method for quantifying a glycopolypeptide in a sample, as disclosed herein. The invention further provides a method for the detection of prognostic or diagnostic patterns in blood serum and other body fluids. The invention additionally provides a method for the detection of secreted protein hormones and regulatory factors. Thus, the invention provides a method for profiling glycopolypeptides from body fluids, secreted proteins and cell surface proteins.

The methods of the invention are also applicable to the detection of changes in the state of glycosylation of proteins based on the concurrent application of protein abundance measurement and measurement of protein glycosylation on the same sample. Thus, the invention provides a method to detect quantitative changes in the glycosylation pattern of specific proteins.

The invention also provides a method for the systematic detection of glycosylation sites on proteins. Because the methods of the invention allow the identification of peptide fragments that are glycosylated, this also serves as the identification of the site of glycosylation (Example VII).

Although the methods disclosed herein have generally been described for the analysis of glycopolypeptides, similar methods are also applicable to the analysis of other carbohydrate-containing molecules. Because the methods are based on the specific binding of carbohydrate moieties, the methods of modification and/or isolation can similarly be applied to other carbohydrate-containing molecules. For example, method steps analogous to those disclosed herein can be applied to the identification and quantification of glycosylated molecules such as glycolipids, glycosphingolipids, and the like.

The invention also provides reagents and kits for isolating and quantifying glycopolypeptides. The kit can contain, for example, hydrazide resin or other suitably reactive resin for solid phase capture of glycopolypeptides, a reagent for modification of carbohydrate moieties, for example, an oxidizing reagent such as periodate, and a set of two or more differentially labeled isotope tags for coupling to two different samples, which are particularly useful for quantitative analysis using mass spectrometry. In one embodiment, the invention provides a kit comprising a hydrazide resin, periodate, and a pair of differentially labeled isotope tags. The contents of the kit of the invention, for example, any resins or labeling reagents, are contained in suitable packaging material, and, if desired, a sterile, contaminant-free environment. In addition, the packaging material contains instructions indicating how the materials within the kit can be employed to label sample molecules. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

The methods of the invention can be facilitated by the use of combinations of hardware and software suitable for analysis of methods of the invention. For example, a robotics workstation was developed to facilitate automated glycopeptide analysis (Example XVI). A computer program can be used to find patterns of proteins and/or peptides that are specifically present or present at specific abundances in a sample from a person with a specific disease (see Examples). For example, a number of serum samples can be analyzed and compared to serum samples from healthy individuals. An algorithm is used to find those peptides and/or proteins that are either individually or collectively diagnostic for the disease or the stage of the disease being examined.

In another embodiment, the invention provides a method for identifying and quantifying glycopeptides in a sample. The method can include the steps of immobilizing glycopolypeptides to a solid support; cleaving the immobilized glycopolypeptides, thereby releasing non-glycosylated peptides and retaining immobilized glycopeptides; releasing the glycopeptides from the solid support; and analyzing the released glycopeptides. The method can further include the step of identifying one or more glycopeptides, for example, using mass spectrometry.

In still another embodiment, the invention provides a method of identifying a diagnostic marker for a disease. The method can include the steps of immobilizing glycopolypeptides from a test sample to a first solid support; immobilizing glycopolypeptides from a control sample to a second solid support; cleaving the immobilized glycopolypeptides, thereby releasing non-glycosylated peptides and retaining immobilized glycopeptides; labeling the immobilized glycopeptides on the first and second supports with differential isotope tags on the respective supports; releasing the glycopeptides from the solid supports; analyzing the released glycopeptides; and identifying one or more glycosylated polypeptides having differential glycosylation between the test sample and the control sample. Alternatively, the test and control samples can be run in parallel and analyzed separately. In such a case, the glycopeptides are identified and compared without using differential isotope tagging.

The test sample can be, for example, a specimen from an individual having a disease. The control sample can be, for example, a corresponding specimen obtained from a healthy individual. The sample can be, for example, serum or a tissue biopsy, as described herein. Differential glycosylation can be a qualitative difference, for example, the presence or absence of a glycopolypeptide in the test sample compared to the control sample. Differential glycosylation can also be a quantitative difference. The determination of quantitative differences can be facilitated by the labeling with differential isotope tags such that the samples can be mixed and compared side-by-side, as disclosed herein and described in Gygi et al., supra, 1999. One or more glycopolypeptides exhibiting differential glycosylation are potential diagnostic markers for the respective disease. Such a method provides a glycopolypeptide disease profile, which can be used subsequently for diagnostic purposes. Accordingly, rather than using one or a few diagnostic markers, the methods of the invention allow the identification of a profile of diagnostic markers, which can provide more detailed information on the type of disease, the stage of disease, and/or the prognosis of a disease by determining profiles correlated with the type, stage and/or prognosis of a disease.

In yet another embodiment, the invention provides a method of diagnosing a disease. The method can include the steps of immobilizing glycopolypeptides from a test sample to a solid support; cleaving the immobilized glycopolypeptides, thereby releasing non-glycosylated peptides and retaining immobilized glycopeptides; releasing the glycopeptides from the solid support; analyzing the released glycopeptides; and identifying one or more diagnostic markers associated with a disease, for example, as determined by methods of the invention, as described above.

A test sample from an individual to be tested for a disease or suspected of having a disease can be processed as described for glycopeptide analysis by the methods disclosed herein. The resulting glycopeptide profile from the test sample can be compared to a control sample to determine if changes in glycosylation of diagnostic markers has occurred, as discussed above. Alternatively, the glycopeptide profile can be compared to a known set of diagnostic markers or a database containing information on diagnostic markers.

In another embodiment, the method of diagnosing a disease can include the step of generating a report on the results of the diagnostic test. For example, the report can indicate whether an individual is likely to have a disease or is likely to be disease free based on the presence of a sufficient number of diagnostic markers associated with a disease. The invention further provides a report of the outcome of a method of diagnosing a disease. Similar reports and preparation of such reports are provided for other methods of the invention.

It is understood that the methods of the invention can be performed in any order suitable for glycopolypeptide analysis. One skilled in the art can readily determine an appropriate order of carrying out steps of methods of the invention suitable for glycopeptide analysis.

In another embodiment, the invention provides a method for identifying glycopolypeptides in a sample by first cleaving the glycopolypeptides into glycopeptide fragments before capturing glosylated peptides. Glycosylation is one of the most important and abundant post-translational modifications in nature (Parodi, Annu. Rev. Biochem. 69:69-93 (2000)). Glycoproteins play important roles during molecular and cellular recognition in development, growth, and cellular communication; and, in particular, are involved in cancer progression and immune responses (Helenius, Science 291:2364-2369 (2001); Lowe, Cell 104:809-812 (2001)). Glycoproteins have been used as therapeutic targets and biomarkers for cancer prognosis, diagnosis, and monitoring. Examples include the carcinoembryonic antigen in colon, breast, pancreatic, and lung cancers; Her2/neu in breast cancer; β human chorionic gonadotropin and α-fetoprotein in germ cell tumors; prostate-specific antigen in prostate cancer; and CA-125 in ovarian cancer (Diamandis, Mol. Cell. Proteomics 3:367-378 (2004); Zhang et al., Nat. Biotechnol. 21:660-666 (2003)). As systems biology begins to revolutionize the understanding of biology and biomedical sciences (Hood, Mech. Ageing Dev. 124:9-16 (2003); Hood et al., Science 306:640-643 (2004)), the ability to efficiently and comprehensively profile glycoproteins in biological samples of interest (such as cell extracts and body fluids) is critical to many biological and clinical researchers.

Tandem mass spectrometry with its superior sensitivity, accuracy, and throughput in protein and peptide identification is currently the most sophisticated and powerful tool for global proteomic studies including glycoproteome analysis. Because the enormous dynamic range of protein concentrations in biological samples is far beyond the analysis range of most techniques (10⁶ in mammalian cells and 10¹⁰ in blood), low-abundant proteins are masked by dominant proteins in global proteomics analysis (Aebersold and Cravatt, Trends Biotechnol. 20, S1-2 (2002); Hood, Mech. Ageing Dev. 124:9-16 (2003)). Indeed, just 22 proteins constitute about 99% of the blood protein mass—albumin alone is more than 50% of the mass. Front-end enrichment and fractionation methods prior to MS analysis are necessary to enhance the detection sensitivity to low-abundant proteins, a category that holds promising diagnostic and biological information (Anderson et al., Mol. Cell. Proteomics 1:845-867 (2002)). An effective enrichment of glycosylated proteins is important to decrease sample complexity and helps to unfold the glycoproteome comprehensively (Novotny and Mechref, J. Sep. Sci. 28:1956-1968 (2005)). Two strategies have emerged to enrich glycoproteins and/or glycopeptides: one is the “top down” strategy, in which glycoproteins are enriched at the protein level and then digested into peptides, for example, the lectin affinity capture (O'Shannessy and Quarles, J. Immunol. Methods 99:153-161 (1987)) and glycoprotein chemical capture (Zhang et al., supra, 2003) approaches; the other is the “bottom up” strategy, in which glycoproteins are digested first into peptides and then enriched directly, for example, glycopeptide enrichment by chromatography (Alvarez-Manilla et al., J. Proteome Res. 5, 701-708(2006); An et al., Anal. Chem. 75:5628-5637 (2003); Hagglund et al., J. Proteome Res. 3:556-566 (2004); Larsen et al., Mol. Cell. Proteomics 4:107-119 (2005); Wada et al., Anal. Chem. 76:6560-6565 (2004)).

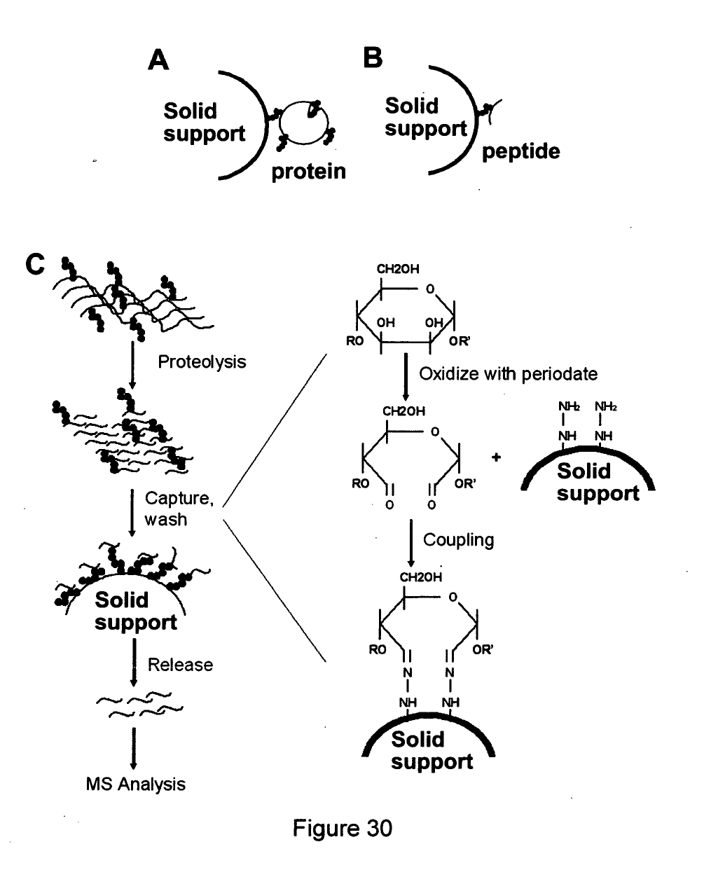

Despite the versatility of current glyco-enrichment approaches, for complex biological samples such as sera and cell lysates, it is cumbersome to unravel glycoproteome completely. For instance, the top-down strategy suffers from solubility problems and steric hindrance when capturing proteins in their native forms. Moreover, proteolysis of complex protein mixtures with trypsin, a commonly used proteolytic enzyme for tandem MS analysis, typically produces 20 or more peptides per protein, which results in increased sample complexity and is thereof not suitable for the analysis of low-abundance proteins in complex samples. Further enrichment of glycosylated peptides after glycoprotein capture has been studied both by lectin affinity capture (Kaji et al., Nat. Biotechnol. 21:667-672 (2003)) and glycoprotein chemical capture (Bobbitt et al., Adv. Carbohydr. Chem. 48:1-41 (1956)) approaches. Even though lectin affinity capture is the most widely used approach due to its ease of implementation, the binding selectivity of lectins to specific conformations of different carbohydrate moieties has limited the utility of lectins in global glycoprotein analysis (Lis et al., Chem. Rev. 98:637-674 (1998); Nilsson, Anal. Chem. 75:348A-353A (2003)). The glycoprotein-chemical-capture approach developed by Zhang et al. is generally applicable to all types of glycoproteins, but the complicated steps to implementation (Alvarez-Manilla, J. Proteome Res. 5:701-708 (2006)) and the relative lower yields (depicted in FIGS. 30A and 30B) lead to this approach not being used as widely as the lectin capture approach. In the bottom-up strategy, proteins are digested into peptides, and glycosylated peptides are separated from their unglycosylated counterparts by chromatography (Alvarez-Manilla et al., J. Proteome Res. 5:701-708 (2006); An et al., Anal. Chem. 75:5628-5637 (2003); Hagglund et al., J. Proteome Res. 3:556-566 (2004); Larsen et al., Mol. Cell. Proteomics 4:107-119 (2005); Wada et al., Anal. Chem. 76:6560-6565 (2004)). Although this approach is direct, simple and rapid; the separation based on different physical and chemical properties usually results in only a modest enrichment (Alvarez-Manilla et al., supra, 2006; Hagglund et al., supra, 2004).

As disclosed herein, a chemical-capture approach that focuses on a very efficient glycopeptide enrichment has been developed (see Example XVII). The approach provides optimized and robust selectivity for glycosylated peptides, improved identification of glycosylated membrane proteins, and enhanced MS detection sensitivity and accuracy to low-abundant but multi-glycosylated proteins. The strategy is illustrated in FIG. 30C. The feasibility was demonstrated and the capture efficiency of this approach was characterized using chicken avidin, a mono-glycosylated protein, and on a protein mixture consisting of five different glycoproteins containing up to 13 glycosylation sites. Close to 100% capture efficiency was obtained both for chicken avidin (using MALDI-TOF/TOF) and for the five-glycoprotein mixture (using LTQ LC-MS/MS). The capture approach was also applied to a complex and challenging biological mixture, the microsomal fractions from an ovarian cancer cell line IGROV-1/CP (a cisplatin-resistant ovarian-cancer cell line derived from the cisplatin-sensitive ovarian-cancer cell line, IGROV-1). During a typical nanoLC-MS analysis, a total of 156 unique proteins and 311 unique peptides were identified, which includes 68 proteins with multiple peptide hits. The glycopeptide specificity of the approach is 91%. From four LTQ experiments of two biological replicates (two repeated LTQ runs per each biological replicate), a total of 302 proteins were identified with an average protein identification rate of 136±19 per LTQ MS run (n=4) and a selectivity of (91±1.6) % for the N-linked glyco-consensus sequence (n=4). A value of 0.9 was used as a cutoff of the protein-prophet probability value in all of the analyses, and on average the error rate of the analysis was as small as 0.006 in all four MS runs, and the averaged number of incorrectly identified peptides was 1 out of 136 by statistical analysis (Nesvizhskii et al., Anal. Chem. 75:4646-4658 (2003)).

The method described herein of capturing glycoprotein at the peptide rather than the protein level permits the maximum capture possibility, with which the downstream analysis such as mass spectrometry based identification and quantification can be dramatically improved. There are several reasons for this improvement. First, it was shown that about one third glycans in N-glycosylation site fill a groove or hole of the topography of a protein that makes extensive contacts between the glycans and the protein surface. Such structure increases the steric hindrance for glycan being oxidized and captured by external reagents. Digesting proteins into peptides can exposure all the glyans and render them accessible to capture (Petrescu et al., Glycobiology 14:103-114 (2004)). Second, a large percentage of interesting glycoproteins are membrane proteins that are hard to dissolve in aqueous solutions and therefore are difficult to capture at the protein level. Rupturing the hierarchy structure of proteins by denaturing buffer and digesting proteins into peptides can effectively improve the extrapolating efficiency of protein into solution and the chances for them to be analyzed later. Third, because of the exclusive accuracy, sensitivity, and high throughput, mass spectrometry is widely used for proteomic analysis, including glyco proteomic approaches. Since mass spectrometry analyzes proteins at the peptide level, the glycopeptide approach is well suited for mass spectrometry based analysis and simplified the sample preparation procedure. One of the shortcomings of a multi-step chemical capture approach is the loss of analyzing compounds during processing procedures. To decrease sample loss and improve the capture efficiency, chemical quenching reactions can be applied to ensure that multiple reactions can be sequentially introduced into a single mixture without extra separation steps.

The glycopeptide capture technique disclosed herein is well suited to facilitate glycoprotein research. It is a proteomic technique that can capture and enrich a larger number, and potentially all, glycosylated peptides and allow annotation to the corresponding proteins in biological samples. The glycopeptide capture technique can globally disclose the glyco-constituents in a sample and can be easily coupled with quantification approaches for functional interpretation. Such a technique can be used to study glycoproteins that are involved in important biological processes as well as in prevention, prognosis, and treatment of diseases.

As disclosed herein, a robust and general shotgun glycoproteomics approach can be used to comprehensively profile glycoproteins in complex biological mixtures (see Example XVII). In this approach, glycopeptides derived from glycoproteins are enriched by selective capture onto a solid support using hydrazide chemistry, followed by enzymatic release of the peptides and subsequent analysis by tandem mass spectrometry. The approach was validated using standard protein mixtures which resulted in highly efficient capture efficiency. The capture approach was then applied to microsomal fractions of the cisplatin-resistant ovarian-cancer cell line IGROV-1/CP. With a protein-prophet probability value greater than 0.9, a total of 302 proteins were identified with an average protein identification rate of 136±19 (n=4) in a single LTQ nanoLC-MS experiment, and a selectivity of 91±1.6% (n=4) for the N-linked glyco-consensus sequence.

The methods dislcosed herein have several advantages. First, the utility of sodium-sulphite as a quencher in the capture approach to replace the solid phase extraction step in earlier glycoprotein chemical-capture approach for removing excess sodium periodate allows the overall capture procedure to be completed in a single vessel. This improvement minimizes sample loss and increases sensitivity, and makes the protocol amenable for high throughput implementation, a feature that is particularly useful for biomarker identification and validation of large number of clinical samples. Second, digestion of proteins initially into peptides improves solubility of large membrane proteins and exposes all of the glycosylation sites to ensure equal accessibility to capture reagents. Third, capturing glycosylated peptides can effectively reduce sample complexity and at the same time increase the confidence of MS-based protein identifications, that is, more potential peptide identifications per protein. Fourth, the approach is demonstrated herein on the analysis of N-linked glycopeptides, however, it can be applied equally well to O-glycoprotein analysis, as described herein.

In one embodiment, the invention provides a method for identifying glycopolypeptides in a sample. The method can include the steps of cleaving glycopolypeptides to generate glycopeptide fragments; derivatizing the glycopeptide fragments in a polypeptide sample; immobilizing the derivatized glycopeptide fragments to a solid support; releasing the glycopeptide fragments from the solid support, thereby generating released glycopeptide fragments; analyzing the released glycopeptide fragments using mass spectrometry; and identifying a released glycopeptide fragment. The method can further include labeling the immobilized glycopeptide fragments with an isotope tag. In addition, the method can further include quantifying the amount of the identified glycopeptide fragment.

In a particular emodiment, the solid support can comprise a hydrazide moiety. In another embodiment, the glycopeptide fragments are released from the solid support using a glycosidase, for example, N-glycosidase or an O-glycosidase, using either simultaneous or sequential addition of N-glycosidase and O-glycosidase. Alternatively, the glycopeptide fragments can be released from the solid support using chemical cleavage. The glycopeptide fragments can be oxidized with periodate. Furthermore, the glycopolypeptides can be cleaved with a protease such as trypsin to generate glycopeptide fragments. Exemplary samples from which the glycopolypeptide are obtained include a body fluid, secreted proteins, and cell surface proteins.

In still another embodiment, the invention provides a method for identifying glycopeptides in a sample. The method can include the steps of cleaving glycopolypeptides to generate glycopeptide fragments; immobilizing the glycopeptide fragments to a solid support; releasing the glycopeptide fragments from the solid support; and analyzing the released glycopeptide fragments. The method can further comprise labeling the immobilized glycpeptide fragments with an isotope tag. The glycopeptide fragments can be oxidized, for example, with periodate. The solid support in such a method can comprise a hydrazide moiety.

In a method of identifying glycopeptides in a sample, the glycopeptide fragments can be released from the solid support using a glycosidase, for example, an N-glycosidase or an O-glycosidase, added simultaneously or sequentially. Alternatively, the glycopeptide fragments can be released from the solid support using chemical cleavage. The glycopolypeptides can be cleaved with a protease such as trypsin to generate glycopeptide fragments.

In yet another embodiment, the invention provides a method of identifying a diagnostic marker for a disease. The method can include the steps of cleaving glycopolypeptides from a test sample to generate test glycopeptide fragments; cleaving glycopolypeptides from a control sample to generate control glycopeptide fragments; immobilizing the test glycopeptide fragments to a first solid support; immobilizing the control glycopeptide fragments from a control sample to a second solid support; releasing the test glycopeptide fragments and control glycopeptide fragments from the solid supports; analyzing the released glycopeptide fragments; and identifying one or more glycosylated polypeptides having differential glycosylation between the test sample and the control sample. Such a method can further comprise labeling the immobilized glycopeptide fragments on the first and second supports with differential isotope tags on the respective supports.

In a method of identifying a diagnostic marker for a disease, glycopeptide fragments can be oxidized, for example, with periodate. In addition, the solid support can comprise a hydrazide moiety. The glycopeptide fragments can be released from the solid support using a glycosidase, an N-glycosidase or an O-glycosidase, added simultaneously or sequentially. Alternatively, the glycopeptide fragments can be released from the solid support using chemical cleavage. The glycopolypeptides can be cleaved with a protease such as trypsin to generate glycopeptide fragments. The method can be used to identify a diagnostic marker for various diseases, as described herein, including but not limited to cancer.

As disclosed herein, the methods described herein and above can optionally be performed with the inclusion of a detergent. The methods described herein and above can also optionally be performed with the inclusion of a quencher. Thus, the methods can optionally be performed with the inclusion of a detergent and/or quencher to quench an oxidation reaction. Accordingly, the invention additionally provides a method for identifying glycopolypeptides in a sample. The method can include the steps of adding a detergent to a sample comprising glycopolypeptides; cleaving glycopolypeptides in the sample to generate glycopeptide fragments; adding.an oxidizing agent to derivatize the glycopeptide fragments; adding a quencher to quench the oxidizing agent; immobilizing the derivatized glycopeptide fragments to a solid support; releasing the glycopeptide fragments from the solid support, thereby generating released glycopeptide fragments; analyzing the released glycopeptide fragments using mass spectrometry; and identifying a released glycopeptide fragment. Such a method can further comprise labeling the immobilized glycopeptide fragments with an isotope tag. Such a method can additionally comprise quantifying the amount of the identified glycopeptide fragment. The optional inclusion of a detergent can provide better dissolution of membrane proteins into the aqueous phase and/or facilitate the denaturation of proteins and access to a protease to generate peptide fragments. The inclusion of a quencher to quench an oxidation reaction can provide better recovery of glycopeptide fragments since the oxidation reaction is stopped by the addition of a quenching agent rather than utilizing a step that requires transfer of the sample to a different vessel or a desalting step, with potential losses that occur with sample transfer or desalting, particularly of low abundance glycopolypeptides. The inclusion of a quencher to “remove” the excess oxidizing agent can improve capture yield, save time and facilitate automation for high throughput analysis.

The invention additionally provides a method of identifying a diagnostic marker for a disease. The method can include the steps of adding a detergent to a test sample and control sample comprising glycopolypeptides; cleaving glycopolypeptides from the test sample to generate test glycopeptide fragments; cleaving glycopolypeptides from the control sample to generate control glycopeptide fragments; adding an oxidizing agent to derivatize the glycopeptide fragments; adding a quencher to quench the oxidizing agent; immobilizing the test glycopeptide fragments to a first solid support; immobilizing the control glycopeptide fragments from a control sample to a second solid support; releasing the test glycopeptide fragments and control glycopeptide fragments from the solid supports; analyzing the released glycopeptide fragments; and identifying one or more glycosylated polypeptides having differential glycosylation between the test sample and the control sample. Such a method can further comprise labeling the immobilized glycopeptide fragments on the first and second supports with differential isotope tags on the respective supports.

As described herein, methods of the invention can optionally include a quencher to quench the oxidation reaction of an oxidizing agent. In a particular embodiment, sodium sulphite can be used as a quencher. Although exemplified herein with sodium sulphite, it is understood that any of a number of quenching agents suitable to inhibit or stop a derivitizing reaction such as oxidation can be used in methods of the invention. Exemplary quenching agents include, but are not limited to, a sulfite such as a sulfite.compound or sulfite salt, including sodium sulfite or other salts thereof, a thiosulfate such as a thiosulfate compound or thiosulfate salt, including sodium thiosulfate (Na₂S₂O₃) or other salts thereof, or other agents that can quench an oxidation reaction by reacting with excess oxidizing agent and inactivating the oxidation reaction.

It is understood that modifications which do not substantially affect the activity of the various embodiments of this invention are also provided within the definition of the invention provided herein. Accordingly, the following examples are intended to illustrate but not limit the present invention.

### EXAMPLE I

### Quantitative Analysis of Glycopeptides

This example describes purification of glycopeptides and differential labeling with isotope tags.

An embodiment of a method of the invention is schematically illustrated in FIG. 1. The method can include the following steps: (1) Glycoprotein oxidation: Oxidation, for example, with periodate, converts the cis-diol groups of carbohydrates to aldehydes (FIG. 2); (2) Coupling: The aldehydes react with hydrazide groups immobilized on a solid support to form covalent hydrazone bonds (FIG. 2). Non-glycosylated proteins are removed; (3) Proteolysis: The immobilized glycoproteins are proteolyzed on the solid support. The non-glycosylated peptides are removed by washing and can be optionally collected for further analysis, whereas the glycosylated peptides remain on the solid support; 4) Isotope labeling: The α amino groups of the immobilized glycopeptides are labeled with isotopically light (d0, contains no deuteriums) or heavy (d4, contains four deuteriums) forms of succinic anhydride after the ε-amino groups of lysine are converted to homoarginine (FIG. 3); (5) Release: Formerly N-linked glycopeptides are released from the solid-phase by PNGase F treatment; (6) Analysis: The isolated peptides are identified and quantified using microcapillary high performance liquid chromatography electrospray ionization tandem mass spectrometry (μLC-ESI-MS/MS) or μLC separation followed by matrix-assisted laser desorption/ionization (MALDI) MS/MS. The data are analyzed by a suite of software tools.

Proteins from a sample, for example, a complex biological sample, were changed to buffer containing 100 mM NaAc, 150 mM NaCl, pH 5.5 (coupling buffer). Sodium periodate solution at 15 mM was added to the samples. The cap was secured and the tube was covered with foil. The sample was rotated end-over-end for 1 hour at room temperature. The sodium periodate was removed from the samples using a desalting column (Econo-Pac 10DG column). Hydrazide resin (Bio-Rad; Hercules Calif.) equilibrated in coupling buffer was added to the sample (1 ml gel/5 mg protein). The sample and resin were capped securely and rotated end-over-end for 10-24 hours at room temperature.

After the coupling reaction was complete, the resin was spun down at 1000×g for 10 min, and non-glycoproteins were washed away extensively by washing the resin 3 times with an equal volume of 8M urea/0.4M NH₄HCO₃. The proteins on the resin were denatured in 8M urea/0.4M NH₄HCO₃ at 55° C. for 30 min, followed by 3 washes with the urea solution. After the last wash and removal of the urea buffer, the resin was diluted 4 times with water. Trypsin was added at a concentration of 1 μg of trypsin/100 μg of protein and the bound proteins digested at 37° C. overnight. If desired, the peptides can be reduced by adding 8 mM TCEP (Pierce, Rockford Ill.) at room temperature for 30 min, and alkylated by adding 10 mM iodoacetamide at room temperature for 30 min. The trypsin released peptides were removed and collected for labeling with ICAT™ reagent or other tagging reagent, if desired. The resin was washed with an equal volume of 1.5 M NaCl 3 times, 80% acetonitrile (MeCN)/0.1% trifluoroacetic acid (TFA) 3 times, 100% methanol 3 times, and 0.1 M NH₄HCO₃ 6 times. N-linked glycopeptides were released from the resin by digestion with peptide-N-glycosidase F (PNGase F) overnight. The resin was spun and the supernatant was saved. O-linked glycopeptides can be released from the resin by using combination of neuraminidase/O-glycosidase. The resin was washed twice with 80% MeCN/0.1% TFA and combined with the supernatant. The peptides were dried and resuspended in 0.4% acetic acid for LC-MS/MS analysis.

Alternatively, the glycopeptides can be released from the resin chemically. The N-linked glycopeptide can be released by hydrazinolysis. Glycopeptides are dried in a desiccator over P₂O₅ and NaOH. The reaction is carried out in an air-tight screw-cap tube using anhydrous hydrazine. The reaction is carried out at 100° C. for about 10 hours using a dry heat block. The release of O-linked glycopeptide is carried out in 50 mM NaOH containing 1 M NaBH₄ at 55° C. for about 18 h.

For isotopic labeling of glycopeptides with succinic anhydride (FIG. 3), the glycopeptides on the beads were washed twice with 15% NH₄OH in water (pH>11). Methylisourea at 1 M in 15% NH₄OH (NH₄OH/H₂O=15/85 v/v) was added in 100 fold molar excess over amine groups and incubated at 55° C. for 10 minutes. Beads were then washed twice with water, twice with dimethylformamide (DMF)/pyridine/H₂O=50/10/40 (v/v/v) and resuspended in DMF/pyridine/H₂O=50/10/40 (v/v/v). Succinic anhydride solution was added to a final concentration of 2 mg/ml. The sample was incubated at room temperature for 1 hour, followed by washing three times with DMF, three times with water, and six times with 0.1M NH₄HCO₃. The peptides were released from the beads using PNGase F as describe above.

Alternatively, the glycopeptides can be labeled with other reagents at amine groups of glycopeptides while the peptides are still conjugated to the hydrazide beads. A list of chemicals that have been tested and proved to be able to label the amino groups is listed in FIG. 3. The structures of labeled peptide are listed at the right column. Once the glycopeptides were labeled isotopically, PNGase F was added to release the peptides from the solid support and analyzed by mass spectrometry.

For isotopic labeling of glycopeptides with Phe (see FIG. 9), 0.22 M of Boc-d0-Phe-OH (Nova Biochem) or Boc-d5-Phe-OH (CDN Isotopes) were dissolved in anhydrous, N,N-dimethyformamide. 1,3-Diisopropylcarbodiimide was added to a final concentration of 0.2 M. The reaction was carried out at room temperature for 2 hours. The glycopeptides on the beads were washed with 0.5 M NaHCO₃ three times and resuspended to a 50% slurry. The same volume of Boc-Phe-anhydride was added to the glycopeptides on the beads, and the beads were incubated at room temperature for 30 min. The beads were washed with 80% MeCN/0.1% TFA three times and dried. The Boc protection group was removed by incubating with TFA for 30 min at room temperature. The beads were washed with glycosidase buffer, followed by release of the labeled glycopeptides with glycosidases, as described above.

This example describes purification of glycopeptides and differential labeling with an isotope tag.

### EXAMPLE II

### Quantitative Glycopeptide Profiling in Human Blood Serum

This example describes profiling of glycoproteins in human blood serum.

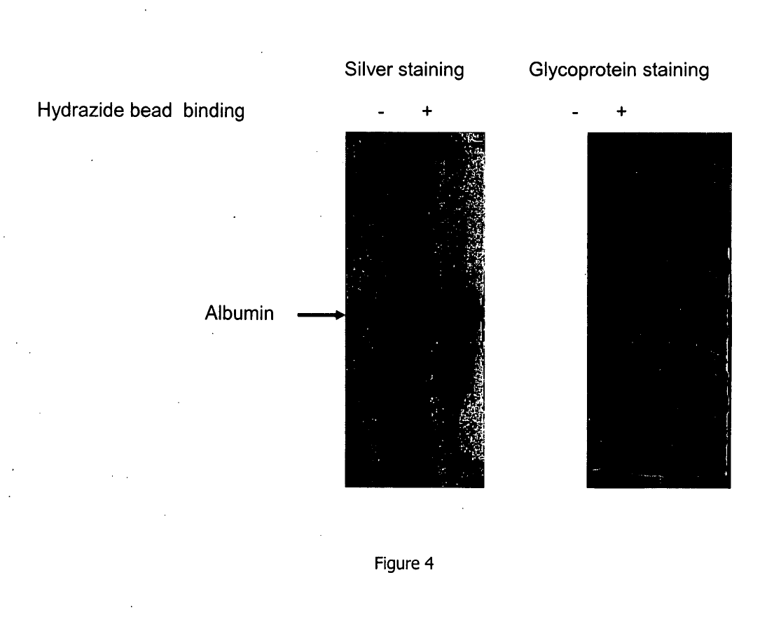

To assess the potential of the glycopeptide capture method for serum protein profiling, the specificity and efficiency of conjugation was first determined. Human serum proteins were coupled to the hydrazide beads. Identical aliquots (1 μl) were removed from the sample before (“−beads”) or after capture of glycoproteins to hydrazide resin (“+beads”). The samples were separated by 9% sodium dodecyl sulfate-polyacrylamide gel electrophoresis (SDS-PAGE) and stained with silver (total protein stain) or with a glycoprotein-staining reagent (FIG. 4).

Isolation of glycopolypeptides was performed essentially as described in Example I. For analysis of serum samples, 2.5 ml of human serum (200 mg total protein) were changed to buffer containing 100 mM NaAc, 150 mM NaCl, pH 5.5 using a desalting column (Bio-Rad). Sodium periodate solution at 15 mM was added to the samples. The cap was secured and the tube was covered with foil. The sample was rotated end-over-end for 1 hour at room temperature. The sodium periodate was removed from the samples using a desalting column. A 50 μl aliquot of the sample was taken before coupling the sample. To the sample was added 8 ml of coupling buffer equilibrated hydrazide resin (Bio-Rad). The sample and resin were capped securely and rotated end-over-end for 10-24 hours at room temperature. After the coupling reaction was complete, the resin was spun down at 1000×g for 10 min, and non-glycoproteins in the supernatant were removed. A 50 μl aliquot of the post conjugation sample was taken.

A portion of each of the aliquots taken before and after coupling (1 μl) was analyzed on a 9% sodium dodecyl sulfate-polyacrylamide gel electrophoresis (SDS-PAGE) gel and stained. For total proteins and glycoproteins, silver staining or GelCode Glycoprotein staining reagent, respectively, were used to determine the specificity and efficiency of glycoprotein isolation.

As shown in FIG. 4, the supernatant prior to addition to the beads (−) contains a number of proteins, many of which stain as glycoproteins (right panel, “−” lane). After addition and incubation with the beads, most of the glcyopolypeptides are removed (left and right panels, “+” lanes). These results show that the hydrazide beads efficiently bind the glcyopolypeptides from the serum sample. Note that the major serum protein, albumin, remained in the supernatant (left panel, “+” lane) and was not stained with the glycoprotein stain (right panel, “−” lane). Thus, albumin does not appear to be glycosylated. Since albumin is the major serum protein (>50%), the use of carbohydrate-specific binding provides a method to efficiently analyze low abundance, glycosylated polypeptides present in serum.

The following is apparent from the experiment shown in FIG. 4. First, as expected, the serum sample contains a considerable amount of glycosylated proteins (glycoprotein stain, “−beads” lane). Second, the majority of the protein bands were essentially depleted by the coupling reaction (silver stained bands “+/−beads” lanes). Third, as far as could be determined from the different staining intensities of the two staining methods used, glycosylated proteins were quantitatively depleted and bands containing glycosylated proteins were preferentially removed by the coupling reaction. Fourth, the major band representing serum albumin was not depleted by the coupling reaction and did not stain with the glycoprotein-staining reagent. Collectively, these results show that the hydrazide beads bind the oxidized glycoproteins from the serum sample efficiently and specifically. They also show that the major serum protein, albumin, predominantly remained in the supernatant (left panel, “+beads” lane) and was not stained with the glycoprotein stain (right panel, “+/−beads” lane). The use of carbohydrate-specific isolation of serum glycoproteins therefore provides a more economical, simpler and more reproducible method for serum albumin removal than the affinity depletion methods commonly used. Since the present method is also compatible with the immobilization of denatured proteins, it reduces the possibility that the selective removal of albumin also removes albumin-associated proteins.

Non-specific proteins bound to the resin were washed away extensively by washing the resin 3 times with an equal volume of 8M urea/0.4M NH₄HCO₃. The proteins on the resin were denatured in 8M urea/0.4M NH₄HCO₃ at 55° C. for 30 min, followed by 3 washes with the urea solution. After the last wash and removal of the urea buffer, the resin was diluted 4 times with water. Trypsin was added at a concentration of 1 μg of trypsin/100 μg of protein and digested at 37° C. overnight. The trypsin released peptides were removed by washing the resin with an equal volume of 1.5 M NaCl for 3 times, 80% MeCN/0.1% TFA for 3 times, 100% methanol for 3 times, and 0.1 M NH₄HCO₃ for 6 times. N-linked glycopeptides were released from the resin by digestion with PNGase F at 37° C. overnight. The resin was spun and the supernatant saved. The resin was washed twice with 80% MeCN/0.1% TFA and combined with the supernatant. The resin was saved for O-linked glycopeptide release later.

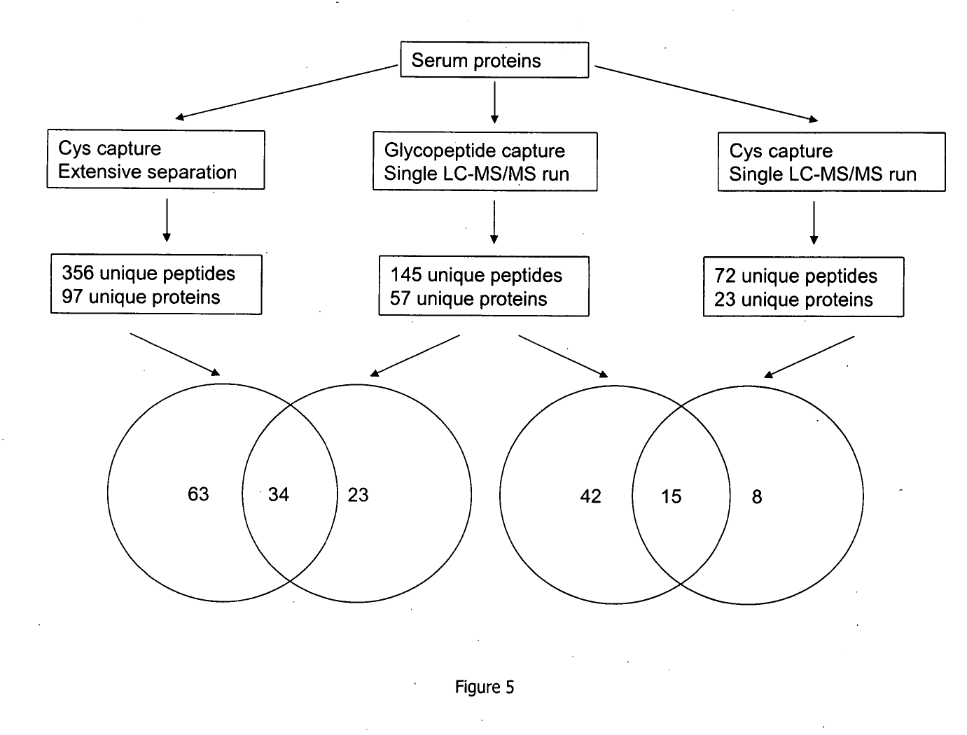

The peptides were dried in 17 tubes, and one tube was resuspended 50 μl of 0.4% acetic acid. A 3 μl aliquot of the sample (from 9 μl of serum) was loaded on a capillary column for μLC-MS/MS analysis. CID spectra were searched against a human database using SEQUEST (Eng et al., J. Am. Soc. Mass. Spectrom. 5:976-989 (1994)) to identify the glycopeptides and glycoproteins (FIG. 5, middle panel).

To determine whether the reduced peptide sample complexity achieved by glycopeptide capture and release allowed the identification of more serum proteins compared to conventionally prepared control samples if comparable μLC-MS/MS protocols were applied, the number of glycopeptides and glycoproteins identified as described above was compared to the number of serum proteins identified from other methods using the same μLC-MS/MS protocols. Control samples were generated by selectively isolating cysteine-containing peptides using the ICAT reagent method (Gygi et al., Nat. Biotechnol. 17:994-999 (1999)). These were analyzed either using the same μLC-ESI-MS/MS method as for the analysis of the peptides isolated by the glycopeptide capture method (FIG. 5, right panel) or via extensive, three dimensional (cation exchange/biotin affinity/reverse phase liquid chromatography (RP-LC)) in which the peptide mixture was fractionated into 17 cation exchange fractions that were sequentially analyzed by μLC-ESI-MS/MS (FIG. 5, left panel; Han et al., Nat. Biotechnol. 19:946-951 (2001)).

Using a single μLC-ESI-MS/MS run requiring approximately two hours of mass spectrometer time, 145 unique peptides mapping to 57 unique serum proteins were identified with the glycopeptide capture method (2.5 peptides/protein). When comparable MS methods were applied for the analysis of cysteine tagged peptides, 72 unique peptides mapping to 23 unique proteins were identified, of which 15 were also identified via the glycopeptide capture method (FIG. 5, right panel). Using the extensive peptide separation protocol for the analysis of cysteine tagged peptides that required approximately 34 hours of mass spectrometer time, 356 unique peptides mapping to 97 serum proteins were identified. Of the 57 proteins isolated by the glycopeptide capture method and identified by single dimensional LC-MS/MS, 23 proteins were not seen by the extensive μLC-ESI-MS/MS based protocol of cysteine tagged peptides (FIG. 5, left panel). These data demonstrate the increased efficiency of serum analysis provided by the glycopeptide capture method.

As the current “gold standard method” for serum protein analysis is based on high resolution two-dimensional electrophoresis (2DE) and MS, the number of proteins identified by a single LC-MS/MS analysis of peptides isolated by the glycopeptide capture method was also related with the number of proteins that are annotated in the most up-to-date 2DE plasma protein map on SWISS-2DPAGE (us.expasy.org/cgi-bin/get-ch2d-table.pl). The 2DE map identifies 58 unique proteins from 626 detected spots. Of these, 270 spots represent 8 different forms of immunoglobulin chains. Glycopeptide capture and single dimensional LC-MS/MS analysis identified 57 proteins, of which 7 are different immunoglobulin chains and 16 proteins are not included in SWISS-2DPAGE. Four major conclusions can be drawn that are relevant for assessing the potential of each method for serum protein profiling, even though, for reasons of sample and experimental variability, the data obtained from the three methods are not directly comparable. First, both the 2DE/MS based method and the cysteine tagging method are substantially limited by the presence of a number of high abundance proteins (that is, the “top down” problem in its extreme), which include the five major plasma proteins representing more than 80% of the total plasma protein mass (albumin, α-1-antitrypsin, β-2-macroglobulin, transferrin, and γ-globulins). When the cysteine tagged peptides were analyzed, the mass spectrometer spent over one third of the acquisition time on CID spectra of albumin (39% of peptides identified by the cysteine tagging method were from albumin). In contrast, the glycopeptide capture method selected against albumin with only 1% of peptides identified from albumin.

Second, proteins that were not identified by either of the traditional methods were readily identified following glycopeptide capture (FIG. 5). This attests to the potential of the glycopeptide capture method to achieve deeper serum protein coverage within a dramatically reduced data acquisition time. The limited diversity of the proteins analyzed by the traditional methods is further illustrated by the observation that of the 63 proteins that were only identified using cysteine reactive tags, 18 were different immunoglobulins. The glycopeptide capture method identified only peptides from the constant region of immunoglobulin and thus limited the number of immunoglobulin-derived peptides (7 immunoglobulin chains identified by the glycopeptide capture method, which were also identified by the cysteine tagging method).

Third, the glycopeptide capture method reduced the sample complexity; an average of 2.5 peptides per protein were detected. Fourth, the presence of the N-glycosylation sequence motif in the identified peptides provided further validation of specific isolation and increased the confidence in database searching results. Therefore, the reduction in sample complexity achieved by the glycopeptide capture method provides a substantial advance for the analysis of blood serum and other body fluids of similar protein composition.

Peptides isolated from 2.5 ml of serum using the glycopeptide capture method were firther separated by cation exchange fractionation (Han et al. Nat. Biotechnol. 19:946-951 (2001)). Four of seventeen tubes containing peptides released from hydrazide resin as described above (equivalent of 600 μl serum) were separated by cation exchange chromatography to 38 fractions and resuspended in 20 pl of 0.4% acetic acid solution. A 5 μl aliquot of each fraction was loaded on a capillary column for μLC-MS/MS analysis. CID spectra were searched against a human database using SEQUEST (Eng et al., J. Am. Soc. Mass. Spectrom. 5:976-989 (1994)) to identify the glycopeptides and glycoproteins.

A large number of glycoproteins and their glycopeptides from a human serum sample were released with PNGase F. A total 1011 proteins identified had a protein probability score of at least 0.5. Based on the distribution of sensitivity and error rate at different protein and peptide probability score (Keller et al., Anal. Chem. 74:5383-5392 (2002)), there were 832 correctly identified proteins with a protein probability score of at least 0.5 (Table 2).

TABLE 2
Estimated sensitivity, error rates, number of correct and incorrect
proteins with different protein probability score.

| | | | Number of | Number of |
|---|---|---|---|---|
| Minimum | | error | correct | incorrect |
| probability | sensitivity | rate | proteins | proteins |
| 1.00 | 0.264 | 0.000 | 288 | 0 |
| 0.99 | 0.291 | 0.001 | 318 | 0 |
| 0.98 | 0.315 | 0.002 | 344 | 1 |
| 0.97 | 0.331 | 0.004 | 362 | 1 |
| 0.96 | 0.344 | 0.005 | 375 | 2 |
| 0.95 | 0.356 | 0.007 | 388 | 3 |
| 0.90 | 0.417 | 0.019 | 455 | 9 |
| 0.80 | 0.522 | 0.049 | 570 | 29 |
| 0.70 | 0.607 | 0.084 | 662 | 61 |
| 0.60 | 0.684 | 0.126 | 746 | 108 |
| 0.50 | 0.762 | 0.177 | 832 | 179 |
| 0.40 | 0.835 | 0.235 | 911 | 279 |
| 0.30 | 0.907 | 0.304 | 990 | 432 |
| 0.20 | 1.000 | 0.406 | 1091 | 746 |

These results show that the glycopeptide capture method also removes albumin from the analysis of serum proteins, thereby allowing the analysis of less abundant serum proteins. The methods allowed the identification of a number of serum proteins that were not easily identified with other methods.

Blood serum is a complex body fluid that contains enormous information about body health. When blood circulates through the body, proteins secreted from cells, shredded from cell surface proteins, and released from dead cells from all tissues are deposited to the blood serum. Blood serum is also the most easily accessible specimen for diagnostic purpose. DNA array technology is not capable of analyzing serum samples since there is not a particular tissue sample from which to extract RNA. The analysis of plasma or serum proteins has also been a focus of proteomics. The two-dimensional electrophoretic technique has been used in the analysis of human plasma proteins since 1977 (Anderson and Anderson, Proc. Natl. Acad. Sci. USA 74:5421-5425 (1977)). To date, 289 plasma proteins have been identified using the 2DE method (Anderson and Anderson, Mol. Cell. Proteomics 1:845-687 (2002)). Recently, direct analysis of serum proteins with mass spectrometry was used to analyze proteins in human serum. In this analysis, abundant immunoglobulin proteins were first affinity depleted from serum sample. The resulting peptides were separated by strong cation exchange chromatography into distinct fractions prior to analysis. 490 serum proteins were identified by on-line reversed-phase microcapillary liquid chromatography coupled with ion trap mass spectrometry (Adkins et al., Mol. Cell. Proteomics 1947-1955 (2002)).

While the use of more extensive separation protocols for the formerly N-glycosylated peptides will increase the depth of serum protein coverage, tryptic peptides that are too short or too long to fall within the detection range of the mass spectrometer used will not be identified. This can be overcome, at least in part, by the use of proteases with cleavage specificities different from that of trypsin.

The increased number of serum proteins identified using the glycopeptide capture method compared to other proteomics methods so far shows that the glycopeptide method is an efficient method to analyze serum proteins and has the capacity to identify low abundance proteins as disease biomarkers in serum.

### EXAMPLE III

### Quantitative Profiling of Glycoproteins Secreted by Macrophages

This example describes the preparation of secreted protein sample from stimulated RAW 264.7 mouse monocyte/macrophage cell line.

Briefly, 10⁹ RAW cells were used. On day 1, cells were plated at a density of 2.5×10⁵ cells/cm² with 10 nM phorbol 12-myristate-13-acetate (PMA). On day 2, the media was removed, and new media was added without PMA. On day 3, the cells were washed three times with serum-free media.

Lipopolysaccharide (LPS) was added as stimulant to the experimental cells with serum-free, PMA-free media. The cells were incubated at 37° C. for 4 hours. The supernatant was removed, and the cells were centrifuged at 3,000×g for 5 minutes to remove cells and large debris. The supernatant was centrifuged at 100,000×g for 1 hour to remove debris.

The supernatant was concentrated with an 80 mL Centricon concentrator, with 300 mL concentrated to <1 mL for each condition. The final concentration of proteins was at least 2 mg/mL.

One mg of proteins secreted from unstimulated and stimulated macrophages was changed to buffer containing 100 mM NaAc, 150 mM NaCl, pH 5.5, using a desalting column (Bio-Rad). Sodium periodate solution at 15 mM was added to the samples. The cap was secured and the tube covered with foil. The sample was rotated end-over-end for 1 hour at room temperature. Sodium periodate was removed from the samples using a desalting column. A 50 μl aliquot of the sample was taken before coupling the sample. To the sample was added 0.2 ml of coupling buffer equilibrated hydrazide resin (Bio-Rad). The resin and sample were capped securely and rotated end-over-end for 10-24 hours at room temperature. After the coupling reaction was complete, the resin was spun down at 1000×g for 10 min, and non-glycoproteins in the supernatant were removed. An aliquot of 50 μl of the post conjugation sample was taken. An aliquot of the samples before and after binding to the resin were analyzed on a 9% SDS-PAGE gel and stained for total proteins using silver staining reagent to determine the specificity and efficiency of glycoprotein isolation.

Non-specific proteins bound to the resin were washed away extensively by washing the resin 3 times with an equal volume of 8M urea/0.4M NH₄HCO₃. The proteins on the resin were denatured in 8M urea/0.4M NH₄HCO₃ at room temperature for 30 min, followed by 3 washes with the urea solution. After the last wash and removal of the urea buffer, the resin was diluted 4 times with water. Trypsin was added at a concentration of 1 μg of trypsin/100 μg of protein and digested at 37° C. overnight. The trypsin released peptides were removed by washing the resin with an equal volume of 1.5 M NaCl for 3 times, 80% MeCN/0.1% TFA for 3 times, 100% methanol for 3 times, 0.1 M NH₄HCO₃ for 6 times. N-linked glycopeptides were released from the resin by digest with N-glycosidase at 37° C. overnight. The resin was spin and the supernatant was saved. The resin was washed twice with 80% MeCN/0.1% TFA and combined with the supernatant. The resin was saved for O-linked glycopeptide release later.

The peptides were dried and resuspended in 50 μl of 0.4% acetic acid. 3 μl of sample was loaded on a capillary column for μLC-MS/MS analysis. CID spectra were searched against a mouse database using SEQUEST to identify the glycopeptides and glycoproteins.

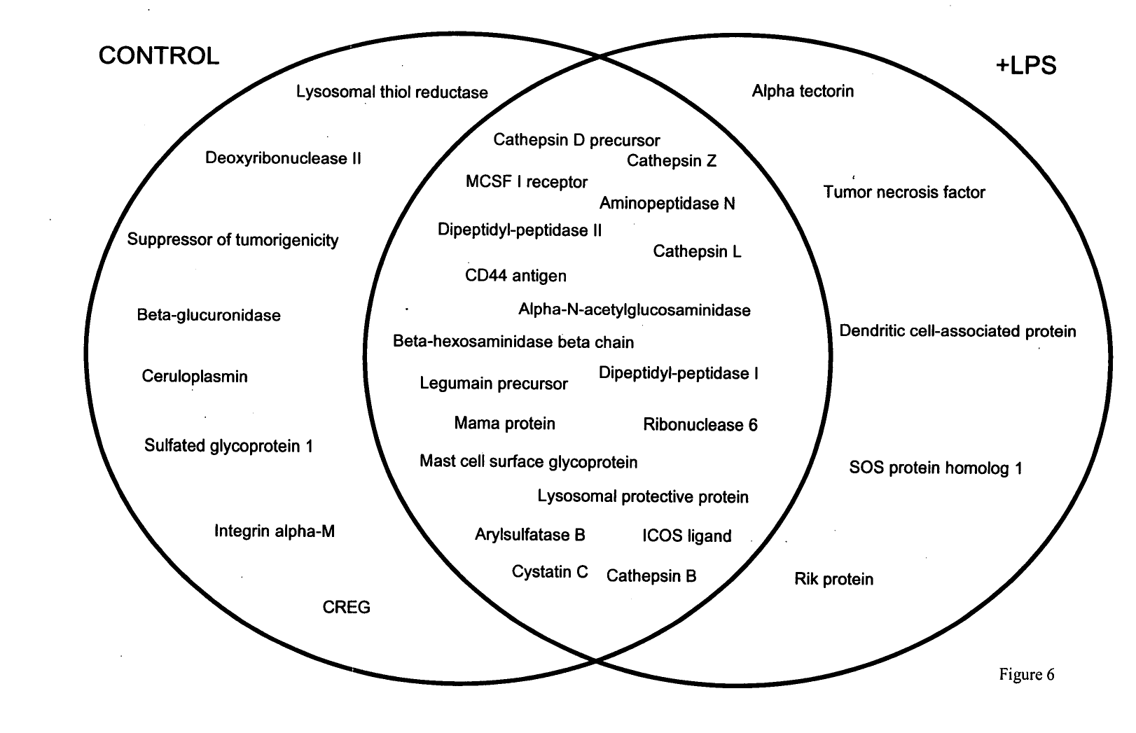

FIG. 6 shows glycoproteins identified from secreted proteins of untreated or LPS-treated RAW macrophage cells. A total of 32 proteins were identified. Nineteen secreted glycosylated proteins were identified in both untreated and treated cells. Eight proteins were identified in untreated cells, and five proteins were identified in treated cells. One of the known macrophage secreted proteins, tumor necrosis factor (TNF), was positively identified in media from RAW cells after LPS treatment. These results show that glycopolypeptides can be selectively isolated from a secreted proteins from cells in an efficient and specific manner.

For isotopic labeling of glycopeptides with succinic anhydride (FIG. 3), the dried peptides released from hydrazide resin were resuspended in DMF/pyridine/H₂O=50/10/40 (v/v/v). Succinic anhydride solution was added to a final concentration of 2 mg/ml. The sample was incubated at room temperature for I hour, followed by purification of peptides using C18 column. Labeled peptides are analyzed by mass spectrometry.

These results demonstrate that glycosylated secreted proteins can be isolated, identified and quantified.

### EXAMPLE IV

### Quantitative Glycopeptide Profiling of Cell Surface Proteins

This example describes profiling of cell surface glycoproteins.

To assess the potential of the glycopeptide capture method for the analysis of cell surface proteins, a crude membrane fraction from the LNCaP prostate cancer epithelial cell line was used to select and identify peptides containing N-linked glycosylation sites (Horoszewicz et al., Prog. Clin. Biol. Res. 37:115-132 (1980)). The released peptides isolated from 60 μg of a crude membrane fraction were analyzed by single dimension μLC-MS/MS and the data were processed.

Briefly, glycopolypeptides were isolated essentially as described in Example I. For the analysis of cell surface proteins, 4 mg of crude membrane fraction from the prostate cancer cell line, LNCAP (grown in RPMI medium supplemented with 10% fetal bovine serum), were dissolved in 1% NP40, 6 M urea, 100 mM Tris buffer, pH 8.3. The buffer was changed to coupling buffer containing 100 mM NaAc, 150 mM NaCl, pH 5.5, using a desalting column (Bio-Rad; Hercules Calif.). Sodium periodate solution was added at 15 mM to the samples. The cap was secured and the tube was covered with foil. The sample was rotated end-over-end for 1 hour at room temperature. The sodium periodate was removed from the samples using a desalting column. A 50 μl aliquot was taken before coupling the sample. To the sample was added 1 ml of coupling buffer equilibrated hydrazide resin (Bio-Rad). The resin and sample were capped securely and rotated end-over-end for 10-24 hours at room temperature.

After the coupling reaction was complete, the resin was spun down at 1000×g for 10 min, and non-glycoproteins were washed away extensively by washing the resin 3 times with an equal volume of 8M urea/0.4M NH₄HCO₃. The proteins on the resin were denatured in 8M urea/0.4M NH₄HCO₃ at 55° C. for 30 min, followed by 3 washes with the urea solution. After the last wash and removal of the urea buffer, the resin was diluted 4 times with water. Trypsin was added at a concentration of 1 μg of trypsin/100 μg of protein and digested at 37° C. overnight. The trypsin released peptides were removed by washing the resin with an equal volume of 1.5 M NaCl for 3 times, 80% MeCN/0.1% TFA for 3 times, 100% methanol for 3 times, and 0.1 M NH₄HCO₃ for 6 times. N-linked glycopeptides were released from the resin by digestion with N-glycosidase overnight. The resin was spun and the supernatant saved. The resin was washed twice with 80% MeCN/0.1% TFA and combined with the supernatant. The resin was saved for O-linked glycopeptide release later.

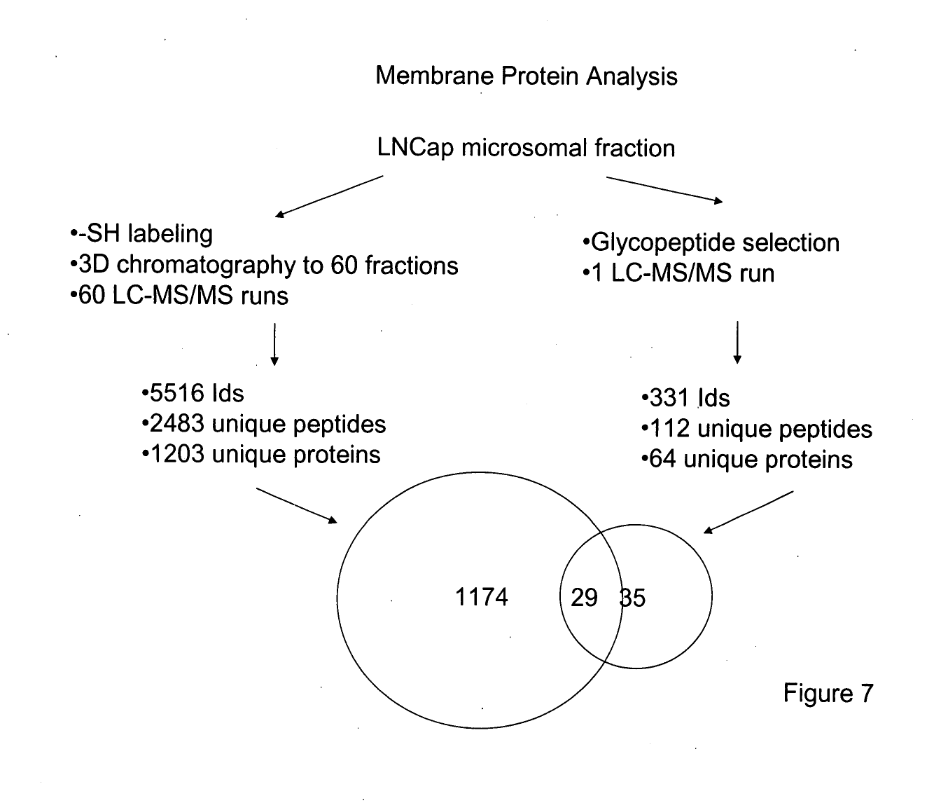

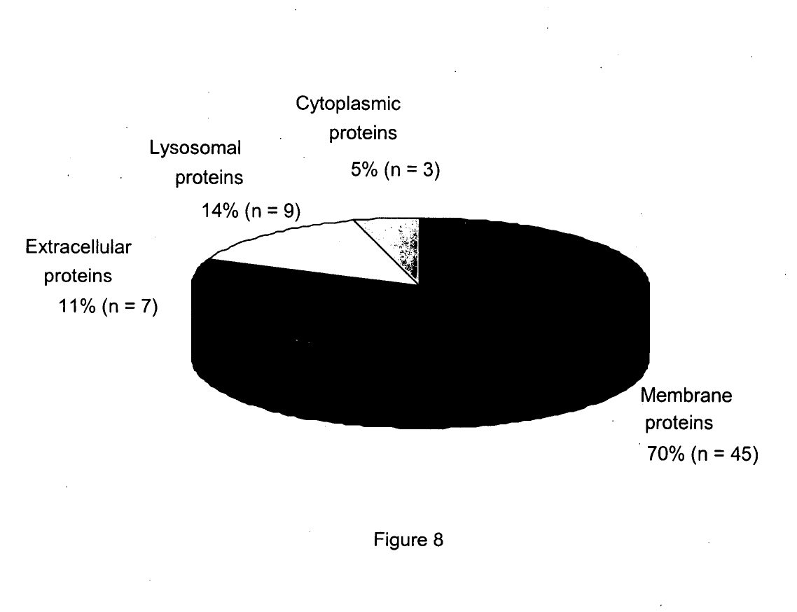

The peptides were dried in 4 tubes, and one tube was resuspended in 50 μl of 0.4% acetic acid. An aliguot of 3 μl of sample (from 60 μg original microsomal proteins) was loaded on a capillary column for μLC-MS/MS analysis. CID spectra were searched against a human database using SEQUEST (Eng et al., supra, 1994) to identify the glycopeptides and glycoproteins (see FIGS. 7 and 8 and Table 3).

As shown in FIG. 7, 1203 unique proteins were identified from the microsomal fraction of LNCaP cells using ICAT reagent followed by intensive 3D chromatography to fractionate the peptide mixture. Using glycopeptide analysis, 64 unique proteins were identified. Of these, 35 glycopolypeptides were identified that were not identified from the total microsome fraction analysis. Table 3 shows glycoproteins and glycopeptides (SEQ ID NOS:64-174) as well as the subcellular localization from a crude membrane fraction of the prostate cancer cell line LNCaP. The glycopeptides contain the conserved N-linked glycosylation motif (NXS/T)(indicated in bold).

The subcellular localization of the identified proteins was further analyzed using information from SWISS-PROT database (www.expasy.org/sprot/) or prediction tool, PSORT II (psort.ims.u-tokyo.ac.jp/). As shown in FIG. 8, of a total of 64 identified glycoproteins, 45 (70%) were bona fide or predicted transmembrane proteins. The non-transmembrane proteins were mostly designated as either extracellular (7 proteins, 11%) or lysosomal (9 proteins, 14%), two cellular compartments known to be enriched for glycoproteins. Only three proteins were assigned as cytoplasmic proteins (5%). Interestingly, two previously identified antigens, melanoma-associated antigen ME491 (CD63) and prostate-specific membrane antigen I (FOH1) were also identified in this experiment. These data indicate a marked improvement in selectivity for cell surface proteins over the analysis of crude microsomal fractions. Over 40% of the proteins identified were not membrane proteins in analysis of crude microsomal fraction (Han et al., Nat. Biotechnol. 19:946-951 (2001)). The data also indicate that proteins of high molecular weight and extreme pI, typically underrepresented in analyses performed using 2DE, are readily identified by this method. This is exemplified by the identification of basement membrane-specific heparan sulfate proteoglycan core protein (gene name SW: PGBM), a 470 kDa extracellular protein, and the acidic (PI=4.39) transmembrane protein signal sequence receptor a subunit (gene name SW: SSRA). These results indicate that the glycopeptide capture method is also effective for the selective analysis of proteins contained in the plasma membrane. Furthermore, proteins that were not detectable in analysis of a total microsome fraction were readily identified (see FIG. 7). These results indicate that the methods can be used to analyze glycopolypeptides not otherwise amenable to analysis of a total microsome protein fraction.

TABLE 3
Subcellular location of glycoproteins identified from LNCap cells

| | | | |
|---|---|---|---|
| Gene Name a | Protein Name | Subcellular Location b | Peptide Sequence c |

| | | | |
|---|---|---|---|
| GP:AB002313_1 | mRNA for KIAA0315 gene | Transmembrane, | K.LHVTLYNCSFGR.S |
| | | ER/Golgi/Plasma membrane a | R.SINVTGQGFSLIQR.F |
| | | | R.TEAGAFEYVPDPTFENFTGGVKK. |
| | | | Q |
| GP:AB033767_1 | BSCv mRNA | Transmembrane, ER | R.AGPNGTLFVADAYK.G |
| | | | K.LLLSSETPIEGKNMSFVNDLTVTQ |
| | | | DGR.K |
| GP:AB045981_1 | hFKBP65 mRNA for FK506 | Extracellular | R.YHYNGSLMDGTLFDSSYSR.N |
| | binding protein | | |
| GP:AF089745_1 | FK506-binding protein (FKBP63) | Transmembrane, | R.YHYNGTFLDGTLFDSSHNR.M |
| | mRNA | ER/Mitochondrial/Cytoplasmic | R.YHYNGTLLDGTLFDSSYSR.N |
| GP:AF302102_1 | costimulatory molecule mRNA | Transmembrane, | R.TALFPDLLAQGNASLR.L |
| | | ER/Golgi/Plasma membrane | |
| GP:AJ245820_1 | mRNA for type I transmembrane | Transmembrane, | R.LLANSSMLGEGQVLR.S |
| | receptor (psk-1 gene) | ER/Golgi/Plasma membrane | |
| GP:AY032885_1 | | Transmembrane, ER/Golgi | K.QVALQTFGNQTTIIPAGGAGYK.V |
| GP:BC001123_1 | Similar to gp25L2 protein | Transmembrane, | R.FTFTSHTPGEHQICLHSNSTK.F |
| | | ER/Golgi/Plasma membrane | |
| GP:BC001615_1 | Similar to hypothetical protein | Transmembrane, | K.IFIFNQTGIEAK.K |
| | FLJ22625 | Cytoplasmic/Vesicles of | |
| | | secretory system | |
| GP:BC001740_1 | | Extracellular | K.AVLVNNITTGER.L |
| | | | R.LQQDVLQFQKNQTNLER.K |
| GP:BC004423_1 | clone MGC:3530 IMAGE:2819660 | Transmembrane, | K.VVMDIPYELWNETSAEVADLK.K |
| | | Nuclear/mitochondrial | |
| GP:BC006786_1 | | Extracellular | K.LNITNIWVLDYFGGPK.I |
| GP:BC007443_1 | Similar to FK506 binding protein | Transmembrane, | K.YHYNASLLDGTLLDSTWNLGK.T |
| | 9 | ER/Mitochondrial/Cytoplasmic | |
| GP:BC010078_1 | serine carboxypeptidase 1 | Transmembrane, | R.KTTWLQAASLLFVDNPVGTGFSY |
| | | Golgi/ER/Mitochondrial | VNGSGAYAK.D |
| GP:BC015678_1 | hypothetical protein GL012 | Mitochondrial/Cytoplasmic | R.CFATTYYLSEGGGLIFRNVTGEPN |
| | | | CRPPTR.G |
| GP:BC016467_1 | | Extracellular | R.YHYNGTLLDGTSFDTSYSK.G |
| GP:D85390_1 | mRNA for gp180-carboxypeptidase | Transmembrane, ER | R.GILNATISVAEINHPVTTYK.T |
| | D-like enzyme | | R.GLVMNYPHITNLTNLGQSTEYR.H |
| | | | R.LLNTTDVYLLPSLNPDGFER.A |
| PIR2:A47161 | Mac-2-binding glycoprotein | Extracellular | R.ALGFENATQALGR.A |
| PIR2:G01447 | | Transmembrane, | R.VFPYISVMVNNGSLSYDHSK.D |
| | | Cytoplasmic/Vesicles of | |
| | | secretory system | |
| PIR2:T42709 | hypothetical protein | Cytoplasmic | R.YHYNCSLLDGTQLFTSHDYGAPQ |
| | DKFZp586I0821 | | EATLGANK.V |
| PIR2:T47140 | hypothetical protein | Transmembrane, Vesicles of | R.YSLNVTYNYPVHYFDGR.K |
| | DKFZp761K1115.1 | secretory system/nuclear | |
| SW:4F2_HUMAN | 4f2 cell-surface antigen heavy | Type II membrane protein b | R.DIENLKDASSFLAEWQNITK.G |
| | chain (4f2hc) | | R.LLIAGTNSSDLQQILSLLESNK.D |
| | | | K.SLVTQYLNATGNR.W |
| SW:ASAH_HUMAN | acid ceramidase | Lysosomal | K.ILAPAYFILGGNQSGEGC*VITR.D |
| | | | R.TVLENSTSYEEAK.N |
| SW:ATNB_HUMAN | sodium/potassium-transporting | Type II membrane protein | R.FKLEWLGNCSGLNDETYGYK.E |
| | atpase beta-1 chain | | R.VLGFKPKPPKNESLETYPVMK.Y |
| | | | K.YLQPLLAVQFTNLTMDTEIR.I |
| SW:ATND_HUMAN | sodium/potassium-transporting | Type II membrane protein | K.LHVGYLQPLVAVQVSFAPNNTGK |
| | atpase beta-3 chain | | .E |
| | | | K.LHVGYLQPLVAVQVSFAPNNTGK |
| | | | EVTVECK.I |
| SW:BASI_HUMAN | basigin precursor (leukocyte | Type I membrane protein | K.ILLTCSLNDSATEVTGHR.W |
| | activation antigen m6) | | K.ITDSEDKALMNGSESR.F |
| SW:BGLR_HUMAN | beta-glucuronidase | Lysosomal | R.LLDAENKVVANGTGTQGQLK.V |
| | | | K.VVANGTGTQGQLK.V |
| SW:C166_HUMAN | cd166 antigen precursor | Type I membrane protein | K.IIISPEENVTLTCTAENQLER.T |
| | (activated leukocyte-cell | | K.LGDCISEDSYPDGNITWYR.N |
| | adhesion molecule) | | R.LNLSENYTLSISNAR.I |
| | | | R.TVNSLNVSAISIPEHDEADEISDEN |
| | | | R.E |
| | | | R.TVNSLNVSAISIPEHDEADEISDEN |
| | | | REK.V |
| SW:CATD_HUMAN | cathepsin d | Lysosomal | K.GSLSYLNVTR.K |
| | | | K.YYKGSLSYLNVTR.K |
| SW:CATL_HUMAN | cathepsin 1 | Lysosomal | K.YSVANDTGFVDIPK.Q |
| SW:CD63_HUMAN | cd63 antigen (melanoma- | Integral membrane protein; | R.QQMENYPKNNHTASILDR.M |
| | associated antigen me491) | Lysosomal | |
| SW:CLUS_HUMAN | clusterin | Extracellular | K.MLNTSSLLEQLNEQFNWVSR.L |
| SW:DRN2_HUMAN | deoxyribonuclease ii (lysosomal | Lysosomal | K.GHHVSQEPWNSSITLTSQAGAVF |
| | dnase ii) | | QSFAK.F |
| SW:DSG2_HUMAN | desmoglein 2 | Type I membrane protein | K.DTGELNVTSILDREETPFFLLTGY |
| | | | ALDAR.G |
| SW:ENPL_HUMAN | endoplasmin | ER | K.HNNDTQHIWESDSNEFSVIADPR. |
| | | | G |
| | | | K.YLNFVKGVVDSDDLPLNVSR.E |
| SW:FOH1_HUMAN | folate hydrolase (prostate- | Type II membrane protein | K.FLYNFTQIPHLAGTEQNFQLAK.Q |
| | specific membrane antigen 1) | | R.GVAYINADSSIEGNYTLR.V |
| | | | K.TYSVSFDSLFSAVKNFTEIASK.F |
| | | | R.VDCTPLMYSLVHNLTK.E |
| | | | K.VPYNVGPGFTGNFSTQK.V |
| SW:GL6S_HUMAN | n-acetylglucosamine-6-sulfatase | transmembrane, Lysosomal | K.TPMTNSSIQFLDNAFR.K |
| | | | K.YYNYTLSINGK.A |
| SW:GLCM_HUMAN | glucosylceramidase | transmembrane, lysosomal | R.DLGPTLANSTHHNVR.L |
| | | | R.MELSMGPIQANHTGTGLLLTLQP |
| | | | EQK.F |
| | | | R.RMELSMGPIQANHTGTGLLLTLQ |
| | | | PEQK.F |
| | | | R.TYTYADTPDDFQLHNFSLPEEDTK |
| SW:GLG1_HUMAN | golgi sialoglycoprotein mg-160 | Type I membrane protein, | R.DIVGNLTELESEDIQIEALLMR.A |
| | (cysteine-rich fibroblast growth | Golgi | |
| | factor receptor) | | |
| SW:HEXB_HUMAN | beta-hexosaminidase beta chain | Lysosomal | K.LDSFGPINPTLNTTYSFLTTFFK.E |
| SW:ITAV_HUMAN | integrin alpha-v | type I membrane protein | R.TAADTTGLQPILNQFTPANISR.Q |
| SW:LDLR_HUMAN | low-density lipoprotein receptor | type I membrane protein | R.LTGSDVNLLAENLLSPEDMVLFH |
| | | | NLTQPR.G |
| SW:LMG1_HUMAN | laminin gamma-1 chain | transmembrane, extracellular | K.LLNNLTSIK.I |
| SW:LMP1_HUMAN | lysosome-associated membrane | Type I membrane protein | R.GHTLTLNFTR.N |
| | glycoprotein 1 | | K.SGPKNMTFDLPSDATVVLNR.S |
| SW:LMP2_HUMAN | lysosome-associated membrane | Type I membrane protein | K.IAVQFGPGFSWIANFTK.A |
| | glycoprotein 2 | | K.WQMNFTVR.Y |
| SW:LU_HUMAN | lutheran blood group glyco- | Type I membrane protein | R.TQNFTLLVQGSPELK.T |
| | protein | | |
| SW:LYAG_HUMAN | lysosomal alpha-glucosidase | Lysosomal | R.GVFITNETGQPLIGK.V |
| SW:LYII_HUMAN | lysosome membrane protein ii | Type II membrane protein; | K.CNMINGTDGDSFHPLITK.D |
| | | lysosomal | R.TMVFPVMYLNESVHIDK.E |
| | | | R.TMVFPVMYLNESVHIDKETASR.L |
| SW:MA2B_HUMAN | lysosomal alpha-mannosidase | transmembrane, Lysosomal | R.LEHQFAVGEDSGRNLSAPVTLNL |
| | | | R.D |
| SW:MPRI_HUMAN | cation-independent mannose-6- | Type I membrane protein; | R.ATLITFLCDRDAGVGFPEYQEEDN |
| | phosphate receptor | lysosomal | STYNFR.W |
| | | | R.HGNLYDLKPLGLNDTIVSAGEYT |
| | | | YYFR.V |
| | | | K.IKTNITLVCKPGDLESAPVLR.T |
| | | | R.SLLEFNTTVSCDQQGTNHR.V |
| | | | K.TNITLVCKPGDLESAPVLR.T |
| SW:NCM2_HUMAN | neural cell adhesion molecule 2 | Type I membrane protein | K.LVLPAKNTTNLK.T |
| | | | R.SHGVQTMVVLNNLEPNTTYEIR.V |
| SW:NEP_HUMAN | neprilysin | Type II membrane protein | R.SCINESAIDSR.G |
| | | | K.VMELEKEIANATAKPEDR.N |
| SW:NICA_HUMAN | nicastrin | Type I membrane protein | R.TSLELWMHTDPVSQKNESVR.N |
| SW:OXRP_HUMAN | 150 kda oxygen-regulated protein | transmembrane, ER | R.AEPPLNASASDQGEK.V |
| | | | K.LGNTISSLFGGGTTPDAKENGTDT |
| | | | VQEEEESPAEGSK.D |
| | | | R.LSALDNLLNHSSMFLK.G |
| | | | R.QTVHFQISSQLQFSPEEVLGMVLN |
| | | | YSR.S |
| | | | R.VFGSQNLTTVK.L |
| | | | K.VINETWAWK.N |
| | | | K.VINETWAWKNATLAEQAK.L |
| SW:PGBM_HUMAN | basement membrane-specific | transmembrane, extracellular | R.LPQVSPADSGEYVCRVENGSGPK. |
| | heparan sulfate proteoglycan | surface | E |
| | core protein | | |
| SW:PPT_HUMAN | palmitoyl-protein thioesterase | Extracellular/vacuolar/mito- | K.FLNDSIVDPVDSEWFGFYR.S |
| | | chondrial | |
| SW:PTK7_HUMAN | tyrosine-protein kinase-like 7 | Type I membrane protein | R.MHIFQNGSLVIHDVAPEDSGR.Y |
| | precursor (colon carcinoma | | |
| | kinase-4) | | |
| SW:SAP_HUMAN | P07602 h proactivator poly- | transmembrane, lysosomal | K.DVVTAAGDMLKDNATEEEILVYL |
| | peptide | | EK.T |
| | | | R.NLEKNSTKQEILAALEK.G |
| SW:SE1L_HUMAN | sel-1 homolog precursor | Integral membrane protein | K.GQTALGFLYASGLGVNSSQAK.A |
| | (suppressor of lin-12-like | | |
| | protein) | | |
| SW:SPHM_HUMAN | n-sulphoglucosamine | Lysosomal | R.DAGVLNDTLVLFTSDNGIPFPSGR. |
| | sulphohydrolase | | T |
| | | | R.NALLLLADDGGFESGAYNNSAIA |
| | | | TPHLDALAR.R |
| SW:SSRA_HUMAN | signal sequence receptor alpha | Type I membrane protein; ER | R.YPQDYQFYIQNFTALPLNTVVPPQ |
| | subunit | | R.Q |
| SW:SSRB_HUMAN | signal sequence receptor beta | Type I membrane protein; ER | K.AGYFNFTSATITYLAQEDGPVVIG |
| | subunit | | STSAPGQGGILAQR.E |
| | | | R.IAPASNVSHTVVLRPLK.A |
| SW:TPP1_HUMAN | tripeptidyl-peptidase i | Lysosomal | K.FLSSSPHLPPSSYFNASGR.A |
| SWN:STM1_HU- | stromal interaction molecule 1 | Type I membrane protein cell | R.LAVTNTTMTGTVLK.M |
| MAN | | surface | |

a: Gene name is from human NCBI protein database (www.ncbi.nlm.nih.gov).
b: Subcellular locations in italic letter are predicted by PSORT, and b) in regular letter are from SWISS-PROT
c: The consensus motif for N-linked glycosylation is highlighted.

The total number of proteins identified in this experiment is relatively small but consistent with the number of unique proteins identified from complex samples using LC-MS/MS without extensive separation. Because of the “top down” mode of precursor ion selection in the mass spectrometer, the most abundant proteins are preferentially identified. To identify a higher number of proteins, the sample would have to be more extensively fractionated prior to mass spectrometric analysis.

The method provides for quantitative profiling of glycoproteins or glycopeptides. The method allows the identification and quantification of glycoproteins containing N-linked carbohydrate in a complex sample and the determination of the site(s) of glycosylation. The selectivity of the method makes it ideally suited for the analysis of samples that are enriched in glycosylated proteins. These include cell membranes, body fluids and secreted proteins. Such samples are of great biological and clinical importance, in particular for the identification of diagnostic biomarkers and targets for immunotherapy or pharmacological intervention.

By combining this method with the cysteine tagging method using ICAT reagents (Gygi et al., supra,, 1999), the occupancy of individual N-linked glycosylation sites and changes thereof can also be determined. This is of particular interest in studies in which changes of glycosylation occupancy are suspected, as exemplified by patients with Type I Congenital Disorders of glycosylation, in which the pathway of N-linked glycosylation is deficient (Aebi and Hennet, Trends Cell Biol. 11:136-141 (2001)).

The selectivity of the method also substantially reduces the complexity of the peptide mixture if complex protein samples are being analyzed because glycoproteins generally only contain a few glycosylation sites. The method is focused on the analysis of N-linked glycosylation sites. Analogous strategies can be devised to also analyze O-glycosylated peptides and in fact, a protein sample, once immobilized on a solid support, can be subjected to sequential N-linked and O-linked glycosylation peptide release, thus further increasing the resolution of the method and the information contents of the data obtained by it. Therefore, the method has wide applications in proteomics research and diagnostic applications.

These results show that membrane glycopolypeptides can be readily analyzed. Furthermore, glycopolypeptides that were not detectable in analysis of a total microsome fraction were readily identified (see FIG. 7). These results indicate that the methods can be used to analyze glycopolypeptides not otherwise amenable to analysis of a total microsome protein fraction. Also note that the method simplifies the analysis and focus on proteins located in plasma membrane and extracellular surface, which have therapeutic value for easy drug accessibility and antibody directed therapy.

### EXAMPLE V

### Quantitative Glycopeptide Profiling of Mouse Ascites Fluid

This example describes profiling of glycoproteins from mouse ascites fluid.

Glycopolypeptides were purified essentially as described in Example I. For the analysis of ascites fluid, 20 μl of mouse ascites fluid (600 μg total protein) were changed to buffer containing 100 mM NaAc, 150 mM NaCl, pH 5.5, using a desalting column (Bio-Rad). Sodium periodate solution was added at 15 mM to the samples. The cap was secured and the tube was covered with foil. The sample was rotated end-over-end for I hour at room temperature. The sodium periodate was removed from the samples using a desalting column. An aliquot of 20 μl of coupling buffer equilibrated hydrazide resin (Bio-Rad) was added to the sample. The sample and resin were capped securely and rotated end-over-end for 10-24 hours at room temperature.

After the coupling reaction was complete, the resin was spun down at 1000×g for 10 min, and non-glycoproteins were washed away extensively by washing the resin 3 times with an equal volume of 8M urea/0.4M NH₄HCO₃. The proteins on the resin were denatured in 8M urea/0.4M NH₄HCO₃ at 55° C. for 30 min, followed by 3 washes with the urea solution. After the last wash and removal of the urea buffer, the resin was diluted 4 times with water. Trypsin was added at a concentration of 1 pg of trypsin/100 μg of protein and digested at 37° C. overnight. The trypsin released peptides were removed by washing the resin with an equal volume of 1.5 M NaCl for 3 times, 80% MeCN/0.1% TFA for 3 times, 100% methanol for 3 times, and 0.5 M NaHCO₃ three times, and the resin was resuspended in 20 μl of 0.5 M NaHCO₃, pH 8.0.

For modification of peptides, 0.22 M of Boc-d0-Phe-OH (Nova Biochem) or Boc-d5-Phe-OH (CDN Isotopes) were dissolved in anhydrous N,N-Dimethyformamide. 1,3-Diisopropylcarbodiimide was added to a final concentration of 0.2 M, and the reaction was carried out at room temperature for 2 hours. A 10 μl aliquot of Boc-Phe-anhydride heavy or light forms was added to 10 μl of glycopeptides on the beads and incubated at room temperature for 30 min. The beads were washed with 80% MeCN/0.1% TFA three times, combined and dried. The Boc was removed by incubating with TFA for 30 min at room temperature. The beads. were washed with glycosidase buffer, followed by release of the labeled glycopeptides with N-glycosidases at 37° C. overnight. N-glycopeptides were dried and resuspended in 20 μl of 0.4% of acetic acid. A 2 μl aliquot was analyzed by LC-MS/MS to determine the quantification of N-terminal labeling of glycopeptides by Phe (see FIGS. 9-12).

Mass spectrometry analysis of the peptide by LCQ and searching protein database by Sequest resulted in the identification of N-glycosylated peptides with the conserved N-glycosylation motif NXS/T. More than 50 glycoproteins were identified from 20 μl of mouse ascetic fluid, indicating the method is sensitive and useful for the identification of the glycoproteins from biological samples.

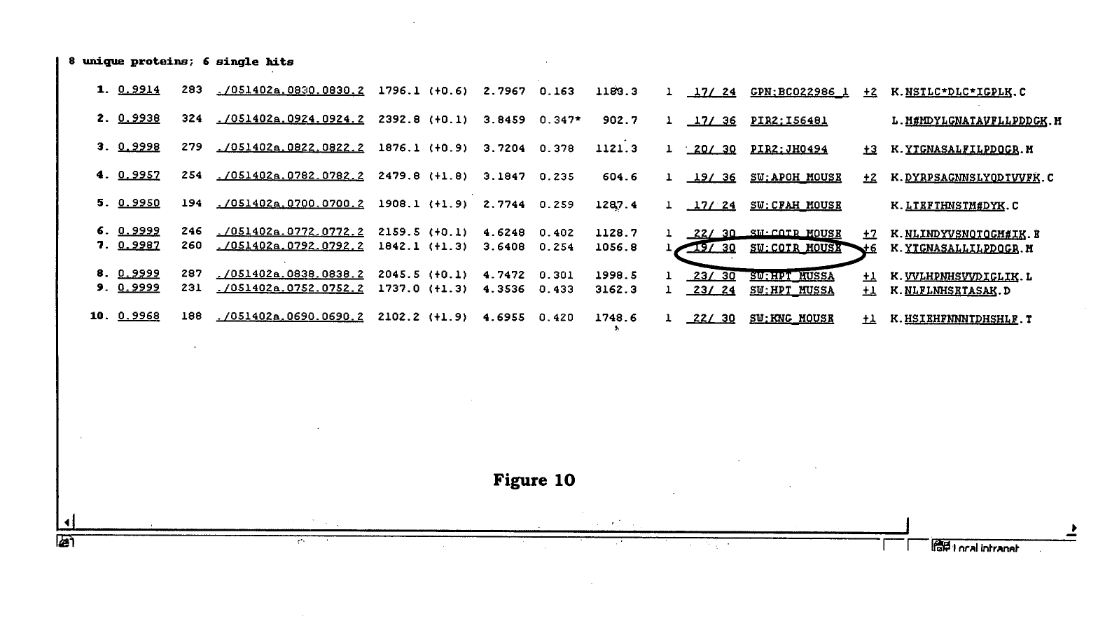

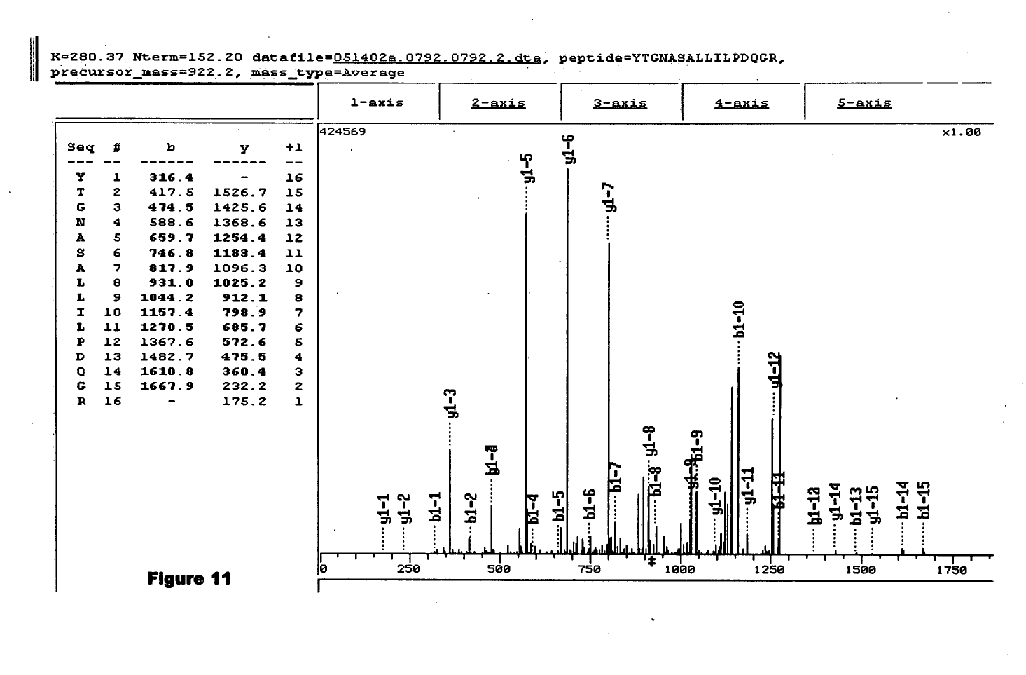

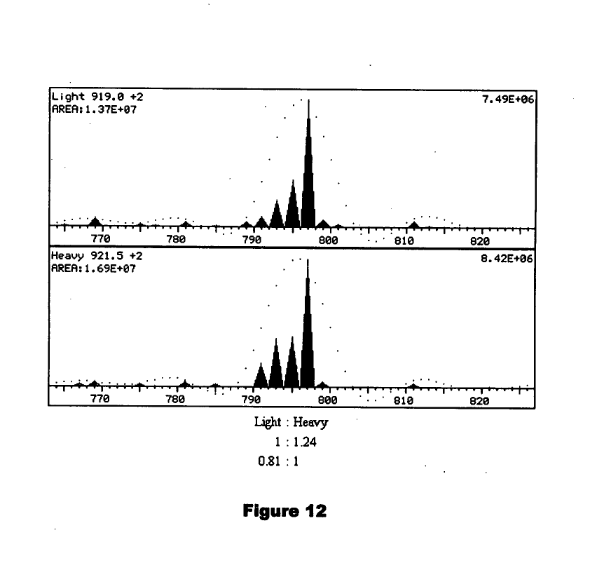

As shown in FIG. 9, isotopic labeling with Phe was performed with two equal amounts of mouse ascites fluid (1 μl), and the formerly N-linked glycopeptides were identified using MS/MS. FIG. 10 shows the list identified peptides after isotopically labeling with Phe. The corresponding collision-induced dissociation (CID) spectrum of one of the identified peptides, indicated by a circle, was shown in FIG. 11.

FIG. 12 shows reconstructed ion chromatograms for the peptide measured in FIG. 11. The ratio of the calculated peak area for the heavy and light form of the isotope tagged peptides was used to determine the relative peptide abundance in the original mixtures (light scan: mass 1837.0; heavy scan: mass 1842.0). The ratio (0.81:1) agreed reasonably well with the expected ratio of 1 to 1.

These results show that glycopolypeptides from complex body fluids can be analyzed, identified and quantified. Using isotope tags, two samples were compared and the relative amount of peptide in the original mixtures was determined, showing that the methods can be used quantitatively.

### EXAMPLE VI

### Quantitative Glycopeptide Analysis of Control Glycoproteins with a Known Ratio

This example describes quantitative analysis of glycoproteins from a pure glycoprotein mix with a known ratio and from two equal amounts of a human serum protein mix.

Two mixtures containing the same three glycoproteins at different amounts were prepared. The proteins were purchased from Calbiochem (San Diego, Calif.). The amount of each protein (μg) in mixture A and B were: α-1-antitrypsin (50, 10), α-2-hs-glycoprotein (10, 30), and α-1-antichymotrypsin (2, 2). Formerly N-linked glycosylated peptides from the two protein mixtures were purified and labeled as described in Example I.

Formerly N-glycosylated peptides were analyzed by μLC-ESI-MS/MS and identified. Table 4 shows the identified sequences (SEQ ID NOS:175-179) and the observed d0/d4 peptide ratio for each identified peptide from two experiments. Of the four identified N-glycosylation sites, three have been described previously (Yoshioka et al., J. Biol. Chem. 261:1665-1676 (1986); Mills et al., Proteomics 1:778-786 (2001); Baumann et al., J. Mol. Biol. 218:595-606 (1991)), while N# in the sequence FN#LTETSEAEIHQSFQH (SEQ ID NO:180) represents a glycosylation site in α-1-antichymotrypsin that has not been described previously. The abundance ratios calculated from the isotopic ratios agreed reasonably with the expected values. These results indicate that the method selectively isolates and quantifies N-linked glycopeptides from mixtures of glycoproteins.

The specific capture of glycoproteins is based on the oxidation of hydroxyl groups on adjacent carbon atoms of carbohydrates to aldehydes by sodium periodate as previously described (Bobbitt, Adv. Carbohydr. Chem. 11, 1-41 (1956)). The aldehydes in turn covalently couple to amine- or hydrazide-containing molecules (Bayer et al., Anal. Biochem. 170:271-281 (1988)). Under the conditions used, the only expected side reaction of sodium periodate oxidation resulting in aldehydes is the oxidation of polypeptides containing a primary amine and a secondary hydroxyl group on adjacent carbon atoms, as exemplified by N-terminal serine residues (Geoghegan and Stroh, Bioconjug. Chem. 3:138-146 (1992)). This constellation is rare in proteins. The attachment of periodate oxidized proteins to hydrazide resin is therefore quite specific for glycoproteins containing N-linked and/or O-linked carbohydrates. Different types of oligosaccharides oxidize at different periodate concentrations and reaction conditions. The conditions used here (15 mM sodium periodate, room temperature for one hour) were chosen to assure oxidation of all types of oligosaccharides with hydroxy groups on adjacent carbon atoms. The enzyme catalyzed release of formerly N-glycosylated peptides by PNGase F provides specificity for N-linked glycopeptides and -N-linked glycosylation sites (Maley et al., Anal. Biochem. 180:195-204 (1989)). PNGase F will not, however, release N-linked oligosaccharides containing core fucosylation.

TABLE 4
Quantitative analysis of glycoproteins in glycoprotein mixture

| | | | | | Expected |
|---|---|---|---|---|---|
| | | | Observed | Protein | Protein |
| | | Glycosylation | Peptide Ratio | Ratio | Ratio |
| Protein Name | Sequences of Identified Peptidesa | Sites | (A/B) | (A/B) | (A/B) |
| α-1-antichymotrypsin | K.FN#LTETSEAEIHQSFQH.L | Novel | 0.69; 0.91 | 1.09 ± 0.39 | 1.00 |
| | F.LSLGAHN#TTLTEILK.G | Known | 1.63; 1.35 | | |
| | L.SISTALAFLSLGAHN#TTLTEIL | Known | 0.88 | | |
| | K.G | | | | |
| α-1-antitrypsin | R.QLAHQSN#STNIFF.S | Known | 6.47; 4.06 | 5.27 ± 1.70 | 5.00 |
| α-2-hs-glycoprotein | K.AALAAFNAQNN#GSNFQLEEI | Known | 0.34; 0.51 | 0.42 ± 0.12 | 0.33 |
| | SR.A | | | | |

It was also determined whether the glycopeptide selection method could be used for detecting quantitative changes in the profiles of N-linked glycopeptides isolated from different samples of human serum. In a proof-of-principle experiment, glycopeptides from two equal amounts of human serum (1 mg total protein) were isotopically labeled with either light (d0) or heavy (d4) forms of succinic anhydride at N-termini after C-terminal lysine residues were converted to homoarginines as described in Example I. The lysine-to-homoarginine conversion facilitated detection by MALDI quadrupole time-of-flight (MALDI-QqTOF) mass spectrometry and the stable isotope tag was incorporated for quantification. After labeling, the beads containing the two samples were combined, and the formerly N-linked glycopeptides were released. A fraction of the sample, equivalent to 1.25 μl of serum, was fractionated to 29 spots on a MALDI plate by RP-LC and analyzed by MALDI-QqTOF MS and MS/MS. The experiment was repeated and analyzed by ESI-QqTOF MS, and the results were comparable to those identified by MALDI-QqTOF MS. Table 5 lists the identified peptides (SEQ ID NOS:181-197), the proteins from which they originated and their observed quantitative ratio from two experiments. Generally, the observed ratios were close to the expected ratio of 1. The differences between the observed and expected ratio ranged between 0%-29% with a mean of 8%. This indicates that the glycopeptide capture method allows reasonable quantification if combined with stable isotope tagging.

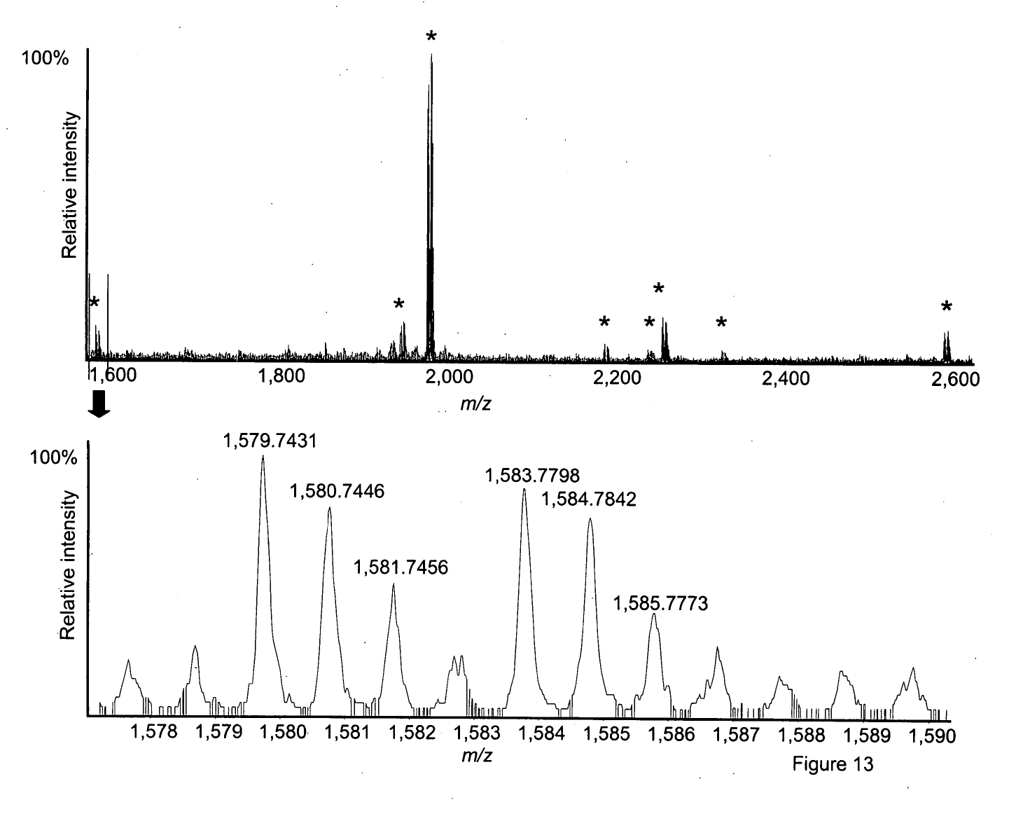

The quantification is further illustrated for a single peptide pair in FIG. 13. A single scan of the mass spectrometer at spot 28 in MS mode identified eight paired signals with a mass difference of four units (indicated with *, FIG. 13). An expansion of the mass range between m/z=1577 and m/z=1590 resolved the natural isotopic distribution of a peptide pair with monoisotopic peaks at 1579.74 and 1583.78, in which the signals had a quantitative ratio of 1.11.

TABLE 5
Quantitative analysis of glycoproteins from two identical serum samples

| | | Sequences of identified | Observed Ratio | Expected | % |
|---|---|---|---|---|---|
| Gene Name a | Protein Names | peptides b | (Mean ± SD) | Ratio | Error |
| GP:AF384856_1 | peptidoglycan recognition protein L | R.GFGVAIVGN#YTAALPTE | 0.95 ± 0.02 | 1 | 5 |
| | | AALR.T | | | |
| GP:M36501_1 | α-2-macroglobulin | Y.VLDYLN#ETQQLTPEIK.S | 0.93 ± 0.03 | 1 | 7 |
| SW:A1AG_HUMAN | α-1-acid glycoprotein 1 | N.LVPVPITN#ATLDQITGK. | 1.05 ± 0.11 | 1 | 5 |
| | | W | | | |
| SW:A1AT_HUMAN | α-1-antitrypsin | K.YLGN#ATAIFFLPDEGK.L | 1.10 ± 0.11 | 1 | 10 |
| SW:A1AT_HUMAN | α-1-antitrypsin | R.QLAHQSN#STNIFF.S | 1.00 ± 0.01 | 1 | 0 |
| SW:AACT_HUMAN | α-1-antichymotrypsin | K.YTGN#ASALFILPDQDK.M | 1.05 ± 0.03 | 1 | 5 |
| SW:CO3_HUMAN | complement c3 | N.HMGN#VTFTIPANR.E | 0.91 ± 0.02 | 1 | 9 |
| SW:CO4_HUMAN | complement c4 | R.FSDGLESN#SSTQFEVK.K | 0.93 ± 0.07 | 1 | 7 |
| SW:HPT1_HUMAN | haptoglobin-1 | K.VVLHPN#YSQVDIGLIK.L | 1.04 ± 0.03 | 1 | 4 |
| SW:HPT1_HUMAN | haptoglobin-1 | K.NLFLN#HSEN#ATAK.D | 1.29 ± 0.33 | 1 | 29 |
| SW:IC1_HUMAN | plasma protease c1 inhibitor | R.VLSN#NSDANLELNTWV | 0.90 ± 0.03 | 1 | 10 |
| | | AK.N | | | |
| SW:ITH1_HUMAN | inter-α-trypsin inhibitor heavy | H.FFAPQN#LTNMNK.N | 0.96 ± 0.01 | 1 | 4 |
| | chain h1 | | | | |
| SW:ITH2_HUMAN | inter-α-trypsin inhibitor heavy | K.GAFISN#FSMTVDGK.T | 1.08 ± 0.12 | 1 | 8 |
| | chain h2 | | | | |
| SW:ITH4_HUMAN | inter-α-trypsin inhibitor heavy | N.QLVDALTTWQN#K.T | 1.01 ± 0.13 | 1 | 1 |
| | chain h4 | | | | |
| SW:KAIN_HUMAN | Kallistatin | K.FLN#DTMAVYEAK.L | 1.24 ± 0.30 | 1 | 24 |
| SW:KAL_HUMAN | plasma kallikrein | R.IYSGILN#LSDITK.D | 1.06 ± 0.08 | 1 | 6 |
| SW:KNG_HUMAN | Kininogen | K.LNAENN#ATFYFK.I | 0.94 ± 0.10 | 1 | 6 |

a: Gene name is from human NCBI protein database (www.ncbi.nlm.nih.gov).
b: The consensus motif for N-linked glycosylation is highlighted and the asparagine residues to which carbohydate linked are N#.

These results indicate that the method selectively isolates and quantifies N-linked glycopeptides from mixtures of glycoproteins.

### EXAMPLE VII

### Identification of N-linked Glycosylation Sites and Consensus Motif for N-linked Glycosylation

This example describes the identification of asparagine residues that are occupied by N-linked carbohydrates in the native protein and determination of consensus motif from the alignment of identified N-linked glycosylation sites.

Glycoproteins were conjugated to hydrazide resin and released from the solid support by PNGase F as described in Example I. PNGase F catalyzed cleavage of oligosaccharides from glycoproteins deaminates the linker asparagine to aspartic acid causing a mass shift of one mass unit. The single mass unit differences between asparagine and aspartic acid were detected by mass spectrometers and identify the asparagine residues to which the oligosaccharides were attached.

The one mass unit difference caused by conversion of asparagine to aspartic acid after cleavage of oligosaccharides from glycoproteins was specified in Sequest search parameter during database search of the MS/MS spectra. The acquired MS/MS spectra were searched against the human protein database from NCBI. For MS/MS spectra acquired by MALDI QqTOF (MDS SCIEX; Concord, Ontario CA), the mass window for the singly-charged ion of each peptide being searched was given a tolerance of 0.08 Da between the measured monoisotopic mass and the calculated monoisotopic mass, and the b, y, and z ion series of the database peptides were included in the Sequest analysis. For MS/MS spectra acquired by a Finnigan LCQ ion trap mass spectrometer, the mass window for each peptide being searched was given a tolerance of 3 Da between the measured average mass and the calculated average mass, and the b and y ion series were included in the Sequest analysis. The sequence database was set to expect the following possible modifications to certain residues: carboxymethylated cysteines, oxidized methionines and an enzyme catalyzed conversion of Asn to Asp at the site of carbohydrate attachment. There were no other constraints included in the Sequest search.

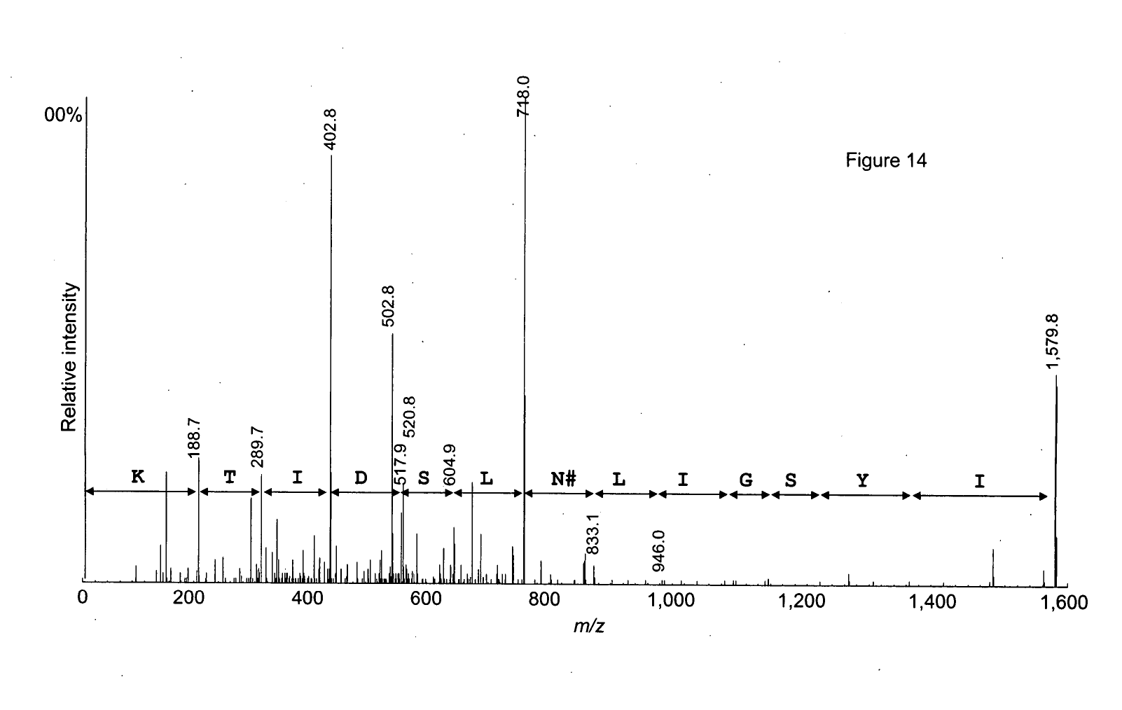

The precursor ion with m/z=1579.74 identified in Example VI was further analyzed by MS/MS and sequence database searching of the resulting spectrum, and it was identified with peptide sequence IYSGILN#LSDITK from human plasma kallikrein, a serum protease (FIG. 14). N# indicates the modified asparagine in the peptide sequence. The series of y ions from this peptide confirmed the match and indicates that the single mass unit difference between asparagine and aspartic acid can be easily detected by MALDI QqTOF mass spectrometry, thus confirming the precise glycosylation site within the peptide as N7.

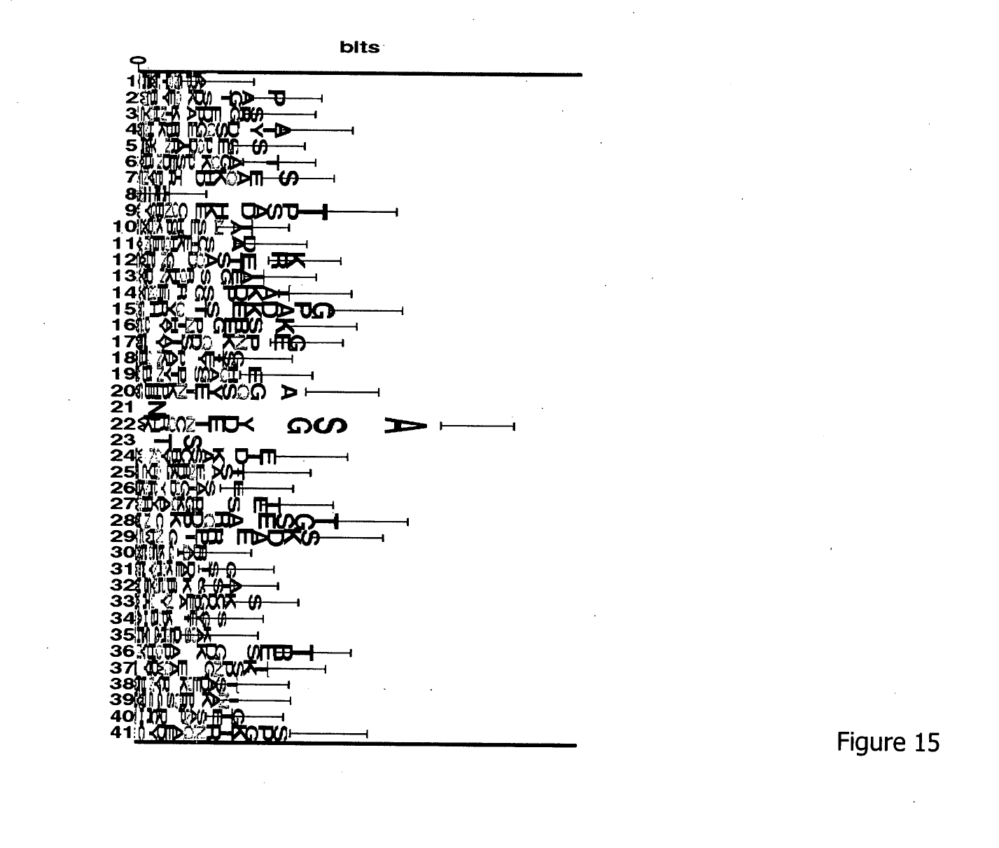

The peptides identified with N to D conversion were aligned using Sequence Logos (Schneider and Stephens, Nucleic Acids Res. 18:6097-6100 (1990)). FIG. 15 shows the patterns of aligned sequences. For each position in the aligned sequence, the height of each letter is proportional to its frequency, and the most common one is on top. As expected, there was high preference of N at position 21 in FIG. 15 (removed to show the detail of other positions). The preference of N was followed by S or T at position 23 (removed to show residues in other positions). This is a known consensus N-linked glycosylation motif. In addition, the preference of L, V, A, S, G at positions 9, 15, 20, 22, 24, 28, 29 was identified.

The identified glycopeptides were used to build a glycopeptide database. When searching a human database for potential N-linked glycosylation motifs with the previously defined NXS/T sequence, sixty percent of human proteins contain the consensus N-linked glycosylation motif. The alignment of identified N-linked glycopeptides by the glycopeptide capture method described here refined and extended the consensus N-linked glycosylation motif. The refined motif is used to generate an algorithm to search the entire database for possible N-linked glycosylation sites. This increases the database searching constraints and reduces the propensity of false identifications. Protein topology of known proteins or predicted protein topology from prediction programs such as PSORT II can be used to further increase the confidence of the predicted N-linked gycosylation motif since it is known that N-linked glycosylation occurs on extracellular domains and on the protein surface.

The increased prediction power for N-linked glycosylation sites can be used to search the candidate genes specific to ovarian cancer from microarray data of normal and ovarian cancer samples. The predicted N-linked glycosylation peptides are synthesized with the incorporation of stable isotope amino acids. 500 fmole of synthetic peptides are mixed with peptides purified from normal and ovarian cancer serum using the glycopeptide capture method described in Example I. The relative abundance of candidate peptides in normal and cancer patients are quantified with high accuracy and sensitivity. Since the peptide mass and MS/MS spectra of each synthetic peptide are known, the mass spectrometer can be set to run in single reaction monitor mode (SRM) with increased sensitivity and accuracy of quantification.

This example describes an exemplary method to identify glycosylation sites.

### EXAMPLE VIII

### Quantitative Profiling of Glycoproteins in Extracellular Matrix of Prostate Cancer

Prostate cancer is the most common cancer in men in the Western world, and the second leading cause of cancer mortality. The prostate is remarkably prone to developing cancer, and because little is known about the cause, no preventive measures can be formulated. With the use of prostate-specific antigen (PSA)-based screening, 80% of prostate cancer can be detected at a stage where it can be treated by local therapies. However, the rate of treatment failure as indicated by rising PSA levels can range from 10% to 40%. Apparently the escape of cancer cells from the prostate is an early event, and many patients test positive for these cells in their blood and bone marrow. A challenge in the diagnosis and treatment of prostate cancer is to develop better markers for cancer diagnosis to detect the disease at an early, more curable stage; to molecularly define prostate cancer progression for more accurate prognosis; and to identify cancer cell surface specific antigens as therapeutic targets.

Tumor and benign tissue samples from the peripheral zone of the prostate of the same patients were handled under sterile conditions. Tissue specimens were minced and digested with collagenase in RPMI-1640 medium supplemented with 10⁻⁸ M dihydrotestosterone (Liu et al., Prostate 40:192-199 (1999)). The digestion medium was saved, and glycoproteins were isolated as described in Example I.

The extracellular matrix protein species from patient-matched normal and cancer samples were processed by the glycopeptide capture method as described in Example I. The peptides released from the hydrazide resin were resuspended in 20 μl of 0.4% acetic acid. A 5 μl aliquot of sample was analyzed by μLC-MS/MS analysis, and the CID spectra were searched against the Human NCI database using Sequest.

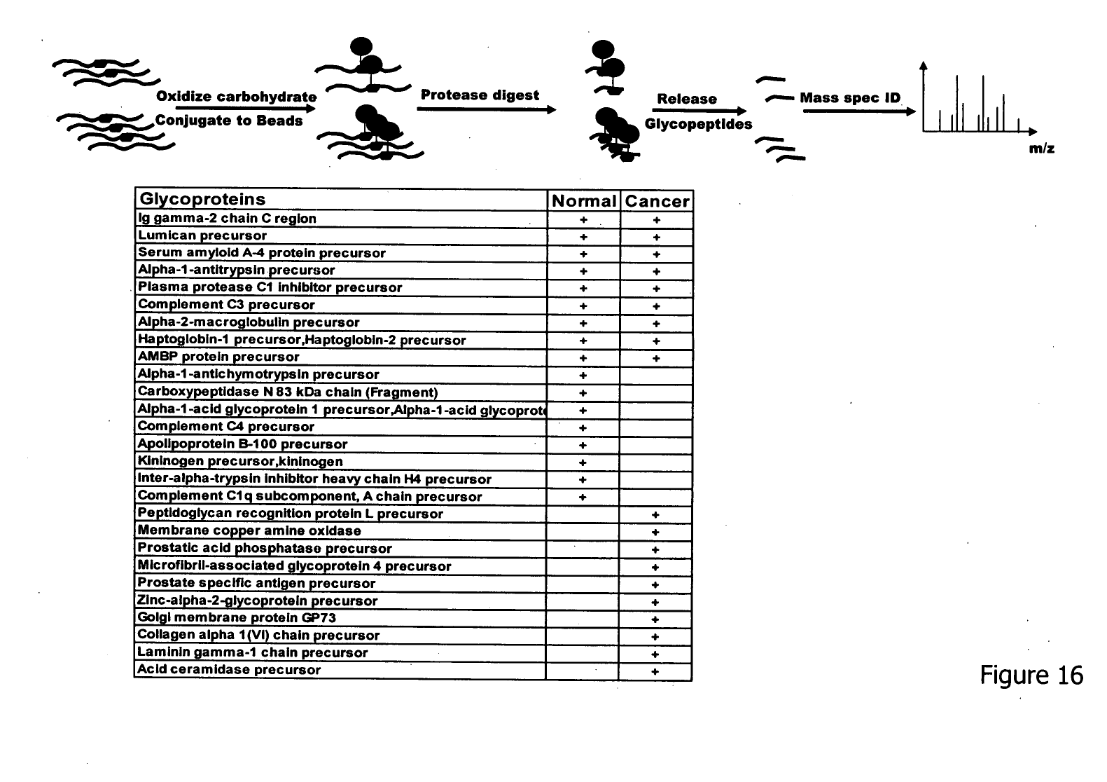

FIG. 16 shows the proteins identified from normal and cancer tissues. Two cancer specific proteins, prostate-specific antigen (PSA) and prostatic acid phosphatase (PAP), were readily detected in cancer tissues.

The formerly N-linked glycosylated peptides are labeled with light and heavy succinic anhydride as described in Example I, and peptides from normal (labeled with light succinic anhydride) and cancer (labeled with heavy succinic anhydride) samples are combined and analyzed by LC-MS/IMS. The CID spectra are searched against a human database, and the identified proteins are quantified using stable isotope quantification software tools such as ASASratio, and Express (Han et al., Nat. Biotechnol. 19:946-951 (2001)).

Since the concentration of specific proteins at the cancer tissue is much higher than that in blood serum, the cancer specific surface proteins are easily detected. The identified proteins can serve as cancer cell surface specific therapeutic targets. To determine the existence of cancer specific proteins in serum of prostate cancer patients, synthetic peptides are mixed with glycopeptides isolated from serum and analyzed by mass spectrometry. SRM mode analysis is used in the analysis and it increases the specificity and sensitivity of detecting the peptides in patient serum for early detection markers.

This example describes the identification of markers from cancer samples as potential diagnostic markers and/or therapeutic targets.

### EXAMPLE IX

### Quantitative Profiling of Glycopeptides and Identification of Biomarkers from Mice with Skin Cancer

This example describes identification of biomarkers associated with skin cancer.

Mass spectrometry has recently been used as a platform for protein-based biomarker profiling (Petricoin et al. Lancet 359:572-577 (2002)). It has been shown that pathological changes of tissues and organs are reflected in serum protein changes while blood circulates in the body. The reduced sample complexity and enriched biological information from the glycopeptide capture method provides advantages for the systematic investigation of serum protein expression patterns of thousands of proteins in serum.

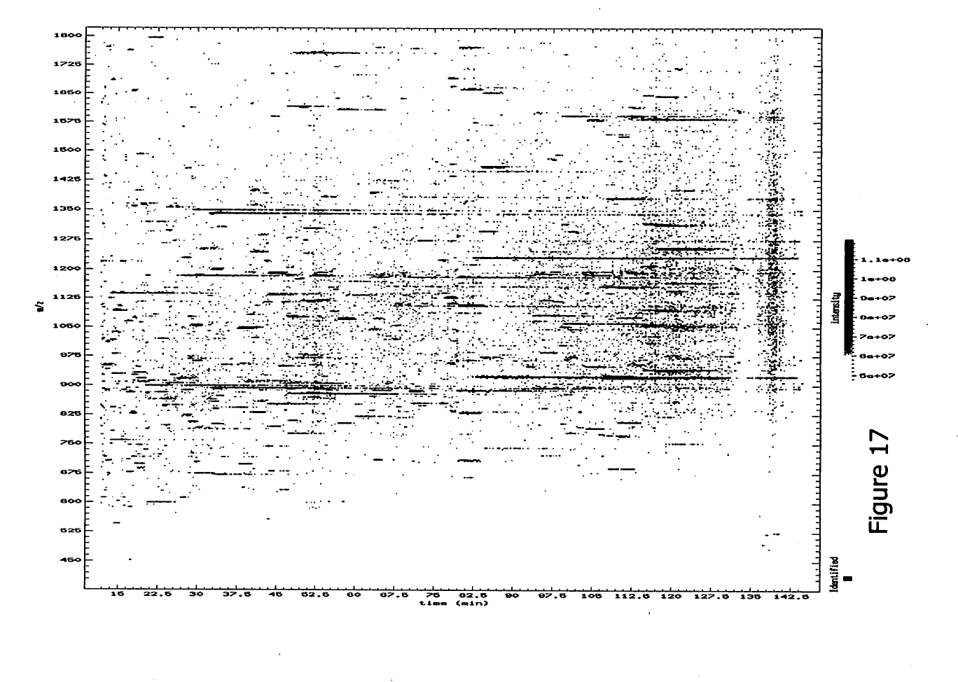

Several advantages of using the glycopeptide capture method and peptide mass to identify serum biomarkers are as follows. (1) It is fast and obviates the need for extensive separation methods. Because of the top down mode of operation of tandem mass spectometry in the time available during a LC-MS/MS experiment, only a fraction of the peptides present is selected for CID to identify the peptide sequence. Consequently, if peptides are detected by their mass only, in the time period of a LC-MS/MS experiment a significantly higher number of peptides can be detected than sequenced. This is illustrated in FIG. 17. The total number of peptides present in a single LC-MS/MS run is shown, and identified peptides are shown by the red dots. It was consistently found that less than 10% of total peptides were identified for complex biological samples. (2) The glycopeptide capture method simplified the total peptides present in serum after protease digestion and removed the heterogeneity of peptides caused by different oligosaccharides modifications and break down during MS analysis. (3) The majority of proteins and peptides in biological samples were unchanged in different states of the samples. Analyzing the relative abundance of all the peptides present in LC-MS, the peptides that change in abundance can be identified and the CID analysis focused on the differentially expressed proteins for identification.

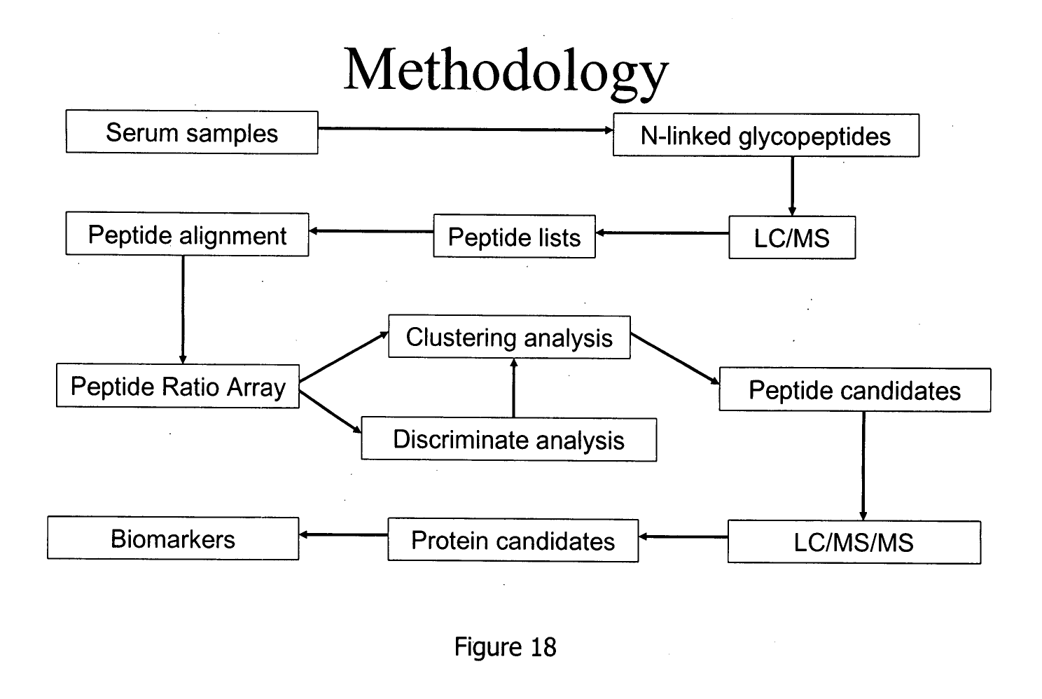

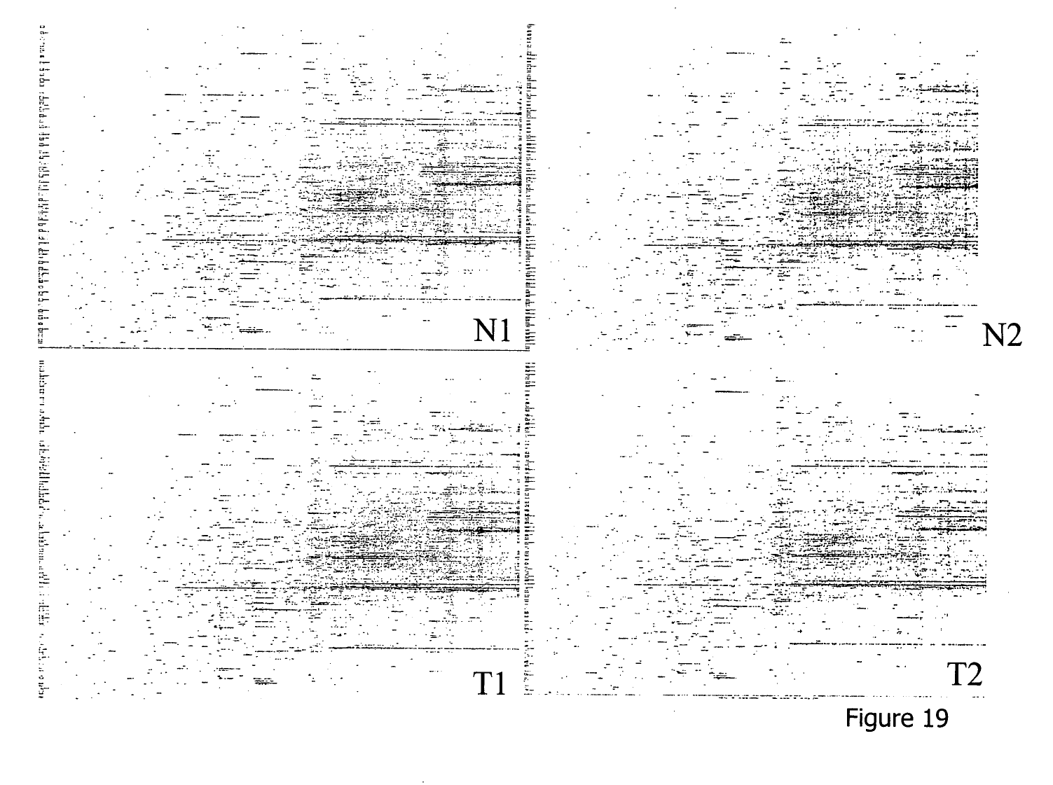

The strategy used to identify the biomarkers in serum is shown schematically in FIG. 18. Glycopeptides from 100 μl of serum from 10 normal and 3 diseased mice were purified as described in Example I. The peptides were resuspended in 30 μl 0.4% acetic acid, and 5 il of samples were analyzed by LC-MS/MS. FIG. 19 shows the signal intensity of peptides during the elution of the LC-MS/MS run. N1 and N2 were from normal mice, and T1 and T2 were glycopeptides from mice serum with skin cancer. Reproducible patterns of peptides from individual mice were observed during the LC-MS/MS runs.

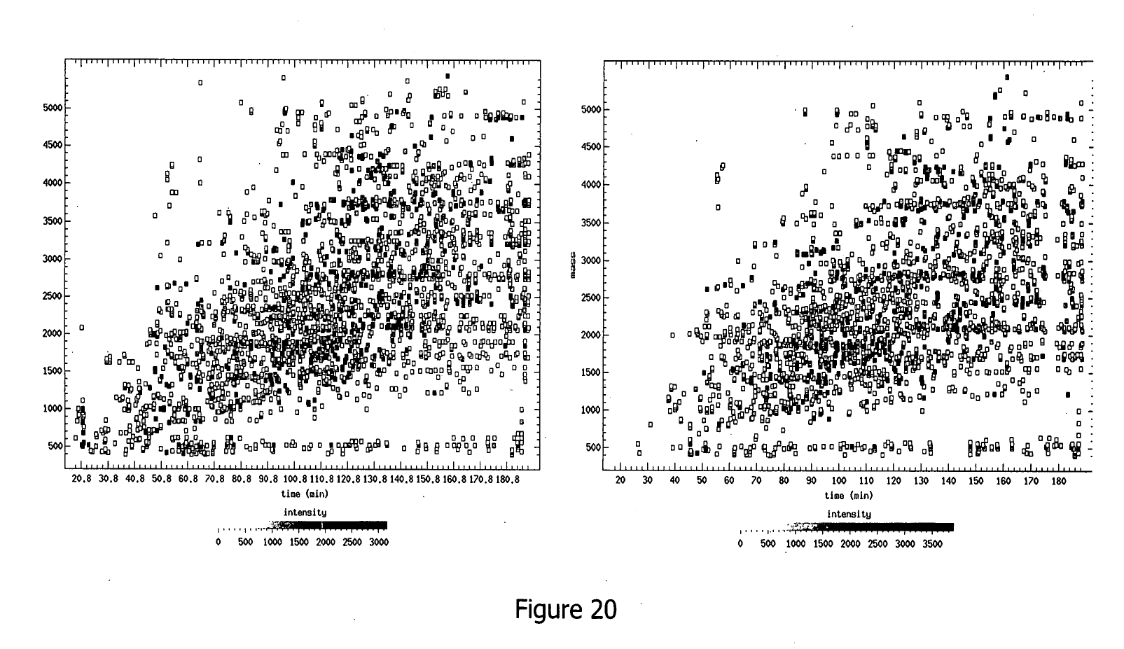

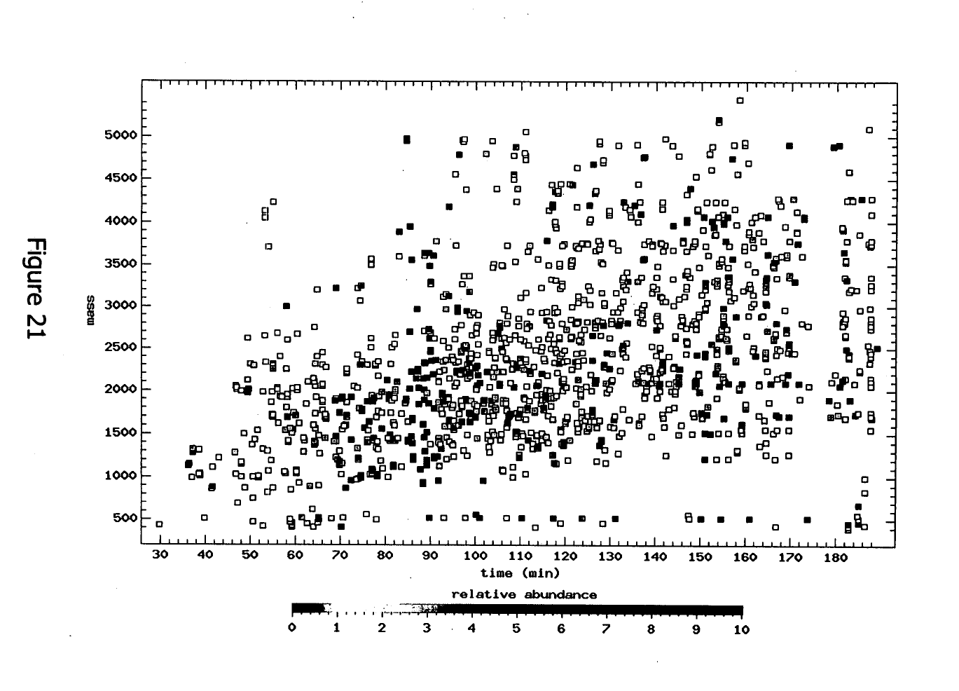

Peptide peaks from different charge states in the entire run were deconvoluted to signal charged peptides. FIG. 20 shows the deconvoluted peptides intensity during different elution time from normal and skin cancer mice. About 3000 peptides were consistently observed in different samples. The peptides were then aligned by elution time using in-house developed software, and normalized to background to reduce the variation of different runs. The relative peptide intensity of cancer mouse to normal mouse was calculated and shown in FIG. 21.

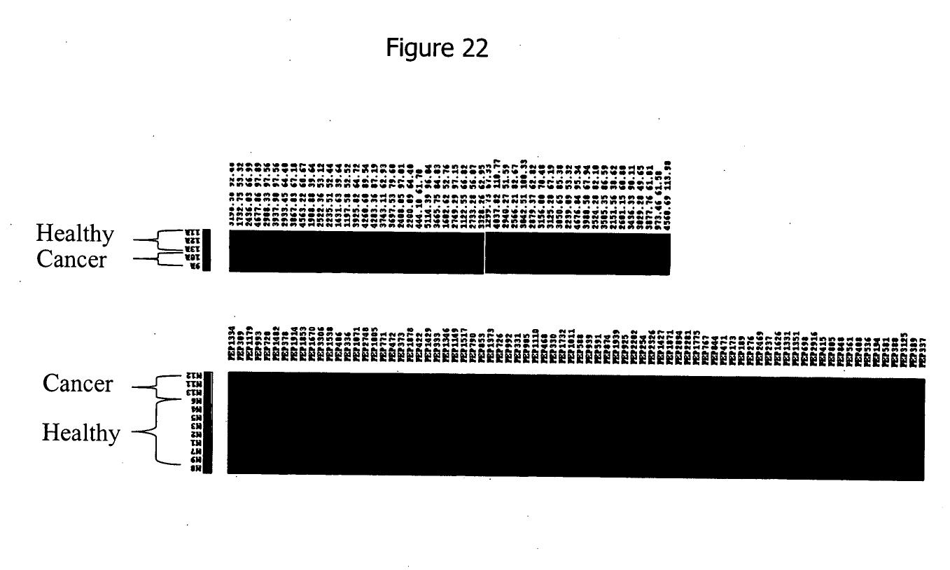

To facilitate the quantification, an equal amount of peptides from all 13 mice was mixed and analyzed by mass spectrometry as control. The relative intensity of each peptide to the control after alignment was obtained. The relative peptide intensities from all 13 mice from two different experiments was analyzed by unsupervised hierarchical clustering ((Eisen et al., Proc. Natl. Acad. Sci. USA 95:14863-14868 (1998)). No predefined reference vectors and prior knowledge of normal or cancer were used. In this clustering analysis, relationships among peptides were represented by a tree whose branch lengths reflect the degree of similarity between the objects. As shown in FIG. 22, all the cancer mice were found clustered together (indicated as 11A, 12A, 13A in experiment one, and M11, M12, M13 in experiment two). The peptide intensity shown in red indicates that the peptide abundance is lower than the corresponding peptide intensity in the common control, and the peptide intensity shown in green indicates a higher abundance of the peptide compared to the common control of the mixture.

This example shows that peptides isolated by the glycocapture method contain markers for cancer. The analysis of formerly N-linked glycopeptides using peptides mass and retention time increases the information of peptides during the mass spectrometry analysis. This approach is capable of distinguishing the difference between normal mice and mice with cancer and identifying cancer markers from serum.

### EXAMPLE X

### Quantitative Profiling of Glycopeptides from Human Serum Samples Obtained Before and After Overnight Fasting

This example describes the quantitative profiling and clustering analysis of glycopeptides from serum samples of three individuals before and after overnight fasting.

Glycopeptides from 100 μl of serum from three persons before and after overnight fasting were purified as described in Example I. The peptides were resuspended in 30 μl 0.4% acetic acid, and a control sample was made by mixing an equal amount (1 μl) of every glycopeptide from all 6 samples. A 5 μl aliquot of samples was analyzed by LC-MS/MS. The peptide peaks were deconvoluted to single charged peptides. After alignment and normalization of different runs, the relative intensity of each peptide to the common control sample was determined.

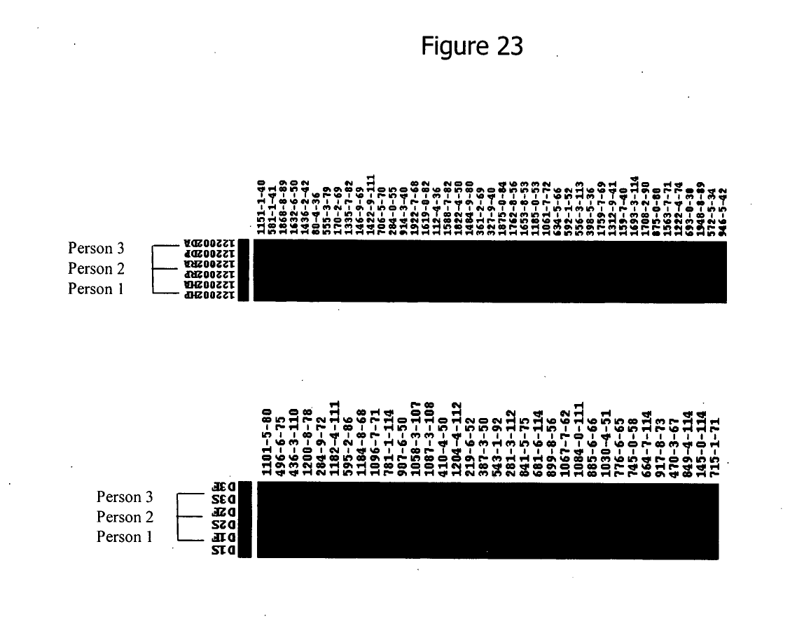

The relative peptide intensities from three individuals before and after overnight fasting were determined from each experiment and were analyzed by unsupervised hierarchical clustering without prior knowledge of any specificity and conditions of the individual samples ((Eisen et al., Proc. Natl. Acad. Sci. USA 95:14863-14868 (1998)). In this clustering analysis, relationships among peptide were represented by a tree whose branch lengths reflect the degree of similarity between the objects. As shown in FIG. 23, it was found that, in both experiments, serum samples from each individual before and after breakfast clustered together (indicated by person 1-3) in both experiments. The color coding is similar to that shown in FIG. 22.

These results show that peptides isolated by the glycocapture method from serum samples of each individual before and after overnight fasting are most closely related. The analysis of formerly N-linked glycopeptides using peptide mass and retention time increases the information on the peptides during the mass spectrometry analysis. This approach is capable of automatically distinguishing the most significant differences between the samples. This shows that glycopeptides from individual serum samples contain the characterstic features that can be used to assess the physiological state of an individual.

### EXAMPLE XI

### Determination of Glycosylation Occupancy from Serum Samples Obtained from Healthy Individuals or Patients with Type I Congenital Disorders of Glycosylation (CDG)

This example describes glycopeptide profiling of individuals with disorders of glycosylation.

Quantitative analysis of N-linked glycosylation is capable of determining the relative N-linked glycosylation in different proteomes. The cysteine tagging method can be used to determine the relative protein changes in different proteomes (Gygi et al., Nat. Biotechnol. 17:994-999 (1999)). By combining quantitative analysis of N-linked glycosylation with cysteine tagging, the occupancy of individual N-linked glycosylation sites and changes thereof can also be determined. This is of particular interest in studies in which changes of glycosylation occupancy are suspected, as exemplified by patients with Type I Congenital Disorders of glycosylation (CDG), in which the pathway of N-linked glycosylation is deficient (Aebi and Hennet Trends Cell Biol. 11:136-141 (2001)). In addition, changes in the extent of glycosylation and the carbohydrate structure of proteins on the cell surface and in body fluids have been shown to correlate with cancer and other disease states, highlighting the clinical importance of this modification as an indicator or effector of pathologic mechanisms (Spiro, Glycobiology 12:43R-56R (2002); Freeze Glycobiology 11:129R-143R (2001); Durand and Seta, Clin. Chem. 46:795-805 (2000)).

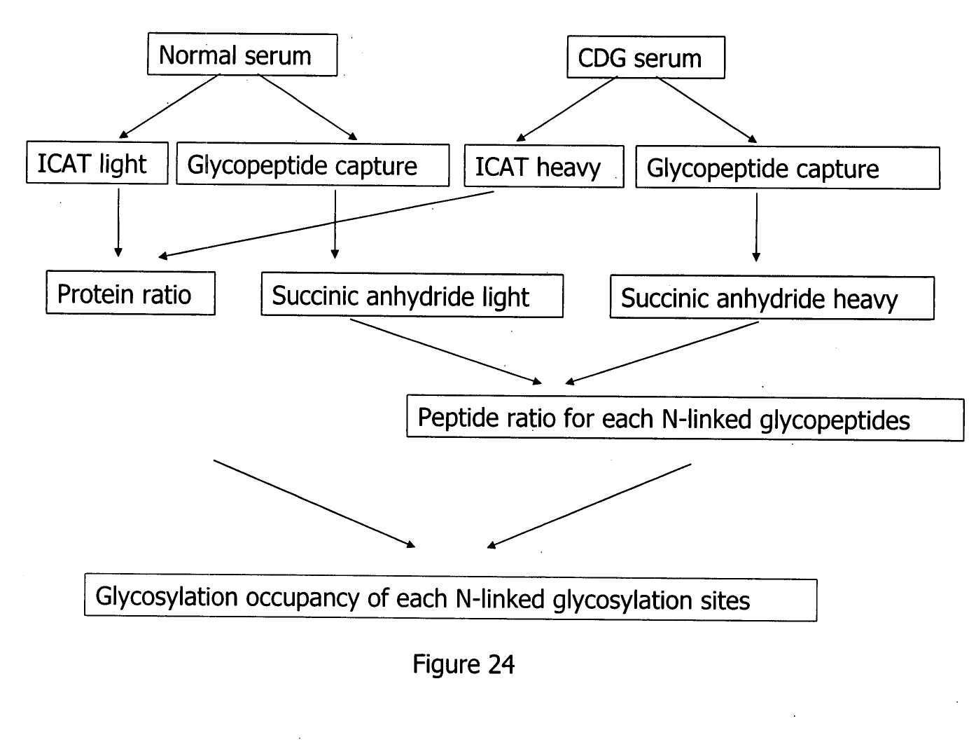

The glycosylation occupancy study of serum from CDG patients is described in FIG. 24. The ratio of total serum protein level of an individual was quantified using the ICAT reagent, and the ratio of N-linked glycopeptides of the individual is determined by glycopeptide capture followed by N-terminal isotopic labeling. The glycosylation occupancy is determined by the ratio of each N-linked glycopeptides divided by total protein ratio of the proteins.

To determine the relative ratio of total protein, the ICAT reagent was used to label the protein. Seven samples containing 0.5 mg of serum proteins from normal person #1 was labeled with the ICAT light reagent, and 0.5 mg of serum proteins from normal person #1, normal person #2, CDG 1a patient #1, CDG 1g patient #2, CDG 1b patient #1, CDG 1b patient #2, and CDG 1b patient #3 were labeled with ICAT heavy reagent. The ICAT reagent was purchased from Applied Biosystems, and labeling was performed following the manufactory's instruction.

Briefly, serum proteins (0.5 mg, 6.25 μl) were added to 0.5 ml of ICAT labeling buffer (6M urea, 0.05% SDS, 200 mM Tris, 5 mM EDTA, pH 8.3). The samples were reduced by adding 8 mM tris-carboxyethylphosphine (TCEP) and incubating at 37° C. for 45 minutes. Five fold excess of light and heavy ICAT reagents was added, and labeling was performed in the dark at 37° C. for 2 hours. The seven samples labeled with heavy ICAT reagent were mixed with one of seven normal samples labeled with light ICAT reagent. The seven mixed samples were diluted ten fold, and 5 μg of trypsin was added and incubated at 37° C. overnight. The ICAT labeled tryptic peptides were purified by avidin affinity chromatography using a Vision chromatography workstation from Applied Biosystems (Foster City, Calif.). The peptides were resuspended in 20 μl of 0.4% acetic acid, and 5 μl of peptides were analyzed by Finnigan LCQ ion trap mass spectrometer (Finnigan, San Jose, Calif.). The CID spectra were searched against the human NCI database using Sequest. A suite of software tools developed at the Institute for Systems Biology were used to analyze protein identification and relative expression ratio, including probability analysis of peptide and protein identification, and expression ratio of each protein (Eng et al., J. Am. Soc. Mass Spectrom. 5:976-989 (1994); Han et al., Nat. Biotechnol. 19:946-951 (2001); Keller et al., Anal. Chem. 74:5383-5392 (2002)). The protein ratio from normal person #1 to normal person #1 is shown in Table 6. The ratio agreed well with the expected 1:1 ratio.

TABLE 6
Protein expression ratio determined by ICAT labeling

| Protein name | ratio of protein expression |
|---|---|
| GP:A00279_1 | 0.803 +− 0.268 |
| GP:AB064062_1 | 0.826 +− 0.140 |
| GP:AB064121_1 | 0.975 +− 0.091 |
| GP:AJ390244_1 | 1.464 +− 0.269 |
| PIR2:A37927 | 0.795 +− 0.268 |
| SW:A2HS_HUMAN | 0.847 +− 0.224 |
| SW:A2MG_HUMAN | 0.967 +− 0.138 |
| SW:ALBU_HUMAN | 1.166 +− 0.036 |
| SW:ALC1_HUMAN | 1.352 +− 0.115 |
| SW:ALC2_HUMAN | 1.327 +− 0.539 |
| SW:AMBP_HUMAN | 1.208 +− 0.292 |
| SW:APA2_HUMAN | 1.047 +− 0.178 |
| SW:APOH_HUMAN | 0.991 +− 0.176 |
| SW:CFAB_HUMAN | 1.388 +− 0.757 |
| SW:CFAH_HUMAN | 1.043 +− 0.111 |
| SW:CO3_HUMAN | 0.896 +− 0.063 |
| SW:FIBB_HUMAN | 1.093 +− 0.218 |
| SW:FIBG_HUMAN | 0.949 +− 0.184 |
| SW:FINC_HUMAN | 1.391 +− 0.126 |
| SW:GC1_HUMAN | 1.195 +− 0.058 |
| SW:GC2_HUMAN | 1.047 +− 0.249 |
| SW:GC4_HUMAN | 1.195 +− 0.425 |
| SW:HEMO_HUMAN | 1.057 +− 0.086 |
| SW:HPT1_HUMAN | 1.152 +− 0.091 |
| SW:HPTR_HUMAN | 1.377 +− 0.206 |
| SW:ITH2_HUMAN | 1.330 +− 0.314 |
| SW:KAC_HUMAN | 1.072 +− 0.146 |
| SW:LAC_HUMAN | 1.063 +− 0.592 |
| SW:MUCB_HUMAN | 1.191 +− 0.536 |
| SW:MUC_HUMAN | 0.986 +− 0.221 |
| SW:TRFE_HUMAN | 1.183 +− 0.061 |
| SW:VTDB_HUMAN | 1.394 +− 0.204 |

To determine the relative glycosylation ratio of each N-linked glycosylation site, seven aliquots of 1 mg (12.5 μl) from normal person #1, and 1 mg of serum from normal person #1, normal person #2, CDG 1a patient #1, CDG 1g patient #2, CDG 1b patient #1, CDG 1b patient #2, and CDG 1b patient #3 were subjected to the glycopeptide capture method as described in Example I. Glycopeptides from the seven samples from normal person #1 were labeled with light succinic anhydride, and the other samples were labeled with heavy succinic anhydride while the glycopeptides were still attached to solid beads. The paired normal and seven individuals are mixed, and formerly N-linked glycosylated peptides are released. The peptides were resuspended in 20 μl of 0.4% acetic acid and 5 μl of peptides were analyzed by Finnigan LCQ ion trap mass spectrometer (Finnigan, San Jose, Calif.). The CID spectra were searched against the human NCI database using Sequest, a suite of software tools developed in Institute for Systems Biology were used to analyze the peptide and protein probability and protein expression ratio using ASAPratio. The ratio of glycosylated peptides is divided by the total protein ratio, and the glycosylation occupancy is determined for each N-linked glycosylation sites.

### EXAMPLE XII

### Determination of the Level of Glycosylation from Diabetic Obese Mouse Serum

This example describes the determination of glycosylation in a model of diabetes.

Nonenzymatic glycation in diabetes results from the reaction between glucose and primary amino groups on proteins to form glycated residues. The glycated proteins and the later-developing advanced glycation end-products have been mechanistically linked to the pathogenesis of diabetic nephropathy. Glycated albumin has been causally linked to the pathobiology of diabetic renal disease (Cohen and Ziyadeh, J. Am. Soc. Nephrol. 7:183-190 (1996)).

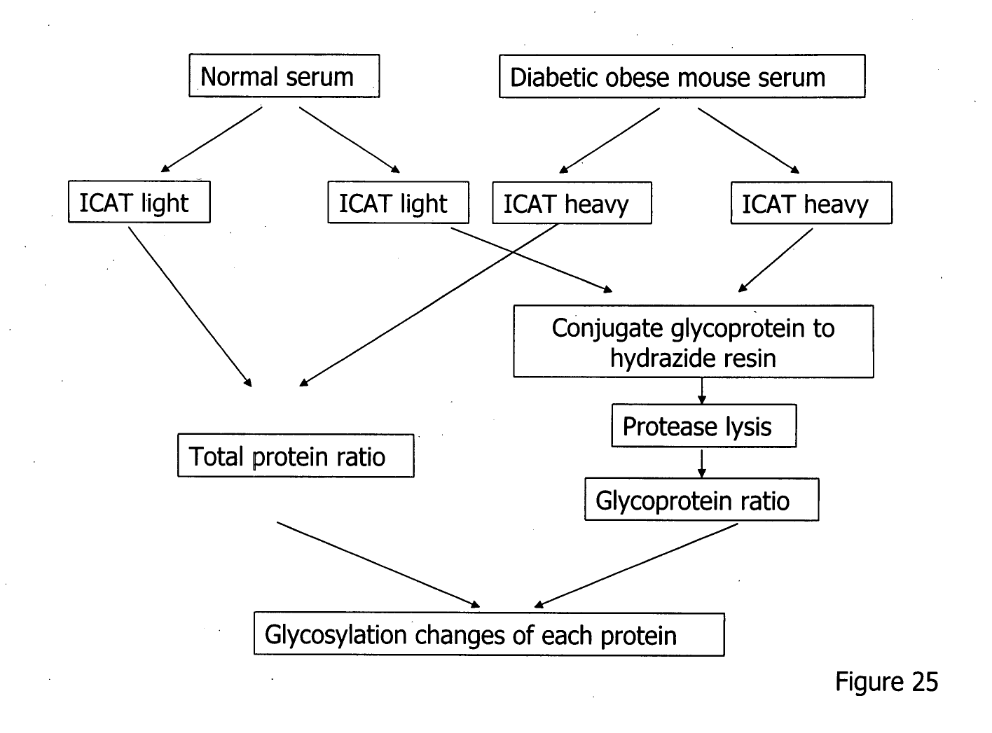

Other proteins in serum may be also responsible for the development of diabetic complications. Samples are analyzed for changes in carbohydrate modified serum proteins. Serum from wild type liter mates and diabetic obese mice from BTBR mouse strain are labeled with light and heavy ICAT reagent as shown in FIG. 25. The labeled serum samples are divided into two equal fractions, and paired light and heavy serum from normal and diabetic obese mouse samples are mixed. One mixture is used to determine the total serum protein ratio using the ICAT measurement. The second mixture is conjugated to a solid support using hydrazide chemistry. The cysteine containing peptides from glycoproteins are released by trypsin and isolated by avidin chromatography column using the Vision chromatography workstation (ABI). The relative abundance of glycoproteins between normal and diabetic mice is determined. After normalization to the total protein in serum, the changes of glycosylation are determined.

This experiment shows that the glycopeptide capture method can be used to analyze enzymatically glycosylated proteins as well as non-enzymatic lysine glycation of proteins. The level of non-enzymatic glycation increases in certain diseases caused by diabetes due to the high glucose levels in the patient's blood serum.

### EXAMPLE XIII

### Quantification of N-linked Glycopeptides Using Heavy Isotope Labeled Synthetic Peptide Standards

This example describes quantification using labeled synthetic peptide standards.

Table 7 shows several synthetic peptides (SEQ ID NOS: 198-209) identified from human serum, as described in Example II. The peptides were synthesized using standard solid phase synthesis chemistry with the carbon 13 amino acid incorporated in the valine residues at the underlined position. The glycosylated asparagines were also changed to aspartic acid. 500 fmol of each peptide was mixed and run separately on LC-MS/MS analysis to determine the retention time and CID spectra. The same amount of peptides was mixed with human serum samples from three individuals to determine the relative amount of these glycopeptides in serum.

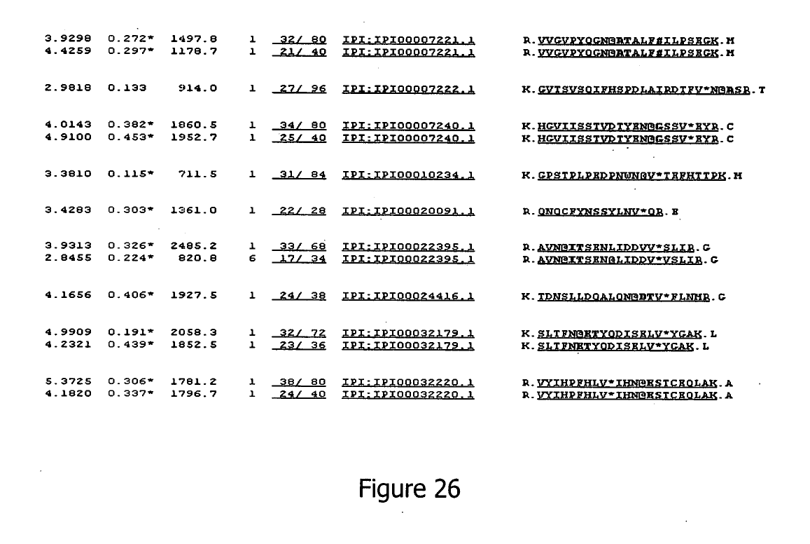

FIG. 26 shows the synthetic peptides identified by mass spectrometry.

TABLE 7
Synthetic heavy isotope labeled peptide standards.

| | |
|---|---|
| Protein Name | Peptide Sequence |

| | |
|---|---|
| Plasma protease C1 inhibitor precursor | GVTSVSQIFHSPDLAIRDTFVDASR |
| Angiotensinogen precursor [Contains: Angiotensin I] | VYIHPFHLVIHDESTCEQLAK |
| Pigment epithelium-derived factor precursor | VTQDLTLIEESLTSEFIHDIDR |
| Serum amyloid A-4 protein precursor | SRVYLQGLIDYYLFGDSSTVLEDSK |
| Complement component C9 precursor | AVDITSENLIDDVVSLIR |
| Biotinidase precursor | YQFNTNVVFSNDGTLVDR |
| Coagulation factor XIII B chain precursor | HGVIISSTVDTYEDGSSVEYR |
| Alpha-1-acid glycoprotein 2 precursor | QNQCFYDSSYLNVQR |
| Plasma serine protease inhibitor precursor | VVGVPYQGDATALFILPSEGK |
| Aminopeptidase N | GPSTPLPEDPNWDVTEFHTTPK |
| Antithrombin-III precursor | SLTFDETYQDISELVYGAK |
| ICOS ligand precursor | TDNSLLDQALQDDTVFLNMR |

The samples are analyzed for glycopeptides as described in Example I. These results show that a known amount of synthetic peptides can be used to determine the relative or absolute amount of the same glycopeptides in individual serum samples.

### EXAMPLE XIV

### dentification of O-linked Glycopeptides Using Enzymatic Cleavage

This example describes identification of O-linked glycopeptides.

Analogous strategies to those described herein for analysis of N-linked glycosylation sites can be used to also analyze O-glycosylated peptides. In fact, a protein sample, once immobilized on a solid support, can be subjected to sequential N-linked and O-linked glycosylation peptide release, thus further increasing the resolution of the method and the information contents of the data obtained by it. There is no enzyme comparable to PNGase F for removing intact O-linked sugars. To release O-linked oligosaccharides, monosaccharides are sequentially removed by using a panel of exoglycosidases until only the Galβ1,3GalNAc core remains attached to the serine or threonine residue. The core can then be released by O-glycosidase. Since not all O-linked oligosaccharides contain this core structure, a chemical method, such as β-elimination can be more general and effective for the release of the formerly O-linked glycosylated peptides.

After releasing N-linked glycopeptides, 100 μl of hydrazide resin was washed with 1 ml of 1.5 M NaCl twice, 1 ml of 100% methanol twice, and 1 ml of water twice. O-linked glycopeptides were cleaved by a set of enzymes (Calbiochem), including Endo-α-N-acetylgalactosaminidase, Neuraminidase, β1,4-Galactosidase, and β-N-Acetylglucosaminidase. The released peptides were dried and resuspended in 0.4% acetic acid for LC-MS/MS analysis.

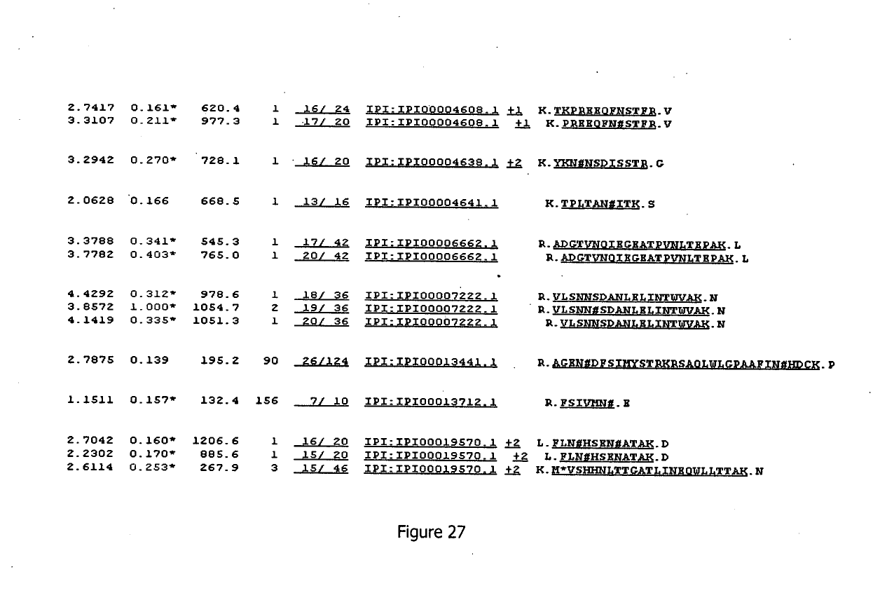

FIG. 27 shows the identified peptides from the series of enzymatic cleavages from hydrazide resin after N-linked glycopeptides were released. Unlike the N-linked glycosylation, in which PNGase F converts the glycosylated N to D after release of oligosaccharides, O-linked glycosylated serine or threonine remained unchanged. There are no known consensus motifs available for O-linked glycosylation. To date, the serine or threonine residues to which the O-linked oligosaccharides were attached have not been identified.

This example demonstrates that O-linked glycopeptides can also be identified.

### EXAMPLE XV

### Identification of Glycopeptides Isolated by Biotin Tagged Hydrazide

This example describes identification of glycopeptides isolated by biotin tagged hydrazide.

The same procedure described in Example I was also performed in solution phase using biotin tagged hydrazide (PIERCE) with some modifications. After proteins were oxidized and conjugated to biotin hydrazide, the proteins were denatured in 0.5% SDS and 8M urea in 0.4 M NH₄CO₃ for 30 minutes at room temperature. The samples were diluted 4 times with water, and trypsin was added at a final concentration of 1:100. The trypsin digest was performed overnight at room temperature. The glycopeptides conjugated to biotin hydrazide were purified by an avidin column using the Vision chromatography workstation. The glycopeptides were isolated with oligosaccharides still attached to the peptides. The peptides were dried and resuspended in 0.4% acetic acid and analyzed by mass spectrometry.

When high spray voltage was used in ESI-LC-MS/MS analysis (2.0 kv), the oligosaccharides were separated from peptide at the source. It resulted in the analysis of N-linked peptides and O-linked peptides by mass spectrometry. The identified N-linked glycopeptides are shown in FIG. 28, with the consensus NXT/S motif highlighted. The O-linked oligosaccharides were removed in the source with a loss of water. This left the formerly O-linked glycosylation Ser or Thr with 18 Dalton less than the unmodified Ser or Thr. This is represented in FIG. 29 at S or T without modification (shown in circles).

These results show that the glycopeptide capture method can also be performed via affinity reactive tags attached to the protein by solution chemistry. The glycopeptides isolated by this method can have oligosaccharide chains attached to the glycopeptides. Both N-linked and O-linked glycopeptides can be isolated and analyzed simultaneously.

### EXAMPLE XVI

### Automation of the Glycopeptide Capture Method Using a TECAN Workstation

This example describes adaptation of the glycopeptide analysis method to automation.

To improve the throughput and reproducibility of the method of glycopolypeptide analysis, an automated robotic workstation was designed to perform the sequence of reactions for glycopeptide isolation. The workstation is particularly useful for all applications requiring high sample throughput. The procedure described in Example I is tested in solid phase extraction format for automation in serum biomarker identification

A TECAN workstation was designed for the glycopeptide capture procedure. The workstation is used to automate sampling and analysis of glycopeptides. The workstation can be readily adapted to diagnostic applications, for example, the analysis of a large number of serum samples or other biological samples of diagnostic interest.

### EXAMPLE XVII

### Glycopeptide Capture Coupled with Mass Spectrometry for Glycoproteomics

This example describes shotgun glycopeptide-capture coupled with mass spectrometry for comprehensive glycoproteomics.

The materials were obtained and methods were carried out as described below. All glycoproteins were obtained from Sigma (St. Louis Mo.). MALDI matrix was from Agilent Technologies (Wilmington Del.). Bradford assay reagent, sodium periodate and hydrazide resin were obtained from Bio-Rad (Hercules Calif.). PNGase F was from New England Biolabs (Beverly Mass.). The cell culture reagents and media were from Invitrogen (Carlsbad Calif.). Tris(2-carboxyethyl)phosphine hydrochloride (TCEP) was from Pierce (Rockford IL), and trypsin was from Promega (Madison Wis.). RapiGest™ and C18 spin columns were from Waters (Milford Mass.). Zip-Tip was from Millipore (Bedford Mass.). All other chemicals were purchased from Fisher Scientific (Pittsburgh Pa.).

Cell culture and microsomal fraction extraction. IGROV-1/cp cisplatin-resistant ovarian-cancer cells were grown in RPMI 1640 medium (Invitrogen) containing 10% fetal bovine serum, 100 units/ml penicillin, and 100 units/ml streptomycin at 37° C. A crude microsomal fraction of IGROV-1/CP was prepared as described (Han et al., Nat. Biotechnol. 19:946-951 (2001); Stewart et al., Mol. Cell. Proteomics 5:433-443 (2006)). The microsomal pellet contained plasma membranes, Golgi apparatus, endoplasmic reticulum, mitochondria, lysosomes, and all other membrane-bound vesicles separated from soluble cytosol. The Bradford protein assay was used to quantify the concentration of the extracted proteins. About 0.5-0.8 mg of crude microsomal membrane proteins were used to proceed with the glycopeptide capture.

Tryptic digestion of samples. In the glycopeptide-capture approach, biological samples were subject to denaturation and trypsin digestion first. In a typical procedure, a biological sample was reconstituted in a denaturing buffer of 5 mM EDTA, 40 mM Tris, 10 mM TCEP, 0.5% RapiGest™ at pH 8.3, and heated at 100° C. for 10 min. After allowing the sample to cool to room temperature, urea was added to 8 M and the solution was incubated at 37° C. for 30 min. To prevent disulfide bond formation, cysteine residues were modified by alkylation with iodoacetamide. Iodoacetamide was added to the sample solution in at least a 6-fold molar excess over the free sulfhydryls in the sample. For an unknown protein mixture, an estimation of 6 cysteines per protein was used for calculating the molar concentration of sulfhydryls. A 30-min incubation in the dark, at room temperature, with end-over-end rotation was carried out for cysteine derivatization. The reaction was quenched by the addition of dithiothreitol (DTT) at half of the molar concentration of the iodoacetamide for 10 min. After iodoacetamide deactivation, the sample solution was diluted 10 fold with 40-mM Tris buffer, pH 8.3, and 1 mg trypsin was added into the sample solution per 20-50 mg of protein and the sample mixture was digested at 37° C. overnight. To avoid a large volume for trypsin digestion, the denatured sample was kept at 4-6 mg/ml. RapiGest™ was degraded by acidifying the trypsinized sample mixture to pH˜1 with HCl and incubation at 37° C. for 1 hr. The hydrophobic residues of RapiGest™ were precipitated out and removed from the sample by centrifugation; and the supernatant was passed over a C18 column to remove extra urea, DTT, and Tris. Tryptic peptides were eluted from the column with 80% acetonitrile in 0.1% TFA and dried in a SpeedVac® (Thermo Savant, Holbrook, N.Y., USA) concentrator.

Glycopeptide capture. Dried tryptic peptides were dissolved in a coupling buffer (100 mM sodium acetate, 150 mM NaCl, pH 5.5) at a concentration of 2 mg/100 μl buffer. The non-dissolved solids were removed by centrifugation, and the supernatant was ready for the following reactions. First, to oxidize the cis-diol groups of carbohydrates to aldehydes, sodium periodate at 10 mM final concentration was introduced into the peptide solution and the sample was incubated in the dark at room temperature for 30 minutes with end-over-end rotation. Second, sodium sulphite was added to 20 mM final concentration and incubated for 10 min to deactivate the excess oxidant in the peptide solution. The coupling reaction was initiated by introducing hydrazide resin into the quenched peptide solution at 20 mg/ml of resin, and extra coupling buffer was added to make a solid to liquid ratio of 1:5. The coupling reaction was performed at 37° C. overnight with end-over-end rotation.

After the coupling reaction, the resin was washed twice thoroughly and sequentially with deionized water, 1.5 N NaCl, methanol, acetonitrile; and was followed by a buffer exchange step to 100 mM NH₄HCO₃ (made fresh), pH˜8.0. Enzymatic cleavage of the N-linked peptides from the sugar moiety was carried out at 37° C. overnight by PNGase F at a concentration of 1 μl of PNGase F per 2-6 mg of crude proteins. The supernatant, containing the released de-glycosylated peptides, was collected by centrifugation and combined with the supernatant of an 80%-acetonitrile wash. The peptide solution was dried, reconstituted with 1% acetonitrile in 0.1% formic acid and subjected to mass spectrometry (MS) analysis.

Analysis of peptides by mass spectrometre. Peptide samples were analyzed by either a MALDI-TOF/TOF tandem mass spectrometer (ABI 4700 Proteomics Analyzer, Applied Biosystems, Foster City, Calif.) or a nanoLC-ESI-MS/MS using LTQ linear ion trap mass spectrometer (Thermo Finnigan, San Jose, Calif.). For MALDI-TOF/TOF analysis, the peptide sample was purified with a Ziptip (Millipore) and reconstituted with 0.4% acetic acid prior to analysis. A 1:1 dilution of peptide solution with MALDI matrix solution (Agilent Technologies) was used for MALDI spotting.

For LTQ mass spectrometry analysis, an in-house fabricated nanoelectrospray source and an HP 1100 solvent delivery system (Agilent, Palo Alto, Calif.) were coupled to LTQ. Samples were automatically delivered by a FAMOS autosampler (LC Packings, San Francisco, Calif.) to a 100 μm internal diameter fused silica capillary pre-column packed with 2 cm of 200 Å pore-size Magic C18AQ™ material (Michrom Bioresources, Auburn, Calif.) as described elsewhere (Yi et al., Rapid Commun. Mass Spectrom. 17:2093-2098(2003)).

The samples were washed with solvent A (5% acetonitrile in 0.1% formic acid) on the pre-column and then eluted with a gradient of 10-35% solvent B (100% acetonitrile) over 30 minutes to a 75 μm×10 cm fused silica capillary column packed with 100 Å pore-size Magic C18AQ™ material (Michrom) and then injected into the mass-spectrometer at a constant column-tip flow rate of ˜300 nL/min. Eluting peptides were analyzed by nanoLC-MS and data-dependent nanoLC-MS/MS acquisition, selecting the 3 most abundant precursor ions for MS/MS with a dynamic exclusion setting of 1 (Gygi et al., Mol. Cell. Biol. 19:1720-1730 (1999)).

Database search of mass spectra. Mass spectra were converted to mzXML format through in-house developed software, and the spectra have fewer than 6 ions with intensity less than 100 were discarded (Keller et al., Mol. Syst. Biol. 1:msb4100024-E1-msb4100024-E8 (2005); Pedrioli et al., Nat. Biotechnol. 22:1459-1466 (2004)). The converted mzXML files were searched against the appropriate databases (see below). The mass spectra derived from the five multiglycosylated proteins were searched against a customized database comprised of the protein sequences of 5 glycoproteins in addition with trypsin, keratins (a common contamination of sample preparation). 218 entries of human keratins were taken from NCI non-redundant protein database released on Dec. 13, 2005, (distributed on the Internet via anonymous FTP from ftp.ncifcrf.gov, under the auspices of the National Cancer Institute's Advanced Biomedical Computing Center), and the reversed protein sequences from a yeast protein databases with 7556 entries. The mass spectra of peptides from the ovarian-cancer cell line membrane fraction were searched against the human International Protein Index (IPI) database (IPI human v3.16 fasta with 62322 entries). SEQUEST™ (Thermo Finnigan) was used for database searches with search parameters containing the following modifications: carbamidomethylated cysteine (+57), oxidized methionines (+16) and the asparagine in the consensus sequence to aspartic acid modification after PNGase F deglycosylation (+1) (Carr et al., Anal. Biochem. 157:396-406(1986)). PeptideProphet™ and Protein Prophet with single tryptic end and N-glycosylation constraints were used to evaluate the quality of peptide and protein identification (Keller et al., Anal. Chem. 74:5383-5392 (2002)). Single tryptic end constrain was used to account for incomplete trypsin digestion due to different digestion efficiency by trypsin at putative tryptic sites (Keil, Specificity of proteolysis p 335, Springer-Verlag Berlin-Heidelberg-NewYork: (1992). The mass tolerance for precursor mass is ±3.0, and the mass tolerance for MS/MS is 0.5 (Eng, J. J. Am. Soc. Mass Spectrom. 5:976-989 (1994)).

The design of the glycopeptide capture approach. The complete workflow of the glycopeptide capture approach is elaborated in FIG. 30C. First, proteins were denatured and cleaved into peptides by trypsin digestion, then the cis-diol groups on the oligosaccharide chains were oxidized into aldehydes by sodium periodate for chemical coupling. After deactivating the excess periodate ions by sodium sulphite (Wu et al., Nucleic Acids Res. 24:3472-3473 (1996)), hydrazide resins were introduced into the same sample vessel directly to initiate capture. Immediately following the capture, the resin was thoroughly washed with stringent reagents to remove nonspecifically bound molecules. Finally, the captured N-linked glycopeptides were liberated from the solid support by PNGase F cleavage and subjected to tandem MS proteome analysis.

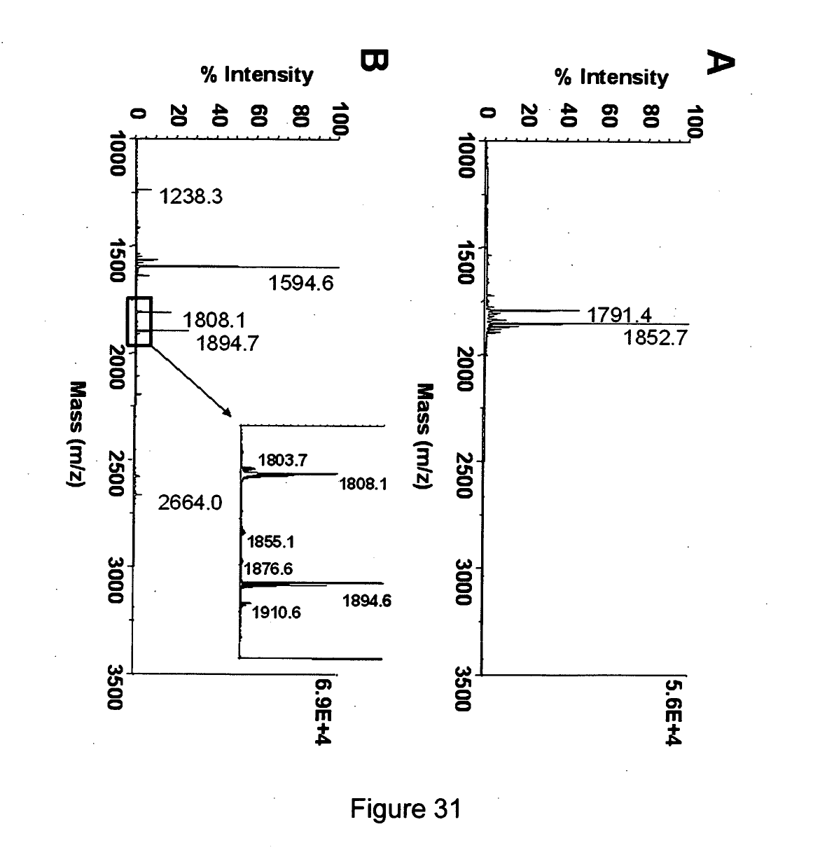

Application of the glycopeptide-capture approach to a mono-glycosylated protein. To validate the glycopeptide capture approach, a mono-N-glycosylated protein, chicken avidin, was first analyzed. FIG. 31A shows the MS spectrum of the enriched avidin deglycosylated glycopeptide collected by a MALDI-TOF/TOF mass spectrometer (ABI 4700 Proteomics Analyzer, Applied Biosystems). The two major peaks with m/z values differing by 61 were from the same peptide (K.WTNDLGSNMTIGAVNSR.G) as determined by MS/MS fragmentation. The mass of 1852 is contributed from the peptide with methionine oxidized to methionine sulfoxide (with a mass increase of 16 Da) (Zhang et al., Nat. Biotechnol. 21:660-666 (2003)), and the asparagine from the consensus sequence modified to aspartic acid (with a mass increase of 1 Da) after PNGase F deglycosylation (Carr et al., Anal. Biochem. 157:396-406 (1986)). The mass of 1791 is attributed from the same peptide as showed by the CID spectrum with a cleavage between the gamma carbon and the sulfur of the methionine side chain, which is most likely happened during the mass spectrometry direction. Jiang et al., J. Mass Spectrom. 31:1309-1310 (1996); Lapko et al., J. Mass Spectrom. 35:572-575 (2000); Reid et al., J. Am. Soc. Mass Spectrom. 16:1131-1150 (2005)). The successful identification of the formally glycosylated peptide from chicken avidin indicated that the capture strategy was effective, and that the sodium sulphite introduced to reduce periodate did not interfere with the capture procedure. To evaluate the capture efficiency, the non-captured avidin peptide mixture was analyzed by MALDI-TOF/TOF as well. Since glycosylated peptides give low and complex signals in MS spectra due to the heterogeneity of the glycan structure, we used PNGase F was used to remove all linked glycans prior to MS analysis and focused on the presence or absence of the deglycosylated glycopeptides from the non-captured fraction of chicken avidin. FIG. 31B shows the MS spectrum of the non-captured avidin peptides after pursuing the glycopeptide capture and PNGase F deglycosylation. The absence of glycopeptide signal indicates that the efficiency of the glycopeptide-capture strategy is highly efficient based on the MALDI-TOF/TOF analysis.

Application of the glycopeptide-capture strategy to a five-glycoprotein mixture. To further validate the glycopeptide-capture approach and to characterize the capture efficiency, the glycopeptide-capture strategy was applied to a protein mixture with five N-glycosylated proteins: invertase (yeast), α-1 antitrypsin (human), conalbumin (chicken), ribonuclease B (bovine) and ovalbumin (chicken) (all purchased from Sigma). Table 8 lists the representative N-linked glycopeptides captured and identified by the approach using a nanoLC-MS/MS analysis on an LTQ linear ion trap mass spectrometer. All of the proteins were identified with a protein prophet value of 1.0. Strikingly, all the previously identified glycosylation sites within the five glycoproteins were captured and identified, except for some sites from invertase (Table 8). For invertase that has 13 N-glycosylation sites, a total of 8 N-linked sites were identified (shown in Table 8).

TABLE 8

Results of LTQ analysis of N-glycopeptides isolated
from the five glycoprotein mixture.

| | | Reported | Detected |
|---|---|---|---|
| Protein name | Sequences of identified peptides^{a} | glycosylation site | glycosylation site |
| Invertase | S.MTNETSDRPLVHFTPNK.G | 13 | 8 |
| | K.NPVLAANSTQFR.D | | |
| | Y.FVGSFNGTHFEAFDNQSRV | | |
| | K.AEPILNISNAGPWSR.F | | |
| | R.FATNTTLTK.A | | |
| | L.VYAVNTTQTISK.S | | |
| | F.MTTGNALGSVNMTTGVDNLFYIDKFQVR.E | | |
| α1 Antitrypsin | R.QLAHQSNSTNIFFSPVSIATAFAMLSLGTK.A | 3 | 3 |
| | K.ADTHDEILEGLNFNLTEIPEAQIHEGFQELLR.T | 3 | 3 |
| | K.YLGNATAIFFLPDEGK.L | | |
| Conalbumin | G.WVIPMGLIHNR.T | 1 | 1 |
| Ribonuclease B | S.RNLTKDR.C | 1 | 1 |
| Ovalbumin | K.YNLTSVLMAMGITDVFSSSANLSGISSAESLK.I | 2 | 2 |

*The consensus sequence of N-glycosylation sites is highlighted, and the period indicates the peptide cleavage site.

The remaining five N-glycosylation sites reside in large tryptic peptides with molecular weights above 3000 Da which were absent from the LTQ results. The absence of some of the large tryptic peptides with N-linked glycosylation sites is likely to be caused by insufficient ionization of these peptides.

To evaluate the capture efficiency, the total non-captured peptides from the five glycoproteins were analyzed by LTQ after applying PNGase F deglycosylation. Only one N-linked consensus sequence NLS from ovalbumin, was identified, and all the other N-linked sequences were absent. This sequence from ovalbumin has been reported to be a transient glycosylation site that only occurs in hen oviduct (Suzuki et al., Proc. Natl. Acad. Sci. USA 94:6244-6249(1997)). Because of the absence of all other glycosylation sequences from the five glycoproteins in the non-captured peptide mixture, the presence of this N-linked sequence in both the captured and non-captured fraction was likely due to the fact that it is partially glycosylated rather than a result of incomplete capture. The above results suggest that the glyco-peptide-capture approach can be used to comprehensively capture and identify most, and perhaps all, glycopeptides in mixtures.

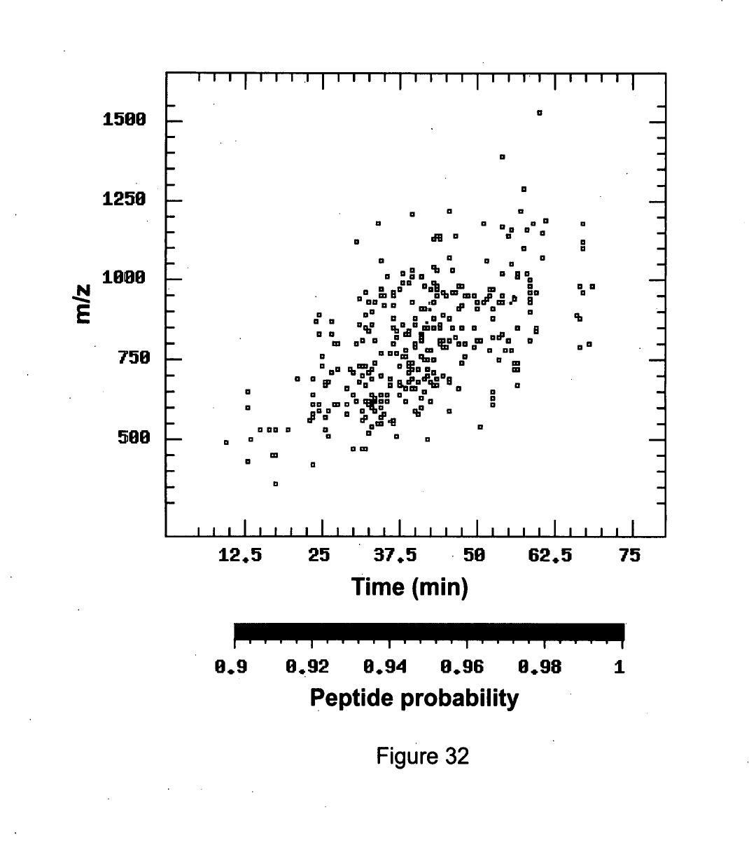

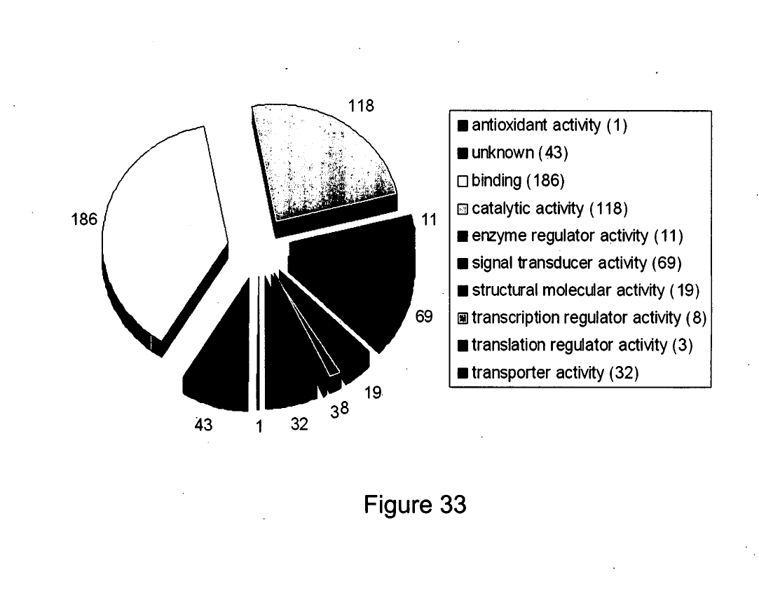

Application of glycopeptide-capture approach to ovarian cell microsomal fractions. Analysis of membrane proteins by MS is challenging because the proteins easily aggregate and are difficult to dissolve in aqueous solutions (Han et al., Nat. Biotechnol. 19:946-951 (2001); Wu et al., Nat. Biotechnol. 21:532-538 (2003)). To assess the applicability of the glycopeptide-capture approach to membrane proteins, the microsomal fraction from a cisplatin-resistant ovarian-cancer cell line (IGROV-1/CP) that is rich in membrane proteins was analzyed. The capture strategy was carried out on two microsomal fractions with 500 μg and 800 μg crude protein, respectively, and one fifth of the final captured peptides were analyzed by a single nanoLC-ESI-MS/MS analysis. Two MS analyses for each of the two capture procedures were performed. In a single MS analysis, 311 unique peptides were unambiguously identified that mapped to 156 unique proteins. FIG. 32 shows the pep3D result (Li et al., Anal. Chem. 76:3856-3860 (2004); Li et al., Mol. Cell. Proteomics 4:1328-40(2005)) of the identified peptides with peptide probability value greater than 0.9. Among the 156 identified proteins, 68 proteins were identified with more than one peptide; and among the 311 identified peptides, 286 peptides have the N-X-T/S consensus sequence. The glycopeptide selectivity of the approach is as high as 91% based on the number of peptides with the N-linked consensus sequence compared to the total number of identified peptides. Combining the results of all four MS runs (two biological replicates), a total of 302 proteins were identified from the microsomal fractions of IGROV-1/CP with an average identification rate of 136±19 (n=4) proteins and glycopeptide selectivity (the probability of the peptide identified have the N-glyco consensus sequence over the total identified peptides) of 91.0±1.6% (n=4) per MS analysis.

Based on the results of previous studies, glycosylated tryptic peptides constitute 2-5% of the glycoproteins (Alvarez-Manilla et al., J. Proteome Res. 5:701-708 (2006); Kaji et al., Nat. Biotechnol. 21:667-672 (2003)). If the enrichment is characterized by the ratio between the percentage of glycopeptides in the sample after (91%) and before (2-5%), assuming all the microsomal proteins are glycoproteins) the capture, then the enrichment factor of the glyco-peptide-capture approach is 19 to 45 fold. As cell microsomal fractions also include organelle and plasma proteins that are not glycosylated (Han et al., Nat. Biotechnol. 19:946-951 (2001)), the enrichment factor estimated here is conservative and provides a good demonstration of the effectiveness of the capture approach in enrichment of glycopeptides from a complex biological sample.

To classify the identified proteins by cellular function and to explore the biological significance of the glycoproteins identified, the data was annotated using GoMiner (discover.nci.nih.gov/gominer) (Zeeberg et al., Genome Biol. 4:R28 (2003)). For the analysis, Entrez Gene names were retrieved from the EMBL International Protein Index (IPI) number, and redundant IDs were removed before GoMiner analysis. Among 302 proteins with Entrez Gene names, 251 proteins have been annotated as cellular components, and 244 proteins have been annotated for molecular ftmction. As expected, the majority of identified proteins are membrane proteins (170 out of251). The major molecular functions among the identified proteins include ligand binding, catalytic activity, signal transduction activity, transporter activity, etc. (shown in FIG. 44). The results of this analysis are concordant with knowledge of the main cellular location and functions of glycoproteins in the microsomal fractions.

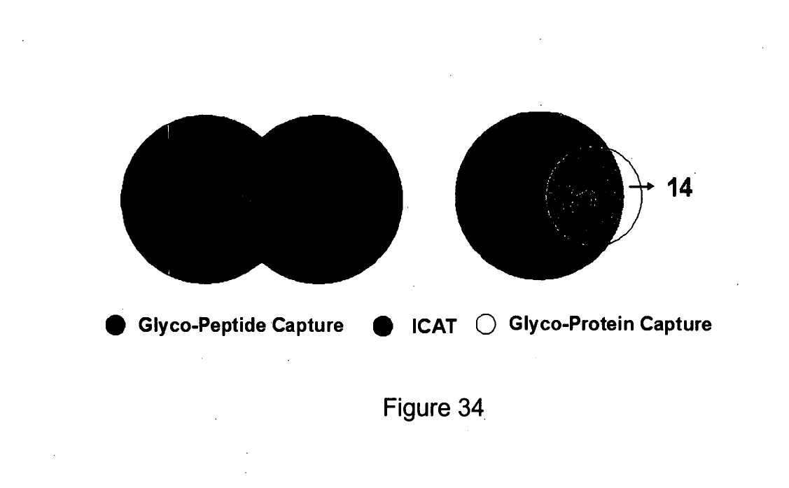

The proteins identified were compared to the proteins identified previously by the ICAT/MS/MS approach on a similar microsomal fraction of IGROV-1/CP cells (Stewart et al., Mol. Cell. Proteomics 5:433-443 (2006)). Interestingly, only 46 proteins overlapped in the two datasets (302 proteins identified in glycopeptide-capture dataset and 307 in ICAT dataset), suggesting that these two approaches allow detection of different subsets of the microsomal proteome (shown in FIG. 34) and complement each other for global proteomics. The identified proteins were also compared with Zhang's glycoprotein list obtained from microsomal fractions of the prostate-cancer epithelial cell line, LNCaP (described in Zhang et al., Nat. Biotechnol. 21:660-606 (2003)). Notwithstanding the difference in cell lines, the two microsomal glycoprotein datasets share 50 proteins in common (302 proteins in peptide-capture dataset whereas only 64 proteins in protein-capture dataset). That is 77% of the total identified proteins Zhang et al., supra, 2003, but is only 31% of the total identified proteins here (FIG. 34). Most shared proteins are common and abundant glycoproteins in cell microsomal fractions, such as integrin, sodium/potassium-transporting ATPase, cation-independent mannose-6-phosphate receptor, lysosome-associated membrane glycoprotein, glucuronidase, glycosidase, mannosidase, hexosaminidase, glucosyeramidase, and the like. The much larger protein dataset including more biologically and clinically interesting glycoproteins obtained from the capture approach indicates that a more comprehensive identification of glycoproteome was achieved. At the same time, more glycosylation sites from the same protein were identified by our glycopeptide-capture approach than from the protein-capture approach. For example, using glycoprotein-capture, only one glycosylation site has been identified from gamma-1 chain of laminin protein (Zhang et al., supra, 2003), while using the glycopeptide-capture approach, another 5 additional glycosylation sites were been identified.

The biological and clinical importance of glycoproteins raises the need for a comprehensive and robust molecular profiling of glycoproteins, and accordingly, ways to enrich glycoproteins. Moreover, system approaches to biology and medicine benefit enormously from global (comprehensive) approaches to the analysis of genes and proteins. An approach has been developed that digests proteins into peptides and enriches glycopeptides via hydrazide chemistry. Adapting the pros and avoiding the cons raised from both the “top down” and “bottom up” strategies, the enrichment approach is designed to have improved robustness and completeness for glycoproteomics. This approach appears to move us toward more global analyses of glycoproteins. The advantages of the approach over the existing methods are elaborated below:

First, digestion of proteins into peptides improves solubility of large membrane proteins and exposes all of the glycosylation sites to ensure equal accessibility to external capture reagents. Analyses of known structures of glycoproteins indicate that about one third of glycosylation sites are buried inside of proteins (Petrescu et al., Glycobiology 14:103-114 (2004)). Therefore, the steric hindrance raised from protein topology can diminish the capture efficiency of many sites of glycosylation. Cleaving globular proteins to smaller peptides circumvents this shortcoming.

Second, capturing glycosylated peptides can effectively reduce the complexity of the sample and increase the confidence of using MS-based protein identifications. Even though the protein-capture strategy can effectively enrich glycoproteins from complicated samples, the peptides (20 or more tryptic peptides per protein) generated from proteolysis prior to MS analysis increase the sample complexity again and counteract the enrichment effect at the protein level. Given the fact that proteins can be identified by individual signature proteolytic peptides with MS, and that identification from multiple peptides improves the confidence of protein assignment (Nesvizhskii et al., Anal. Chem. 75:4646-4658 (2003)), it is ideal to use multiple peptides to identify a protein. Because glycoproteins are glycosylated at multiple sites in general (Kaji et al., Nat. Biotechnol. 21:667-672 (2003); Zhang et al., supra, 2003) and because the glycopeptides constitute only 2-5% of the full glycoprotein, enriching glycopeptides not only decreases sample complexity effectively, but also provides multiple peptides for unambiguous protein identification. Using 0.9 as the protein probability cutoff score, on average the error rate was as small as 0.006 in all four MS runs, and the number of incorrectly identified peptides was 1 out of 136 by statistical analysis (Nesvizhskii et al., Anal. Chem. 75:4646-4658 (2003)).

Third, the capture approach using hydrazide chemistry provides good selectivity of glycopeptides over the non-glycosylated peptides. To date, different chromatographic separation techniques have been reported to enrich glycopeptides by the diverse physical and chemical properties of the glycopeptides (Alvarez-Manilla et al., J. Proteome Res. 5:701-708 (2006); An et al., Anal. Chem. 75:5628-5637 (2003); Hagglund et al., J. Proteome Res. 3:556-566 (2004); Larsen et al., Mol. Cell. Proteomics 4:107-119 (2005); Wada et al., Anal. Chem. 76:6560-6565 (2004)). The selectivity, however, is very limited (Alvarez-Manilla et al.,supra, 2006; Hagglund et al., supra, 2004). To overcome this problem, hydrazide chemistry was used, which allows selective capturing of glycopeptides via a covalent bond formed between hydrazide and the aldehyde groups from oligosaccharides. Such chemistry is ubiquitous to all glycan structures, and the covalent bond formed tolerates extreme wash conditions. Virtually all non-covalently attached peptides can be removed from the solid support. Using the chemical capture approach, a glycopeptide selectivity of 91% has been achieved on the microsomal fraction of the ovarian-cancer cells.

Fourth, the utility of sodium-sulphite as a quencher in the capture approach in place of the SPE (solid phase extraction) step in the glycoprotein chemical-capture approach, which removes the excess sodium periodate, allows the overall capture procedure to be completed in a single vessel. This modification prevents sample loss as well as saving labor and time. Sample loss is a nontrivial problem when the proteomics research is focused on low-abundant proteins such as biomarkers. The more than 3 fold increase in the number of identified proteins by the approach compared to the glycoprotein capture approach described in Zhang et al., supra, 2003, may in part due to the avoidance of sample losses. Another reason for the limited yield in the glycoprotein capture approach described in Zhang et al., supra 2003, is due to the incomplete capture of glycopeptides inherent in the protein-capture approach itself as illustrated in FIG. 30A and 30B. For a multi-glycosylated protein, it is highly unlikely that all of the glycosylated sites can form chemical bonds with the solid support due to the globular structure of proteins (FIG. 30A). After on-support proteolysis and a series of washes to remove non-bonded peptides, only a fraction of the glycopeptides remains on the support (FIG. 30B). For example, using glycoprotein captur,e only one glycosylation site was identified from gamma-1 chain of laminin protein (Zhang et al., supra, 2003); whereas using the glycopeptide-capture approach, 5 additional glycosylation sites were identified. As the peptide-prophet and protein-prophet analyses penalize single-hit identifications and reward multi-hit identifications (Nesvizhskii et al., Anal. Chem. 75:4646-4658 (2001)), the glycoprotein capture approach is likely to result in a lower protein identification rate (64 proteins in total) compared with the glycopeptide capture approach described in this example (302 proteins in total).

The glycopeptide capture approach is adaptable to high throughput and automation because of the completion of capture in a single vessel. The first-step proteolysis in the peptide chemical capture approach is compatible with quantitative proteomic analyses. Moreover, the glycopeptide capture approach is complementary to the widely used ICAT approach that labels and enriches cysteine-containing peptides. With only a small fraction of the peptides overlapping, the number of proteins identified by the glycopeptide-capture approach is similar to that of the ICAT approach. A total of 569 proteins were identified from the microsomal fractions of IGROV-1/CP by combining the ICAT and glycopeptide capture results, which indicates that the use of both strategies in concert provides a powerful approach to global proteomic profiling of complex biological medium.

Although the biological significance of the proteins identified in this study is not the focus of this study, many of the glycoproteins identified have been implicated in ovarian carcinoma and cisplatin resistance. For instance, the folate receptor (Hilgenbrink et al., J. Pharm. Sci. 94:2135-2146 (2005); Holm et al., Biosci. Rep. 18:49-57 (1998)), the insulin like growth factor receptor (Dal Maso et al., Oncology 67:225-230 (2004)), and the epidermal growth factor receptor (Oliveira et al., Expert Opin. Biol. Ther. 6:605-617 (2006)) are over expressed in cancer cells and are used as drug delivery targets. The tumor-associated calcium signal transducer 1 (Gagne et al., Mol. Cell. Biochem. 275: 25-55 (2005)), tumor necrosis factor receptor (Gagne et al., Mol. Cell. Biochem. 275:25-55(2005); Debemardis et al., J. Pharmacol. Exp. Ther. 279:84-90 (1996); Kulbe et al., Cancer Res. 65:10355-10362 (2005)), metastasis suppressor protein 1 (Gagne et al., supra, 2005), heat shock protein HSP 90 (48), laminin (Gagne et al., supra, 2005; Shin et al., J. Biol. Chem. 278:7607-7616 (2003); Yow et al., Proc. Natl. Acad. Sci. USA 85:6394-6398(1988)), and reticulocalbin-1 (Hirano et al., Int. J. Cancer 117:460-468 (2005)) have been reported to be associated with ovarian carcinogenesis. Increased expression of disulfide-isomerase (Gagne et al., supra, 2005; Stierum et al., Biochim. Biophys. Acta 1650:73-91 (2003)), and ADAM 10 (Gough et al., J. Immunol. 172:3678-3685 (2004)) is strongly correlated with cisplatin resistance. These observations validate the contention that the peptide glycocapure method d is a powerful approach to the discovery of potential biomarkers. Meanwhile, CD proteins that play important immune functions in cells are a class of membrane proteins which are often glycosylated and also make good drug targets and biomarkers. The protein dataset was compared with the PROW database for CD proteins (mpr.nci.nih.gov/prow/) (361 CD proteins in total) and identified 74 CD proteins.

Even though this approach can also capture O-linked glycosylated peptides, for ease of analysis the N-linked glycopeptides only were detected. With a proper combination of O-glycosidase or chemical cleavage such as β-elimination, the O-glycopeptides can also be released from the solid support and analyzed by MS. Due to technical limitations of MS analysis, such as ionization efficiency of peptides; sample complexity and dynamic range; and mass accuracy and resolution of mass spectrometry itself (Aebersold and Cravatt, Trends Biotechnol. 20:S1-2 (2002); Anderson et al., Mol. Cell. Proteomics 1:845-867 (2002); Diamandis, Mol. Cell. Proteomics 3:367-378 (2004)), not all the tryptic glycopeptides can be detected. To use this approach to study the glycosylation site(s) of individual proteins for the purpose of investigating post translational modifications, detection approaches other than MS would be necessary. Nonetheless, to serve the purpose of global glycoproteomics, this strategy provides a comprehensive and robust methodology with improved accuracy and sensitivity.

### EXAMPLE XVIII

### Glycopeptide Capture

Additional experiments were performed on human sera and a cell line extract each containing an internal glycosylated standard (bovine fetuin) to assess the effective yield of each procedure.

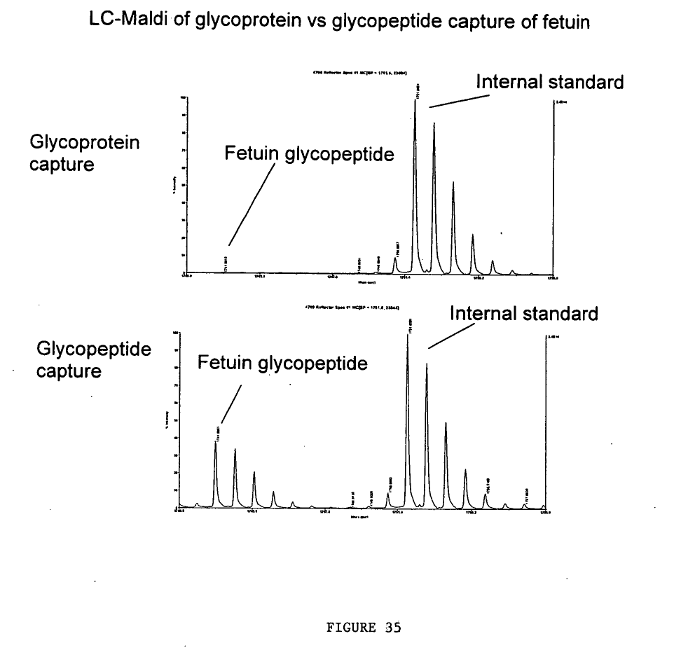

Briefly, fetuin was spiked into two background protein mixtures (CL1 cell lysate and serum) such that fetuin was 5% by weight. Each sample (CL1 and serum) was split into two fractions, where one was subjected to the glycoprotein capture as described in Example I, and the other was subjected to the glycopeptide capture method as described in Example XVII except that it was performed in the absence of RapiGest™ and sodium sulfite. 96 pmol of a stable isotope labelled fetuin peptide (LCPDCPLLAPLDDSR, with carbamidomethylated cysteine and ¹³C and ¹⁵N labelling of the C-terminal R) containing the N-linked site (but with the N converted to D) was spiked into the samples that contained 1092 pmol of fetuin. The samples containing the internal standard were subjected to solid phase extraction prior to MALDI-TOF analysis. Comparing the ratios of ion abundances of the internal standard versus fetuin peptide for glycopeptide and glycoprotein capture showed that the glycopeptide capture had a 20-30 fold higher yield (same results for serum or CL1 background)(see FIG. 35). Similar results were obtained when analyzed by LC-MALDI

In more detail, the glycoprotein capture method was performed as follows. An aliquot of 60 μL MARS depleted serum (600 μgs) or 94 μL CL-1 extract (600 μgs) were diluted with 16 μL 10×coupling buffer (50 mM EDTA, 400 mM Tris, pH 8.0), 6 μL fetuin and water to 166 μL. Samples were oxidized using 4 μL 10 mg/mL sodium periodate to convert vicinal diols to aldehydes fro 30 min at RT. The oxidized sample was applied to a 500 μL preequilibrated hydrazine beads (50% slurry) and incubated overnight at RT with end over end mixing. Beads were collected by centrifugation and washed thoroughly in denaturing buffer (8 M urea and 400 mM ammonium bicarbonate) to remove unbound proteins. Bound proteins were reduced with a final concentration of 8 mM TCEP for 30 minutes at room temperature. Reduced cysteines were alkylated with 10.6 mM iodoacetamide for 30 min at room temperature. Beads were thoroughly washed three times in 1 mL denaturing buffer and proteins trypsinized overnight at 37° C. Supernatant fractions were collected and beads washed extensively with 1 mL each of 1.5M NaCl, 80% acetonitrile (ACN), 100% methanol (MeOH), water and 100 mM ammonium bicarbonate. Bound glycopeptides were then enzymatically cleaved from the hydrazide resin with PNGaseF overnight at 37° C. Supernatant fractions were collected. Beads were washed with 80% ACN and combined with initial elution. Samples were dried to completion in a Speedvac. Samples were resuspended in buffer A and processed for mass spectrometric analyses.

For glycopeptide capture, 60 μL MARS depleted serum (600 μgs) or 94 μL CL-1 extract (600 μgs) were diluted with 16 μL 10×coupling buffer (50 mM EDTA, 400 mM Tris pH 8.0), 6 μL fetuin and water to 166 μL. To this was added 4 μL 500 mM TCEP (10 mM final concentration) and incubate at RT 30 min. To this was added 96 mg urea for 30 min at RT. To this was added 4 μL of 250 mM iodoacetamide for 30 min at RT. To this was added 0.5 μL IM DTT for 20 min at RT. The urea in the sample was diluted by adding 1 mL 40 mM Tris pH 8.0. To this was added 10 μg sequencing grade trypsin and incubated with constant mixing overnight at 37° C. The sample was acidified by adding 25 μL 10% TFA, and the pH checked using paper strips.

For reverse phase cleanup of the sample, a Hydrate C-18 spin column (Harvard Apparatus Macrospin) with 500 μL 60% ACN, 0.1% TFA was used. The column was washed three times with 500 μL 2% ACN, 0.1% TFA. The sample was loaded and spun and the sample was passaged twice to collect all protein. The column was washed three times with 200 μL 0.1% TFA. Proteins were eluted with 3×75 μL of 60% ACN, 0.1% TFA. The eluate was collected and spun dry in a Speedvac. Dried peptides were resuspended in 160 μL 1×coupling buffer. To this was added 40 μL 10 mg/mL sodium periodate for 30 min at RT. Oxidized sample was added to 500 μL of pre-equilibrated hydrazide beads (50% slurry in coupling buffer) and incubated at RT overnight with constant mixing. Unbound fractions were collected and stored. Bound proteins (resin) were washed 2×1 mL each of water, 1.5 M NaCl, methanol, 80% ACN, 100 mM AMBIC. After the final wash, the beads were resuspend beads in 300 μL of 100 mM AMBIC containing 1 μL PNGaseF and incubated overnight at 37oC. with constant agitation. The supernatant fraction was collected and transferred to fresh tubes. The resin was washed 2×100 μL 80% ACN, collecting the washes each time and transferring to the eluted fraction. The sample was dried down in a Speedvac. The samples were resuspended in water and desalted using reverse phase column prior to SCX and MS analyses.

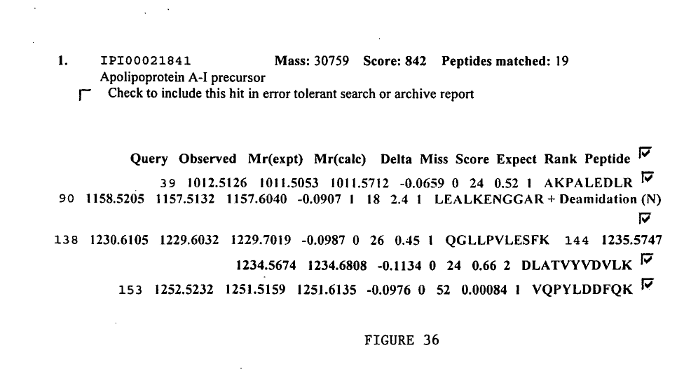

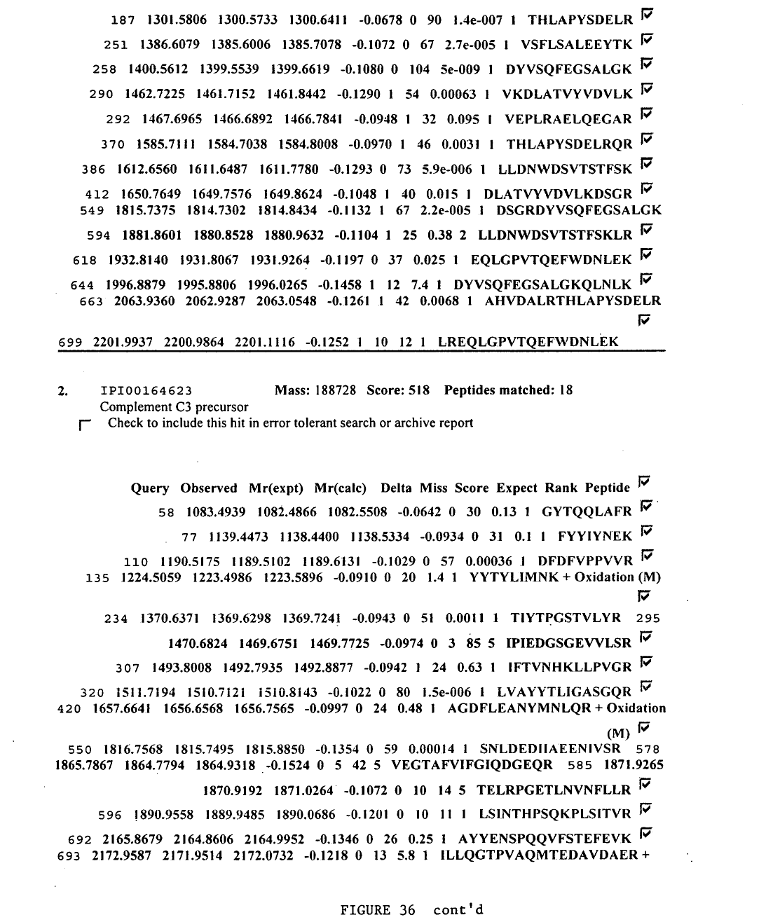

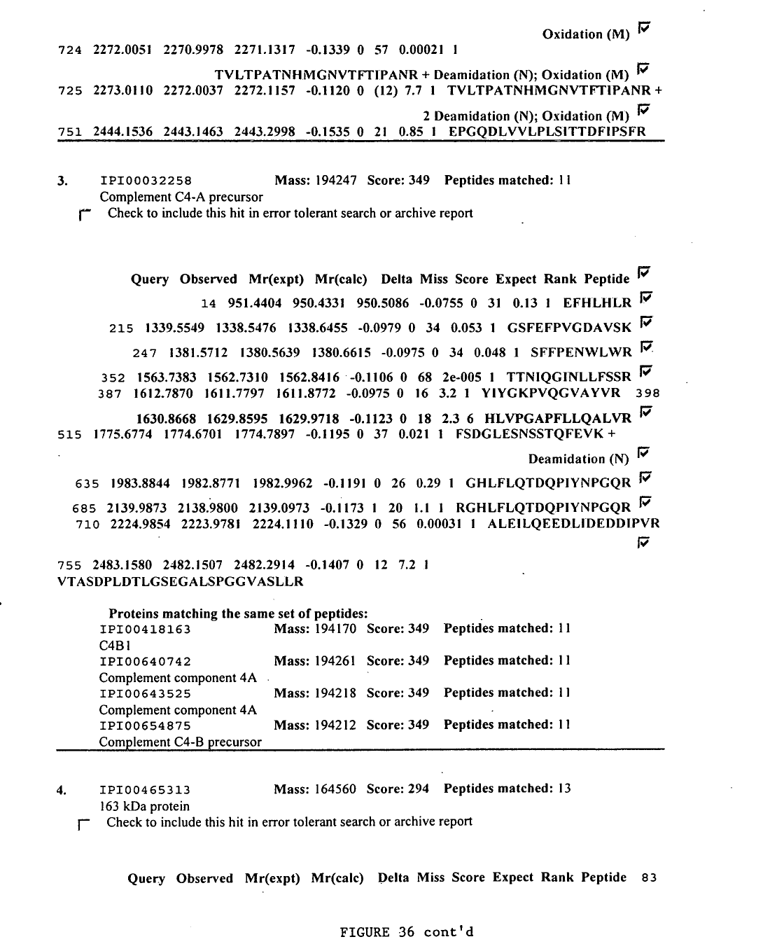

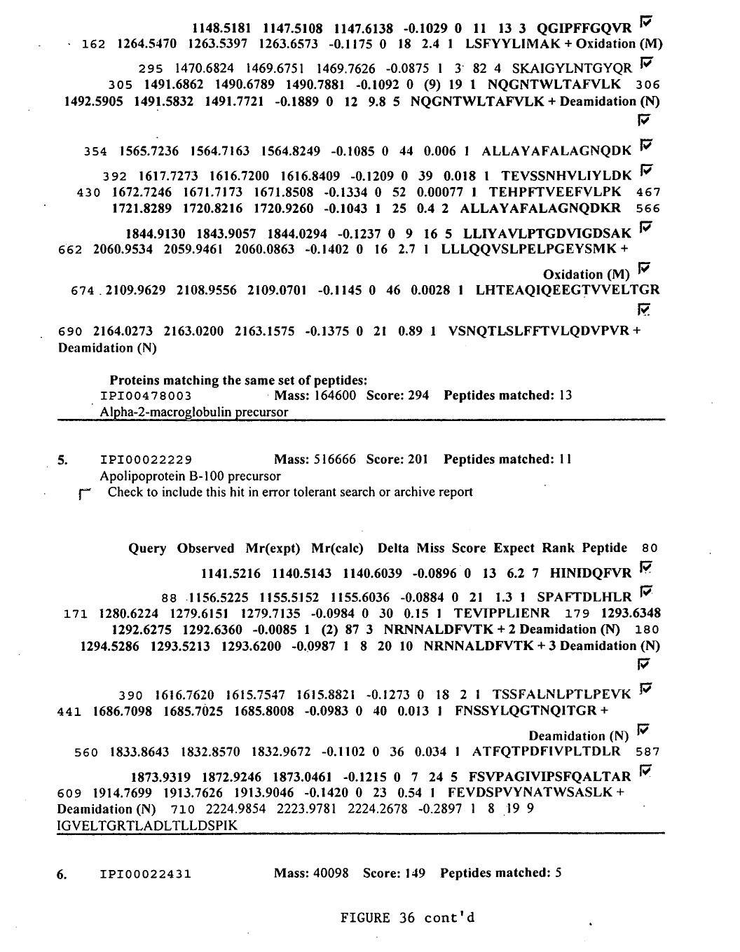

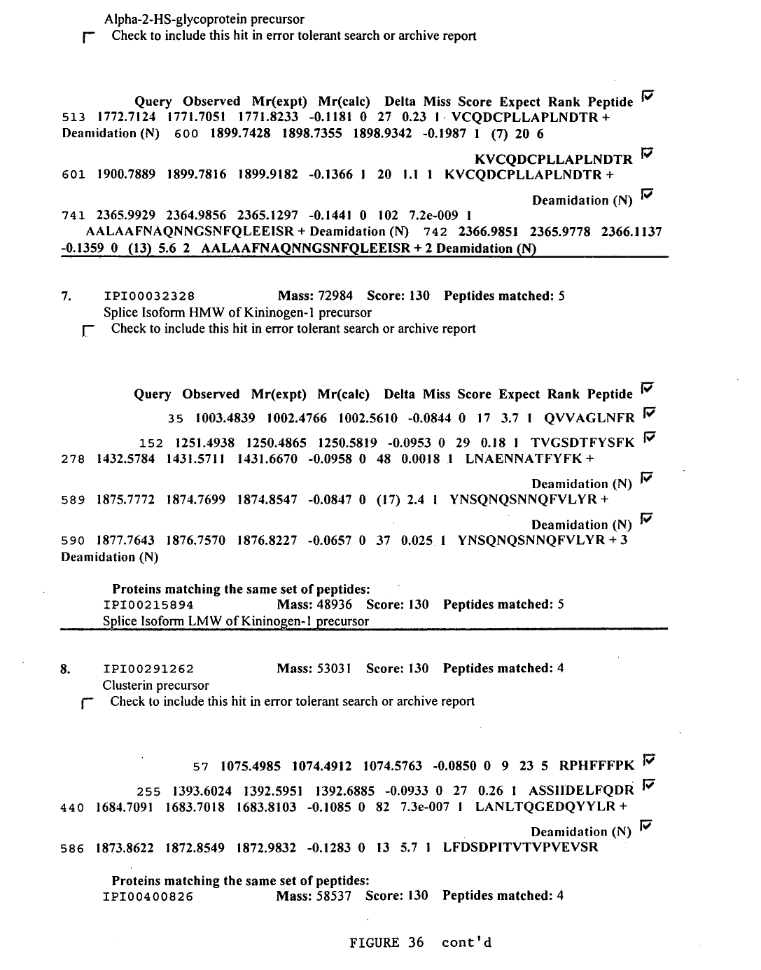

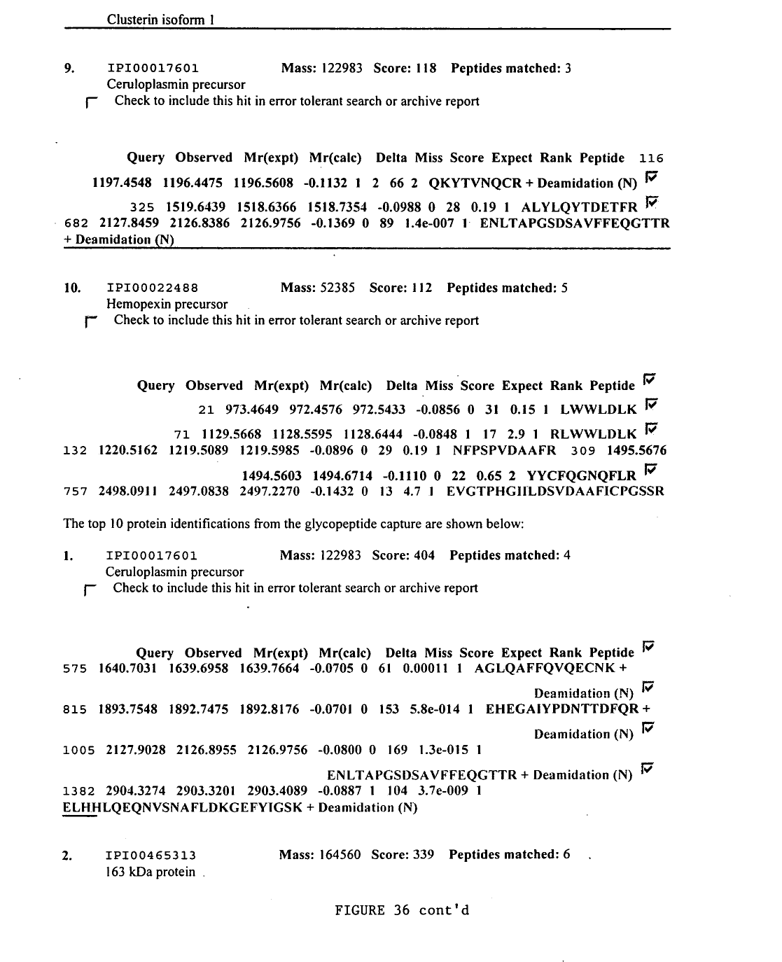

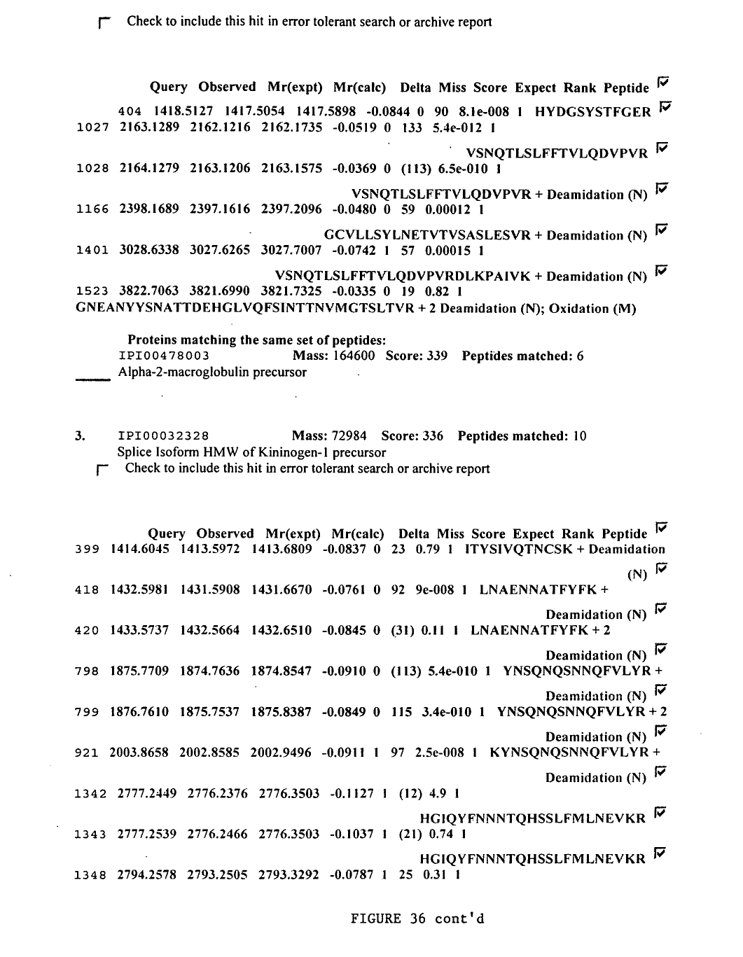

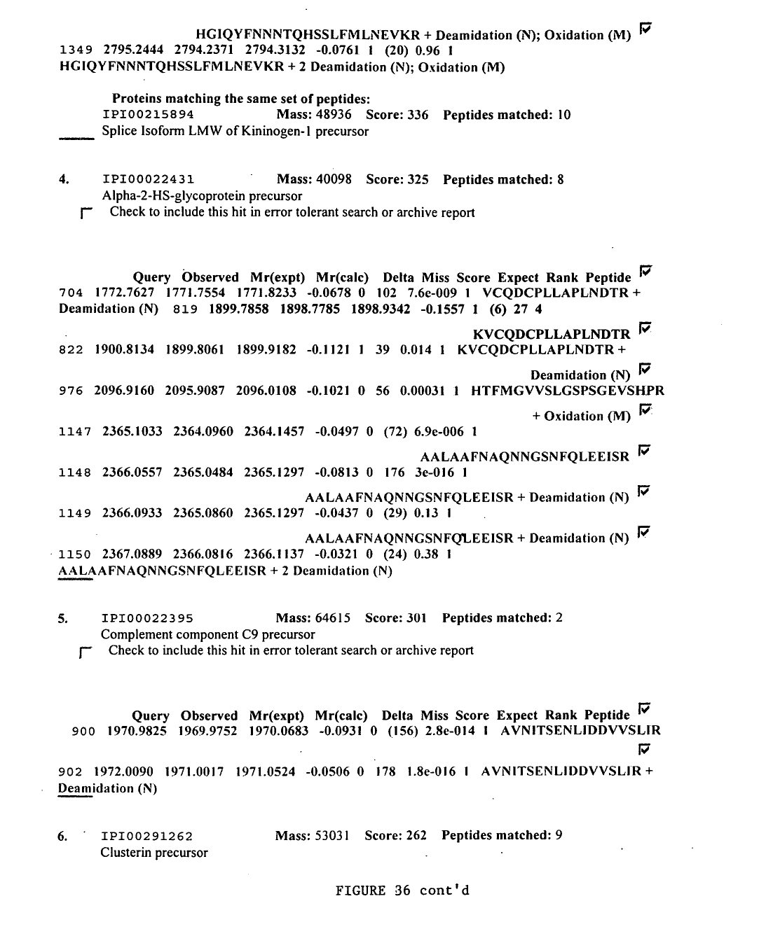

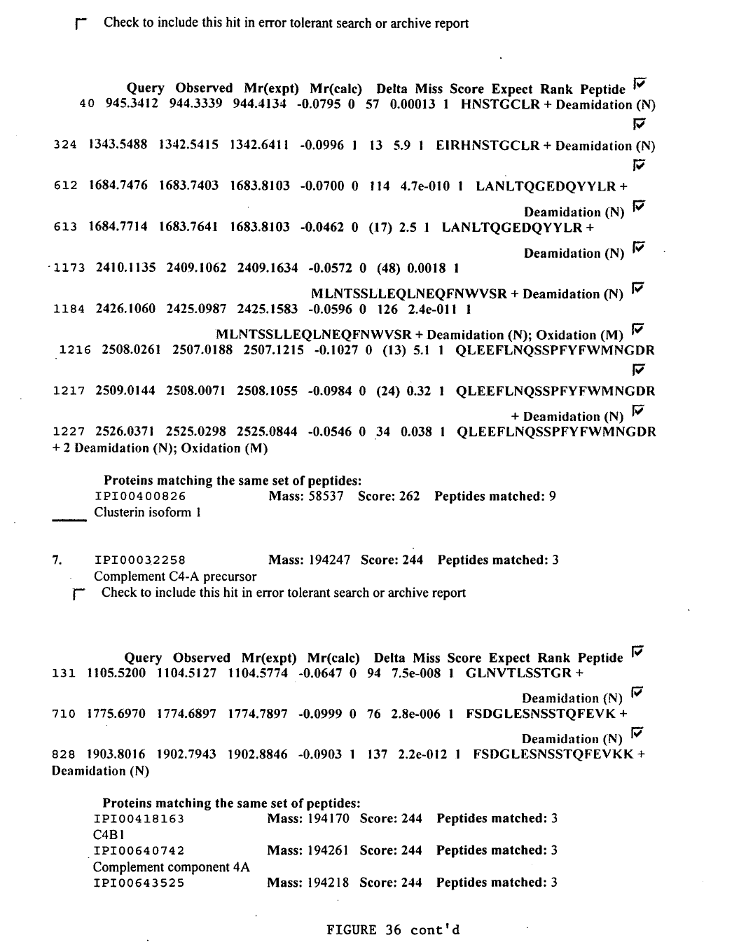

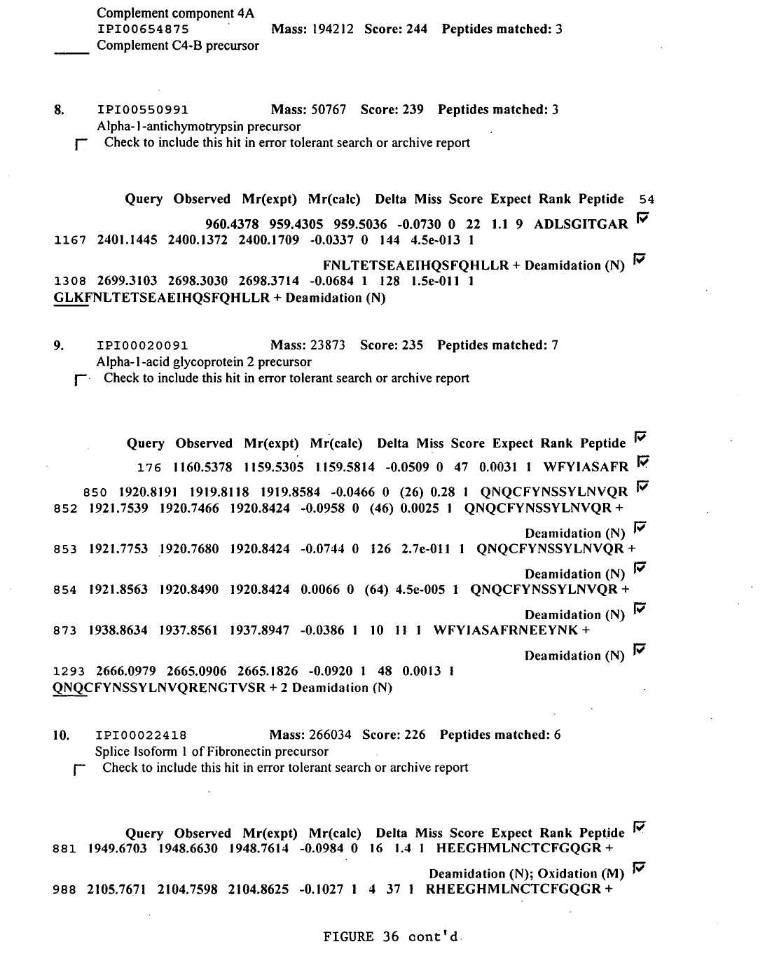

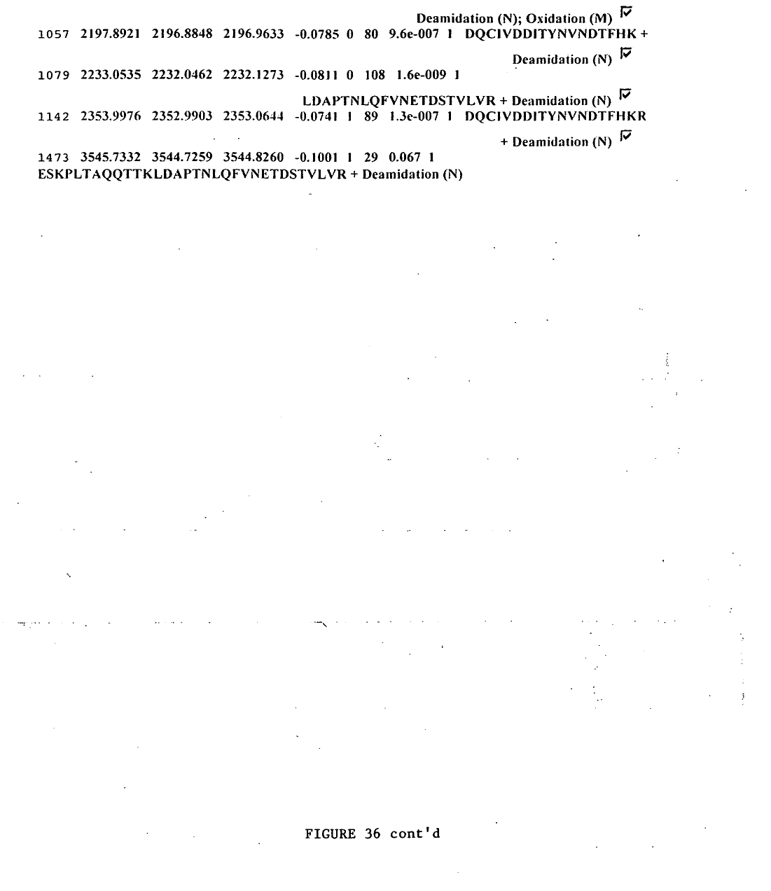

The serum glycoprotein and glycopeptide captures were also analyzed by LC-MS/MS using the 4800 Maldi TOF-TOF, and the resulting MSMS spectra obtained by data dependent analysis. The MS/MS spectra were identified using Mascot. The top 10 entries for the glycoprotein capture are shown in FIG. 36.

The results show that there are a large number of non-glycosylated peptides in the serum glycoprotein capture, but very little in the glycopeptide capture, that is, the selectivity is higher. Also, the scores in the glycopeptide capture are much higher than for the same peptides in the glycoprotein capture, which is most likely due to higher intensity precursor ions resulting from higher capture yields. One final point is that although glycopeptides containing N-terminal Ser or Thr are present in the glycoprotein capture list, they are absent from the glycopeptide list. This is most likely due to oxidation of the vicinal amino and hydroxyl groups. It should be noted that this reaction could be eliminated by first derivatizing amino groups; a procedure that is amenable to isotope coded labelling procedures like ITRAQ.

These experiments indicate that glycopeptide capture is has advantages over glycoprotein capture with respect to yield and specificity of capture. Indeed, a direct comparison of the two procedures indicates a 20-30 fold higher yield than the glycoprotein method. The absolute yield for each of the procedures remains to be determined. With respect to specificity of glycopeptide identification, the peptides derived from the top twenty identified proteins were examined from each procedure from a serum sample. Glycoprotein capture resulted in the identification of 40 peptides with high confidence, of these 13 contained the N-X-S glycosylation motif, a specificity of 33%. Glycopeptide capture identified 50 peptides containing a consensus glycosylation site from 45 identified peptides (90% specificity). A more pronounced difference was observed for CLI whole cell lysates, where none of the peptides from a glycoprotein capture experiment contained N-linked consensus sites, whereas the opposite was nearly true for glycopeptide capture (2 out of 27 were not glycopeptides). Both of these findings, higher yield and specificity, are a significant advancement to the technology of glycocapture. It should be noted that glycopeptides containing N-terminal Ser or Thr cannot be identified by the glycopeptide capture approach, since periodate converts the Ser or Thr to an aldehyde that either is dispersed via reactions with side chains from other peptides, or is permanently attached to the hydrazide bead. As such, no N-terminal Ser nor Thr containing peptides were identified by this method. Furthermore, data exists showing the presence of the oxidized Ser on specific peptides (both MS and MSMS). As discussed in Example XVII, the use of an oxidant quencher like sodium sulphite, and the detergent Rapigest, may lead to an increase in both yield and efficacy of the glycopeptide capture and possibly the glycoprotein capture procedure.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method for identifying and quantifying glycopolypeptides in a sample, comprising:
(a) derivatizing glycopolypeptides in a polypeptide sample;
(b) immobilizing said derivatized glycopolypeptides to a solid support;
(c) cleaving said immobilized glycopolypeptides, thereby releasing non-glycosylated peptide fragments and retaining immobilized glycopeptide fragments;
(d) labeling said immobilized glycopeptide fragments with an isotope tag;
(e) releasing said glycopeptide fragments from said solid support, thereby generating released glycopeptide fragments;
(f) analyzing said released glycopeptide fragments using mass spectrometry;
(g) identifying a released glycopeptide fragment; and
(h) quantifying the amount of said glycopeptide fragment identified in step (g).

2. The method of claim 1, wherein said solid support comprises a hydrazide moiety.

3. The method of claim 1, wherein said glycopeptides are released from said solid support using a glycosidase.

4. The method of claim 3, wherein said glycosidase is an N-glycosidase or an O-glycosidase.

5. The method of claim 4, wherein said glycopeptides are released from said solid using sequential addition of N-glycosidase and O-glycosidase.

6. The method of claim 1, wherein said glycopeptides are released from said solid support using chemical cleavage.

7. The method of claim 1, wherein said glycopolypeptides are oxidized with periodate.

8. The method of claim 1, wherein said glycopolypeptides are cleaved with trypsin.

9. The method of claim 1, wherein said released non-glycosylated peptides are isotopically labeled and analyzed by mass spectrometry.

10. The method of claim 1, wherein said sample is selected from a body fluid, secreted proteins, and cell surface proteins.

11. A method for identifying and quantifying glycopeptides in a sample, comprising:
(a) immobilizing glycopolypeptides to a solid support;
(b) cleaving said immobilized glycopolypeptides, thereby releasing non-glycosylated peptides and retaining immobilized glycopeptides;
(c) labeling said immobilized glycopeptides with an isotope tag;
(d) releasing said glycopeptides from said solid support; and
(e) analyzing said released glycopeptides.

12. The method of claim 11, wherein said glycopolypeptides are oxidized.

13. The method of claim 12, wherein said solid support comprises a hydrazide moiety.

14. The method of claim 12, wherein said glycopolypeptides are oxidized with periodate.

15. The method of claim 11, wherein said glycopeptides are released from said solid support using a glycosidase.

16. The method of claim 15, wherein said glycosidase is an N-glycosidase or an O-glycosidase.

17. The method of claim 16, wherein said glycopeptides are released from said solid using sequential addition of N-glycosidase and O-glycosidase.

18. The method of claim 11, wherein said glycopeptides are released from said solid support using chemical cleavage.

19. The method of claim 11, wherein said glycopolypeptides are cleaved with trypsin.

20. The method of claim 11, wherein said released non-glycosylated peptides are analyzed by mass spectrometry.

21. A method for identifying and quantifying glycopeptides in a sample, comprising:
(a) of immobilizing glycopolypeptides to a solid support;
(b) cleaving said immobilized glycopolypeptides, thereby releasing non-glycosylated peptides and retaining immobilized glycopeptides;
(c) releasing said glycopeptides from the solid support; and
(d) analyzing said released glycopeptides.

22. The method of claim 21, wherein said glycopolypeptides are oxidized.

23. The method of claim 22, wherein said solid support comprises a hydrazide moiety.

24. The method of claim 22, wherein said glycopolypeptides are oxidized with periodate.

25. The method of claim 21, wherein said glycopeptides are released from said solid support using a glycosidase.

26. The method of claim 25, wherein said glycosidase is an N-glycosidase or an O-glycosidase.

27. The method of claim 26, wherein said glycopeptides are released from said solid using sequential addition of N-glycosidase and O-glycosidase.

28. The method of claim 21, wherein said glycopeptides are released from said solid support using chemical cleavage.

29. The method of claim 21, wherein said glycopolypeptides are cleaved with trypsin.

30. The method of claim 21, wherein said released non-glycosylated peptides are analyzed by mass spectrometry.

31. A method of identifying a diagnostic marker for a disease, comprising:
(a) immobilizing glycopolypeptides from a test sample to a first solid support;
(b) immobilizing glycopolypeptides from a control sample to a second solid support;
(c) cleaving said immobilized glycopolypeptides, thereby releasing non-glycosylated peptides and retaining immobilized glycopeptides;
(d) labeling said immobilized glycopeptides on said first and second supports with differential isotope tags on the respective supports;
(e) releasing the glycopeptides from said solid supports;
(f) analyzing the released glycopeptides; and
(g) identifying one or more glycosylated polypeptides having differential glycosylation between the test sample and the control sample.

32. The method of claim 31, wherein said glycopolypeptides are oxidized.

33. The method of claim 32, wherein said solid support comprises a hydrazide moiety.

34. The method of claim 32, wherein said glycopolypeptides are oxidized with periodate.

35. The method of claim 31, wherein said glycopeptides are released from said solid support using a glycosidase.

36. The method of claim 35, wherein said glycosidase is an N-glycosidase or an O-glycosidase.

37. The method of claim 36, wherein said glycopeptides are released from said solid using sequential addition of N-glycosidase and O-glycosidase.

38. The method of claim 31, wherein said glycopeptides are released from said solid support using chemical cleavage.

39. The method of claim 31, wherein said glycopolypeptides are cleaved with trypsin.

40. The method of claim 31, wherein said released non-glycosylated peptides are analyzed by mass spectrometry.

41. The method of claim 31, wherein the disease is cancer.

42. A kit comprising a hydrazide resin, periodate, and a pair of differentially labeled isotope tags.

43. A method for identifying glycopolypeptides in a sample, comprising:
(a) cleaving glycopolypeptides to generate glycopeptide fragments;
(b) derivatizing said glycopeptide fragments in a polypeptide sample;
(c) immobilizing said derivatized glycopeptide fragments to a solid support;
(d) releasing said glycopeptide fragments from said solid support, thereby generating released glycopeptide fragments;
(e) analyzing said released glycopeptide fragments using mass spectrometry; and
(f) identifying a released glycopeptide fragment.

44. The method of claim 43, further comprising labeling said immobilized glycopeptide fragments with an isotope tag.

45. The method of claim 44, further comprising quantifying the amount of said identified glycopeptide fragment.

46. The method of claim 43, wherein said solid support comprises a hydrazide moiety.

47. The method of claim 43, wherein said glycopeptide fragments are released from said solid support using a glycosidase.

48. The method of claim 47, wherein said glycosidase is an N-glycosidase or an O-glycosidase.

49. The method of claim 48, wherein said glycopeptide fragments are released from said solid support using sequential addition of N-glycosidase and O-glycosidase.

50. The method of claim 43, wherein said glycopeptide fragments are released from said solid support using chemical cleavage.

51. The method of claim 43, wherein said glycopeptide fragments are oxidized with periodate.

52. The method of claim 43, wherein said glycopolypeptides are cleaved with trypsin.

53. The method of claim 43, wherein said sample is selected from a body fluid, secreted proteins, and cell surface proteins.

54. A method for identifying glycopeptides in a sample, comprising:
(a) cleaving glycopolypeptides to generate glycopeptide fragments;
(b) immobilizing said glycopeptide fragments to a solid support;
(c) releasing said glycopeptide fragments from the solid support; and
(d) analyzing said released glycopeptide fragments.

55. The method of claim 54, further comprising labeling said immobilized glycpeptide fragments with an isotope tag.

56. The method of claim 54, wherein said glycopeptide fragments are oxidized.

57. The method of claim 56, wherein said solid support comprises a hydrazide moiety.

58. The method of claim 56, wherein said glycopeptide fragments are oxidized with periodate.

59. The method of claim 54, wherein said glycopeptide fragments are released from said solid support using a glycosidase.

60. The method of claim 59, wherein said glycosidase is an N-glycosidase or an O-glycosidase.

61. The method of claim 60, wherein said glycopeptide fragments are released from said solid using sequential addition of N-glycosidase and O-glycosidase.

62. The method of claim 54, wherein said glycopeptide fragments are released from said solid support using chemical cleavage.

63. The method of claim 54, wherein said glycopolypeptides are cleaved with trypsin.

64. A method of identifying a diagnostic marker for a disease, comprising:
(a) cleaving glycopolypeptides from a test sample to generate test glycopeptide fragments;
(b) cleaving glycopolypeptides from a control sample to generate control glycopeptide fragments;
(c) immobilizing said test glycopeptide fragments to a first solid support;
(d) immobilizing said control glycopeptide fragments from a control sample to a second solid support;
(e) releasing the test glycopeptide fragments and control glycopeptide fragments from said solid supports;
(f) analyzing the released glycopeptide fragments; and
(g) identifying one or more glycosylated polypeptides having differential glycosylation between the test sample and the control sample.

65. The method of claim 64, further comprising labeling said immobilized glycopeptide fragments on said first and second supports with differential isotope tags on the respective supports.

66. The method of claim 64, wherein said glycopeptide fragments are oxidized.

67. The method of claim 66, wherein said solid support comprises a hydrazide moiety.

68. The method of claim 66, wherein said glycopeptide fragments are oxidized with periodate.

69. The method of claim 64, wherein said glycopeptide fragments are released from said solid support using a glycosidase.

70. The method of claim 69, wherein said glycosidase is an N-glycosidase or an O-glycosidase.

71. The method of claim 70, wherein said glycopeptide fragments are released from said solid support using sequential addition of N-glycosidase and O-glycosidase.

72. The method of claim 64, wherein said glycopeptide fragments are released from said solid support using chemical cleavage.

73. The method of claim 64, wherein said glycopolypeptides are cleaved with trypsin.

74. The method of claim 64, wherein the disease is cancer.

75. A method for identifying glycopolypeptides in a sample, comprising:
(a) adding a detergent to a sample comprising glycopolypeptides;
(b) cleaving glycopolypeptides in said sample to generate glycopeptide fragments;
(c) adding an oxidizing agent to derivatize said glycopeptide fragments;
(d) adding a quencher to quench said oxidizing agent;
(e) immobilizing said derivatized glycopeptide fragments to a solid support;
(f) releasing said glycopeptide fragments from said solid support, thereby generating released glycopeptide fragments;
(g) analyzing said released glycopeptide fragments using mass spectrometry; and
(h) identifying a released glycopeptide fragment.

76. The method of claim 75, further comprising labeling said immobilized glycopeptide fragments with an isotope tag.

77. The method of claim 76, further comprising quantifying the amount of said identified glycopeptide fragment.

78. The method of claim 75, wherein said solid support comprises a hydrazide moiety.

79. The method of claim 75, wherein said glycopeptide fragments are released from said solid support using a glycosidase.

80. The method of claim 79, wherein said glycosidase is an N-glycosidase or an O-glycosidase.

81. The method of claim 79, wherein said glycopeptide fragments are released from said solid support using sequential addition of N-glycosidase and O-glycosidase.

83. The method of claim 76, wherein said glycopeptides are released from said solid support using chemical cleavage.

84. The method of claim 76, wherein said glycopolypeptides are oxidized with periodate.

85. The method of claim 76, wherein said quencher is sodium sulfphite.

86. The method of claim 76, wherein said glycopolypeptides are cleaved with trypsin.

87. The method of claim 76, wherein said sample is selected from a body fluid, secreted proteins, and cell surface proteins.

88. A method of identifying a diagnostic marker for a disease, comprising:
(a) adding a detergent to a test sample and control sample comprising glycopolypeptides;
(b) cleaving glycopolypeptides from said test sample to generate test glycopeptide fragments;
(c) cleaving glycopolypeptides from said control sample to generate control glycopeptide fragments;
(d) adding an oxidizing agent to derivatize said glycopeptide fragments;
(e) adding a quencher to quench said oxidizing agent;
(f) immobilizing said test glycopeptide fragments to a first solid support;
(g) immobilizing said control glycopeptide fragments from a control sample to a second solid support;
(h) releasing the test glycopeptide fragments and control glycopeptide fragments from said solid supports;
(i) analyzing the released glycopeptide fragments; and
(j) identifying one or more glycosylated polypeptides having differential glycosylation between the test sample and the control sample.

89. The method of claim 88, further comprising labeling said immobilized glycopeptide fragments on said first and second supports with differential isotope tags on the respective supports.

90. The method of claim 88, wherein said glycopeptide fragments are oxidized.

91. The method of claim 90, wherein said solid support comprises a hydrazide moiety.

92. The method of claim 90, wherein said glycopeptide fragments are oxidized with periodate.

93. The method of claim 88, wherein said quencher is sodium sulfphite.

94. The method of claim 88, wherein said glycopeptide fragments are released from said solid support using a glycosidase.

95. The method of claim 94, wherein said glycosidase is an N-glycosidase or an O-glycosidase.

96. The method of claim 95, wherein said glycopeptide fragments are released from said solid support using sequential addition of N-glycosidase and O-glycosidase.

97. The method of claim 88, wherein said glycopeptide fragments are released from said solid support using chemical cleavage.

98. The method of claim 88, wherein said glycopolypeptides are cleaved with trypsin.

99. The method of claim 88, wherein the disease is cancer.
